(19) United States
(12) Patent Application Publication
(10) Pub. No.: US 2025/0268979 A1
(43) Pub. Date: Aug. 28, 2025

(54) COMBINATION OF MACROCYCLIC CFTR MODULATORS WITH CFTR CORRECTORS AND / OR CFTR POTENTIATORS

(71) Applicant: Idorsia Pharmaceuticals Ltd, Allschwil (CH)

(72) Inventors: Martin BOLLI, Allschwil (CH); Christine BROTSCHI, Allschwil (CH); John GATFIELD, Allschwil (CH); Hervé SIENDT, Allschwil (CH); Jodi T. WILLIAMS, Allschwil (CH)

(21) Appl. No.: 19/111,782

(22) PCT Filed: Sep. 14, 2023
(86) PCT No.: PCT/EP2023/075269

§ 371 Date: Mar. 13, 2025

(30) Foreign Application Priority Data
Sep. 15, 2022 (WO) PCT/EP2022/075600

(51) Int. Cl. A61K 38/12; A61K 31/352; A61K 31/404; A61K 31/443; A61K 31/4439; A61K 31/47; A61P 11/00

(52) U.S. Cl. CPC A61K 38/12; A61K 31/352; A61K 31/404; A61K 31/443; A61K 31/4439; A61K 31/47; A61P 11/00

(57) ABSTRACT

The present invention concerns the compounds of formula (I) wherein R¹, R², R³, R⁴, X, Ar¹ and Ar² are as described in the description, and their use in the treatment of CFTR-related diseases and disorders, especially in the treatment of cystic fibrosis, in combination with one or more therapeutically active ingredients acting as CFTR modulator(s), wherein said CFTR modulator(s) is/are CFTR one or more CFTR corrector(s) and/or a CFTR potentiator. The invention further relates to pharmaceutical compositions comprising the compounds of formula (I) in combination with one or more therapeutically active ingredients acting as CFTR modulator(s), wherein said CFTR modulator(s) is/are one or more CFTR corrector(s) and/or a CFTR potentiator.

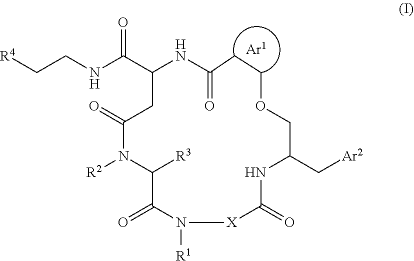

The present invention concerns the compounds of formula (I)

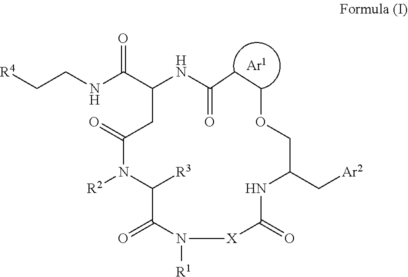

and their use in the treatment of CFTR-related diseases and disorders, in combination with one or more therapeutically active ingredients acting as CFTR modulator(s), wherein said CFTR modulator(s) is/are one or more CFTR corrector(s) (especially a type-I corrector, and/or a type-II corrector, and/or a type-III corrector), and/or a CFTR potentiator; in the prevention and/or treatment of CFTR-related diseases and disorders; wherein such CFTR-related disease is especially cystic fibrosis. The invention further relates to pharmaceutical compositions comprising the compounds of formula (I) in combination with one or more therapeutically active ingredients acting as CFTR modulator(s), wherein said CFTR modulator(s) is/are one or more CFTR corrector(s) (especially a type-I corrector, and/or a type-II corrector, and/or a type-III corrector), and/or a CFTR potentiator.

Certain compounds of formula (I) are disclosed as modulators of CFTR in PCT/EP2021/069292 and may be useful for the treatment of CFTR-related diseases and disorders, especially cystic fibrosis, or of other CFTR-related diseases and disorders selected from:
chronic bronchitis; rhinosinusitis; constipation; pancreatitis; pancreatic insufficiency; male infertility caused by congenital bilateral absence of the vas deferens (CBAVD); mild pulmonary disease; allergic bronchopulmonary aspergillosis (ABPA); liver disease; coagulation-fibrinolysis deficiencies, such as protein C deficiency; and diabetes mellitus;
asthma; COPD; smoke induced COPD; and dry-eye disease; and
idiopathic pancreatitis; hereditary emphysema; hereditary hemochromatosis; lysosomal storage diseases such as especially I-cell disease pseudo-Hurler; mucopolysaccharidoses; Sandhoff/Tay-Sachs;
osteogenesis imperfecta; Fabry disease; Sjogren's disease; osteoporosis; osteopenia; bone healing and bone growth (including bone repair, bone regeneration, reducing bone resorption and increasing bone deposition); chloride channelopathies, such as myotonia congenita (Thomson and Becker forms); Bartter's syndrome type 3; epilepsy; lysosomal storage disease; Primary Ciliary Dyskinesia (PCD)—a term for inherited disorders of the structure and or function of cilia (including PCD with situs inversus also known as Kartagener syndrome, PCD without situs inversus, and ciliary aplasia); generalized epilepsy with fibrile seizures plus (GEFS+); general epilepsy with febrile and afebrile seizures; myotonia; paramyotonia congenital; potassium aggravated myotonia; hyperkalemic periodic paralysis; long QT syndrome (LQTS); LQTS/Brugada syndrome; autosomal-dominant LQTS with deafness; autosomal-recessive LQTS; LQTS with dysmorphic features; congenital and acquired LQTS; dilated cardiomyopathy; autosomal-dominant LQTS; osteopetrosis; and Bartter syndrome type 3.

Cystic Fibrosis (CF; mucoviscidosis, sometimes also called fibrocystic disease of pancreas or pancreatic fibrosis) is an autosomal recessive genetic disease caused by a dysfunctional epithelial chloride/bicarbonate channel named Cystic Fibrosis Transmembrane Conductance Regulator (CFTR). CFTR dysfunction leads to dysregulated chloride, bicarbonate and water transport at the surface of secretory epithelia causing accumulation of sticky mucus in organs including lung, pancreas, liver and intestine and, as a consequence, multi-organ dysfunction. Most debilitating effects in CF are nowadays observed in the lung which-due to abnormal hydration of airway surface liquid, mucus plugging, impaired mucociliary clearance, chronic inflammation and infection-loses its functionality over time leading to death by respiratory failure (Elborn, 2016). Human CFTR is a multidomain protein of 1480 amino acids. Many different mutations causing CFTR dysfunction have been discovered in CF patients leading e.g. to no functional CFTR proteins (class I mutations), CFTR trafficking defects (class II mutations), CFTR regulation defects (also known as gating defects; class III mutations), CFTR conductance defects (class IV mutations), less CFTR protein either due to splicing defects (class V mutations) or due to reduced CFTR stability (class VI mutations), no CFTR protein due to mRNA instability (class VII mutations) (de Boeck, Acta Paediatr. 2020, 109 (5): 893-895). The CFTR2 database (http://cftr2.org; data retrieved 22.08.2022) currently contains information on 401 disease-causing mutations. By far the most common disease-causing mutation is the deletion of phenylalanine at position 508 (F508del; allele frequency 0.697 in the CFTR2 database), that leads to misfolding of the channel during synthesis at the endoplasmic reticulum, degradation of the misfolded protein and a resulting strongly reduced transport to the cell surface (class II mutation). The residual F508del-CFTR that is trafficked to the cell surface is functional, however less than wildtype CFTR, i.e. F508del-CFTR also harbours a gating defect (Dalemans, 1991). Ca 40% of all CF patients are homozygous for the F508del mutation while another ˜40% of patients are heterozygous for the F508del mutation and carry another disease-causing mutation from class I, II, III, IV, V, VI or VII. Such disease-causing mutations are considerably rarer with the class III G551D mutation (allele frequency 0.0210) and the class I G542X mutation (allele frequency 0.0254) and the class II N1303K mutation (allele frequency 0.0158) being the next most prevalent

CF is currently treated by a range of drugs addressing the various organ symptoms and dysfunctions. Intestinal and pancreatic dysfunction are treated from diagnosis by food supplementation with pancreatic digestive enzymes. Lung symptoms are mainly treated with hypertonic saline inhalation, mucolytics, anti-inflammatory drugs, bronchiodilators and antibiotics (Elborn, 2016).

In addition to symptomatic treatments, CFTR modulators have been developed and approved for patients with certain CFTR mutations. These compounds directly improve CFTR folding and trafficking to the cell surface (CFTR correctors) or improve CFTR function at the cell surface (CFTR potentiators). Other types of modulators are still in the exploratory phase such as compounds that increase mRNA levels of (mutated) CFTR (CFTR amplifiers) and compounds that increase the plasma membrane stability of mutated CFTR (CFTR stabilizers), e.g. the nucleotide binding domain 1 (NBD1) stabilizers SION-638, currently in phase 1, and its follow-up molecule NBD1-A (oral presentation G. Hurlbut at the North American Cystic Fibrosis Conference 2022). CFTR modulators can also enhance function of non-mutated (i.e. wildtype) CFTR and are therefore being studied in disorders where increasing wildtype CFTR function would have beneficial effects in non-CF disorders such as chronic bronchitis/COPD/bronchiectasis (Le Grand, J Med Chem. 2021, 64 (11): 7241-7260. Patel, Eur Respir Rev. 2020, 29 (156): 190068) and dry eye disease (Flores, FASEB J. 2016, 30 (5): 1789-1797), and, in addition, acute respiratory distress syndrome (ARDS) (Erfinanda L, Sci Transl Med. 2022, 14 (674): eabg8577).

CFTR modulators and their combinations can be discovered and optimized by assessing their ability to promote trafficking and function of mutated CFTR in in vitro cultivated recombinant and primary cellular systems. Activity in such systems is predictive of activity in CF patients.

WO2019/161078 discloses macrocycles as modulators of cystic fibrosis, wherein said macrocycles generally are 15-membered macrocycles comprising a (pyridine-carbonyl)-sulfamoyl moiety that is linked to a further aromatic group. Further macrocycles are disclosed in WO2022/109573 (macrocycles containing a 1,3,4-oxadiazole ring), WO2022/076625, WO2022/076626, WO2022/076624, WO2022/076621, WO2022/076620, WO2022/076618, WO2021/030556, and WO2021/030555. Macrocyclic tetrapeptides (12- or 13-membered) including the compound Apicidin (CAS: 183506-66-3) have been proposed as potential agents for treating CF (Hutt D M et al. ACS Med Chem Lett. 2011; 2 (9): 703-707. doi: 10.1021/ml200136e). WO2020/128925 discloses macrocycles capable of modulating the activity of CFTR, wherein said macrocycles comprise an optionally substituted divalent N-(pyridine-2-yl)pyridinyl-sulfonamide moiety. Other macrocyclic compounds have been described to stabilize chloride channel CFTR (Stevers L. M., Nature Communications 2022, 13:3586). Non macrocyclic CFTR correctors and/or potentiators of CFTR have been disclosed for example in WO2011/119984, WO2014/015841, WO2007/134279, WO2010/019239, WO2011/019413, WO2012/027731, WO2013/130669, WO2014/078842 and WO2018/227049, WO2010/037066, WO2011/127241, WO2013/112804, WO2014/071122, and WO2020/128768. Furthermore, particular macrocycles can be found as screening compounds, wherein an unsubstituted phenylene group is part of said macrocycles compared to the 8- to 10-membered bicyclic heteroarylene of present compounds of formula (I) (CAS registry number: CAS-2213100-89-9, CAS-2213100-96-8, CAS-2213100-99-1, CAS-2213101-02-9, CAS-2213101-04-1, CAS-2213101-06-3, CAS-2213101-08-5, CAS-2213101-09-6, CAS-2213101-19-8, CAS-2213101-24-5, CAS-2215788-95-5, CAS-2215788-98-8, CAS-2215789-01-6, CAS-2215789-02-7, CAS-2215789-09-4, CAS-2215789-15-2, CAS-2215789-20-9, CAS-2215789-24-3, CAS-2215789-35-6, CAS-2215789-37-8, CAS-2215946-94-2, CAS-2215947-04-7, CAS-2215947-13-8, CAS-2215947-24-1, CAS-2215947-34-3, CAS-2215947-44-5, CAS-2215947-51-4, CAS-2215947-64-9, CAS-2215947-68-3, CAS-2215947-78-5, CAS-2215947-91-2, CAS-2215954-57-5, CAS-2216342-34-4, CAS-2216342-78-6, CAS-2216342-86-6, CAS-2216343-03-0, CAS-2216343-09-6, CAS-2216343-14-3, CAS-2216343-18-7, CAS-2216343-24-5, CAS-2216343-32-5, CAS-2216343-38-1, CAS-2216343-45-0, CAS-2216343-53-0, CAS-2216343-59-6, CAS-2216343-64-3, CAS-2216343-74-5, CAS-2216343-76-7).

CFTR modulators can be further subdivided into-among others-CFTR correctors and CFTR potentiators. CFTR correctors improve folding of CFTR and its trafficking to the cell surface, especially of CFTR carrying class II (=folding & trafficking) mutations, and thus increase CFTR cell surface expression. CFTR potentiators increase the opening probability of cell surface CFTR, especially of CFTR carrying gating defects including corrector-rescued class II mutants, and therefore can activate CFTR in an additive/synergistic manner together with CFTR correctors. Thus, potentiators and correctors have been/are being combined in the clinic to treat patients with CFTR class II mutations. A multitude of CFTR correctors have been described in the literature and in patents. Some of these correctors—alone and/or in combination with other CFTR modulators—have been/are being used in clinical trials in CF patients, e.g. VX-809 (lumacaftor), VX-661 (tezacaftor), ABBV-2222 (galicaftor), VX-445 (elexacaftor), VX-659 (bamocaftor), VX-440 (olacaftor), VX-121 (vanzacaftor), ABBV-C2 correctors-ABBV-119, and ABBV-567, and, in addition to the before-listed, PTI-801. Similarly, a multitude of potentiators has been described in the literature and in patents. Some of these potentiators—alone and/or in combination with other CFTR modulators—have been/are being used in clinical trials in CF patients, e.g. VX-770 (ivacaftor), VX-561 (deutivacaftor), GLPG-1837, GLPG-2451, ABBV-3067 (navocaftor), QBW-251 (icenticaftor).

It has been established that CFTR correctors can be further subdivided with respect to their mechanism: Correctors behaving in an additive or synergistic way must have different, i.e. complementary, mechanims of action, most likely due to different binding sites on the CFTR protein. Inversely, correctors behaving in a competitive way likely share the same CFTR binding site (Okiyoneda, 2013; Veit, 2018; Veit, 2020; Fiedorczuk 2022; Marchesin 2023). Accordingly, the structurally related correctors VX-809 (lumacaftor), VX-661 (tezacaftor) and ABBV-2222 (galicaftor) have been categorized as type-I correctors, Corrector 4a and related compounds have been categorized as type-II correctors while VX-445 (elexacaftor) has been described as type-III corrector, which applies also to the structurally related correctors VX-440 (olacaftor), VX-659 (bamocaftor) and VX-121 (vanzacaftor). ABBV-C2 correctors ABBV-119, ABBV-567 are likely type-III correctors as well. PTI-801 is likely a further type-III corrector. Due to their additivity in effect, correctors of different mechanisms are being combined in the clinic to reach-together with a potentiator-higher correction efficacy.

VX-770 (ivacaftor, KALYDECO, N-(2,4-di-tert-butyl-5-hydroxyphenyl)-1,4-dihydro-4-oxoquinoline-3-carboxamide, CAS 873054-44-5, e.g. WO2006/002421, WO2011/072241, WO2007/079139, WO2007/134279, WO2010/019239, WO2013/130669), is a CFTR potentiator and the first ever approved CFTR modulator (US and EU: initial approval 2012) for treatment of CF patients aged 4 months and older who have one mutation in the CFTR gene that is responsive to KALYDEKO based on clinical and/or in vitro assay data. The current VX-770 uspi (December 2020) lists 97 elegible CFTR mutations. VX-770 is also part of ORKAMBI (combination of VX-809 and VX-770), SYMDEKO/SYMKEVI (combination of VX-661 and VX-770) and TRIKAFTA/KAFTRIO (combination of VX-661, VX-445 and VX-770) as described later.

The efficacy of KALYDECO in patients with CF who have a G551D mutation in the CFTR gene was evaluated in two phase-III clinical trials in 213 patients with CF. Trial 1 (NCT00909532) evaluated patients who were at least 12 years of age, trial 2 (NCT00909727) evaluated patients who were 6 to 11 years of age. Patients were randomized 1:1 to receive either KALYDECO or placebo with food containing fat for 48 weeks in addition to their prescribed CF therapies (e.g., tobramycin, dornase alfa). The primary efficacy endpoint was improvement in lung function as determined by the mean absolute change from baseline in percent predicted pre-dose FEV1 through 24 weeks of treatment. FEV1 stands for “forced expiratory volume in 1 second” and is the commonly used primary endpoint capturing lung function in CF clinical trials. In both studies, treatment with KALYDECO resulted in a significant absolute change of percent predicted FEV1 (ppFEV1) of at least 10% compared to placebo. Some secondary endpoints were favorably changed as well on KALYDECO after 48 weeks of treatment such as risk of pulmonary exacerbations (trial 1: relative risk reduction of 0.4 vs placebo), body weight (ca +2 kg vs placebo) and the pharmacodynamic biomarker sweat chloride (ca-50 mM vs placebo). In other smaller phase-III trials, KALYDECO was tested in CF patients of at least 6 years of age with rarer mutations predicted to respond using change from baseline in absolute ppFEV1 as primary end point. Furthermore, KALYDECO has been assessed in CF patients with ages between 4 months and 5 years without assessing efficacy via FEV1 measurements, however reaching KALYDECO exposures and sweat chloride reduction similar to those seen in the trials using older patients. The recommended dosage of KALYDECO for adults and pediatric patients ages 6 years and older is one 150 mg tablet taken orally every 12 hours (b.i.d. dosing). The recommended dosage of KALYDECO (oral granules) for patients ages 4 months to less than 6 years is weight-based with patients between 4-6 months taking 25 mg b.i.d. (if weighing >5 kg), patients between 6 months and below 6 years taking 25 mg b.i.d. (if weighing 5-7 kg), 50 mg b.i.d. (if weighing 7-14 kg) and 75 mg b.i.d. (if weighing 14-25 kg). The FDA recommends to reduce the dosage of KALYDECO to one tablet or one packet of oral granules once daily for patients aged 6 months and older with moderate hepatic impairment (Child-Pugh Class B). KALYDECO should be used with caution in patients aged 6 months and older with severe hepatic impairment (Child-Pugh Class C), at a dosage of one tablet or one packet of oral granules once daily or less frequently. Use is not recommended in patients with hepatic impairment below 6 months of age. KALYDECO is available as a light blue, capsule shaped, film-coated tablet for oral administration containing 150 mg of ivacaftor. Each KALYDECO tablet contains the following inactive ingredients: colloidal silicon dioxide, croscarmelose sodium, hypromellose acetate succinate, lactose monohydrate, magnesium stearate, microcrystalline cellulose, and sodium lauryl sulfate. The tablet film coat contains carnauba wax, FD&C Blue #2, PEG 3350, polyvinyl alcohol, talc, and titanium dioxide. The printing ink contains ammonium hydroxide, iron oxide black, propylene glycol, and shellac.

KALYDECO is also available as white to off-white granules for oral administration (sweetened but unflavored) and enclosed in a unit-dose packet containing 25 mg, 50 mg or 75 mg of ivacaftor. Each unit-dose packet of KALYDECO oral granules contains in addition the following inactive ingredients: colloidal silicon dioxide, croscarmellose sodium, hypromellose acetate succinate, lactose monohydrate, magnesium stearate, mannitol, sucralose, and sodium lauryl sulfate.

The overall safety profile of KALYDECO is based on pooled data from three placebo-controlled clinical trials conducted in 353 patients 6 years of age and older with CF who had a G551D mutation in the CFTR gene or were homozygous for the F508del mutation (NCT00909532, NCT00909727, NCT00953706). 221 patients received KALYDECO, and 132 received placebo from 16 to 48 weeks. Serious adverse reactions, whether considered drug-related or not by the investigators, that occurred more frequently in KALYDECO-treated patients included abdominal pain, increased hepatic enzymes, and hypoglycemia. Elevated transaminases have been reported in patients with CF receiving KALYDECO. The FDA recommends to assess ALT and AST prior to initiating KALYDECO, every 3 months during the first year of treatment, and annually thereafter. Dosing should be interrupted in patients with ALT or AST of greater than 5 times the upper limit of normal (ULN). Following resolution of transaminase elevations, the FDA recommends to consider the benefits and risks of resuming KALYDECO. Non-congenital lens opacities/cataracts have been reported in pediatric patients treated with KALYDECO. The FDA recommends baseline and follow-up examinations in pediatric patients initiating KALYDECO treatment.

VX-809 (lumacaftor, 3-[6-({[1-(2,2-difluoro-1,3-benzodioxol-5-yl)cyclopropyl]carbonyl}amino)-3-methylpyridin-2-yl]benzoic acid, CAS 936727 May 8, e.g. WO2007/056341, WO2009/073757, WO2009/076141, WO2010/037066, WO2011/127241) is a type-I CFTR corrector, and was approved in the United States (2014) and the EU (2015) as part of ORKAMBI, which is a combination product of lumacaftor with the CFTR potentiator ivacaftor for the treatment of cystic fibrosis (CF) in patients age 2 years and older who are homozygous for the F508del mutation in the CFTR gene.

The efficacy of ORKAMBI in patients with CF homozygous for the F508del mutation was evaluated in two phase III clinical trials (NCT01807923, NCT01807949) with a total of 1108 patients who were at least 12 years of age of whom 369 patients received ORKAMBI (lumacaftor 400 mg/ivacaftor 250 mg, b.i.d.) for 24 weeks in addition to their prescribed CF therapies (e.g., bronchodilators, inhaled antibiotics, dornase alfa, and hypertonic saline). The primary efficacy endpoint was change in lung function as determined by absolute change from baseline in ppFEV1 at Week 24, and ORKAMBI treatment resulted in a statistically significant increase of 2.6% and 3.0% in absolute ppFEV1 compared to placebo treatment. The secondary endpoint risk of pulmonary exacerbations was favorably changed as well on ORKAMBI (relative risk reduction of 0.3-0.4 vs placebo). In small open label phase III trials, ORKAMBI was tested in CF patients aged 6 through 11 homozygous for the F508del mutation measuring sweat chloride concentrations as marker of CFTR function. The mean absolute change from baseline in sweat chloride was −20.4 mM at Day 15 and −24.8 mM at Week 24. After a 2-week washout period the mean absolute sweat chloride concentrations increased by 21.3 mM. Similarly, in F508del-CFTR homozygous CF patients aged 2 through 5, the mean absolute change from baseline in sweat chloride was-31.7 mM at week 24 followed by an increase of 33 mM after a 2-week washout period. These data indicate improvement of CFTR function. The efficacy of ORKAMBI in children ages 2 through 11 years was extrapolated from efficacy in patients ages 12 years and older homozygous for the F508del mutation with support from population pharmacokinetic analyses showing similar drug exposure levels in patients ages 12 years and older and in children ages 2 through 11 years. The recommended dosage of ORKAMBI for adults and pediatric patients ages 12 years and older is two tablets (each containing lumacaftor 200 mg/ivacaftor 125 mg) taken orally every 12 hours for a daily total of lumacaftor 800 mg/ivacaftor 500 mg. The recommended dosage of ORKAMBI for patients age 6 through 11 is two tablets (each containing lumacaftor 100 mg/ivacaftor 125 mg) taken orally b.i.d. for a daily total of lumacaftor 400 mg/ivacaftor 500 mg. The dosage of ORKAMBI for patients age 2 through 5 years is weight dependent. Above 14 kg one packet of granules (containing lumacaftor 150 mg/ivacaftor 188 mg) taken orally b.i.d. is recommended. Below 14 kg one packet of granules (containing lumacaftor 100 mg/ivacaftor 125 mg) taken orally b.i.d. is recommended. The FDA recommends to reduce the dosage of ORKAMBI to two tablets in the morning and 1 tablet in the evening for patients aged 6 years and older with moderate hepatic impairment (Child-Pugh Class B) and to 1 package of granules daily in patients aged 2 through 5 with moderate hepatic impairment. In patients with severe hepatic impairment (Child-Pugh Class C), ORKAMBI needs to be used with caution at a maximum dose of 1 tablet in the morning and 1 tablet in the evening or less frequently, or 1 packet of oral granules once daily or less frequently. ORKAMBI is available as pink, oval-shaped, film-coated, fixed-dose combination tablets containing 100 mg of lumacaftor and 125 mg of ivacaftor, or 200 mg lumacaftor and 125 mg ivacaftor. In addition, the following inactive ingredients are present: cellulose, microcrystalline; croscarmellose sodium; hypromellose acetate succinate; magnesium stearate; povidone; and sodium lauryl sulfate. The tablet film coat contains carmine, FD&C Blue #1, FD&C Blue #2, polyethylene glycol, polyvinyl alcohol, talc, and titanium dioxide. The printing ink contains ammonium hydroxide, iron oxide black, propylene glycol, and shellac. ORKAMBI is also available as oral granules with unit-dose packets containing lumacaftor 100 mg/ivacaftor 125 mg or lumacaftor 150 mg/ivacaftor 188 mg per packet. In addition, the following inactive ingredients are present: cellulose, microcrystalline; croscarmellose sodium; hypromellose acetate succinate; povidone; sodium lauryl sulfate. The overall safety profile of ORKAMBI is based on the pooled data from 1108 patients with CF 12 years and older homozygous for the F508del mutation and who received at least one dose of study drug in 2 double-blind, placebo-controlled, phase III clinical trials, each with 24 weeks of treatment. 369 patients received ORKAMBI b.i.d. and 370 received placebo. Serious adverse reactions, whether considered drug-related or not by the investigators, that occurred more frequently in patients treated with ORKAMBI included pneumonia, hemoptysis, cough, increased blood creatine phosphokinase, and transaminase elevations. These occurred in 1% or less of patients. Elevated transaminases (ALT/AST) have been observed in some cases associated with elevated bilirubin. The FDA recommends to measure serum transaminases and bilirubin before initiating ORKAMBI, every 3 months during the first year of treatment, and annually thereafter. Dosing should be interrupted in patients with ALT or AST >5× upper limit of normal (ULN), or ALT or AST >3×ULN with bilirubin >2×ULN. Following resolution, the FDA recommends to consider the benefits and risks of resuming ORKAMBI. Furthermore, respiratory side effects (chest discomfort, dyspnea, and respiration abnormal) were observed more commonly during initiation of ORKAMBI and in patients with ppFEV1<40% and additional monitoring is recommended during initiation of therapy. Also, increased blood pressure has been observed in some patients and the FDA recommends periodical monitoring of blood pressure in all patients. Non-congenital lens opacities/cataracts have been reported in pediatric patients treated with ORKAMBI. The FDA recommends baseline and follow-up examinations in pediatric patients initiating ORKAMBI treatment.

VX-661 (tezacaftor, 1-(2,2-difluoro-2H-1,3-benzodioxol-5-yl)-N-{1-[(2R)-2,3-dihydroxypropyl]-6-fluoro-2-(1-hydroxy-2-methylpropan-2-yl)-1Hindol-5-yl}cyclopropane-1-carboxamide, CAS 1152311-62-0, e.g. WO2007/117715, WO2011/119984, WO2014/014841) is a type-I CFTR corrector, and was approved in the United States (2018) as part of SYMDEKO and in the EU (2018) as part of SYMKEVI. SYMDEKO/SYMKEVI is a combination product of tezacaftor with the CFTR potentiator ivacaftor for the treatment of cystic fibrosis (CF) in patients age 6 years and older who are homozygous for the F508del mutation in the CFTR gene or who have at least one mutation in the CFTR gene that is responsive to tezacaftor/ivacaftor based on in vitro data and/or clinical evidence. The current SYMDEKO uspi (June 2022) lists 154 elegible CFTR mutations.

The efficacy of SYMDEKO in patients with CF age 12 years and older was evaluated in three double-blind, placebo-controlled trials (Trials 1, 2, and 3). Patients in all trials continued on their standard-of-care CF therapies (e.g., bronchodilators, inhaled antibiotics, dornase alfa, and hypertonic saline). Trial 1 (NCT02347657) evaluated 504 patients homozygous for the F508del-CFTR mutation. Patients were randomized 1:1 to receive either SYMDEKO or placebo with food containing fat. The primary efficacy endpoint was improvement in lung function as determined by the mean absolute change from baseline in ppFEV1 through 24 weeks of treatment. Treatment with SYMDEKO resulted in a significant absolute change of ppFEV1 of 4% compared to placebo. Some secondary endpoints were favorably changed as well on SYMDEKO after 24 weeks of treatment such as risk of pulmonary exacerbations (relative risk reduction of 0.35 vs placebo), absolute change in CFQ-R Respiratory Domain Score (a measure of respiratory symptoms relevant to patients with CF, such as cough, sputum production, and difficulty breathing) from baseline through week 24: +5.1) and absolute change from baseline in sweat chloride through week 24 (−10.1 mM compared to placebo).

Trial 2 (NCT02392234) in a cross-over design evaluated 244 patients heterozygous for the F508del mutation and a second mutation predicted to be responsive to tezacaftor/ivacaftor (146 patients had a splice mutation and 98 patients had a missense mutation as the second allele). The primary efficacy endpoint was improvement in lung function as determined by the mean absolute change from baseline in ppFEV1 averaged at week 4 and week 8 of treatment. Treatment with SYMDEKO resulted in a significant absolute change of ppFEV1 of 6.8% compared to placebo. Some secondary endpoints were favorably changed as well on SYMDEKO such as absolute change in CFQ-R Respiratory Domain Score from baseline (+11.1 versus placebo) and the CFTR functional biomarker sweat chloride (−9.5 mM). Trial 3 (NCT02516410) evaluated SYMDEKO in 168 patients who were heterozygous for the F508del mutation and a second mutation not predicted to be responsive to tezacaftor/ivacaftor. No significant changes in absolute ppFEV1 from baseline through week 12 were observed in the SYMDEKO group when compared to the placebo group.

The effectiveness of SYMDEKO in patients age 6 to less than 12 years was extrapolated from patients age 12 years and older with support from population pharmacokinetic analyses showing similar tezacaftor and ivacaftor exposure levels in patients age 6 to less than 12 years (trial 4; NCT02953314) open-label study in 70 patients) and in patients age 12 years and older (trials 1 and 2). Also, in trial 4, a change in sweat chloride was observed from baseline through week 24 of −14.5 mM indicating improved CFTR function.

The recommended dosage of SYMDEKO for patients of 12 years and older and pediatric patients age 6 years and older if weighing at least 30 kg is one tablet (containing tezacaftor 100 mg/ivacaftor 150 mg) in the morning and one tablet (containing ivacaftor 150 mg) in the evening for a daily total of tezacaftor 100 mg/ivacaftor 300 mg. The recommended dosage of SYMDEKO for patients age 6 through 11 if weighing less than 30 kg is one tablet (containing tezacaftor 50 mg/ivacaftor 75 mg) in the morning and one tablet (containing ivacaftor 75 mg) in the evening for a daily total of tezacaftor 50 mg/ivacaftor 150 mg. The FDA recommends to reduce the dosage of SYMDEKO to the morning tablet omitting the evening ivacaftor dose in patients with moderate hepatic impairment (Child-Pugh Class B). In patients with severe hepatic impairment (Child-Pugh Class C), SYMDEKO needs to be used with caution at a maximum dose of 1 morning tablet.

SYMDEKO is available as white, capsule-shaped, fixed-dose tezacaftor/ivacaftor combination tablets co-packaged with light-blue capsule-shaped ivacaftor tablets. Packages of tezacaftor 50 mg/ivacaftor 75 mg+ivacaftor 75 mg and of tezacaftor 100 mg/ivacaftor 150 mg+ivacaftor 150 mg are available. In the tezacaftor/ivacaftor combination tablets the following inactive ingredients are present: croscarmellose sodium, hypromellose, hypromellose acetate succinate, magnesium stearate, microcrystalline cellulose and sodium lauryl sulfate. The tablet film coat contains HPMC/hypromellose 2910, hydroxypropyl cellulose, talc and titanium dioxide. The ivacaftor tablet contains the following inactive ingredients: colloidal silicon dioxide, croscarmellose sodium, hypromellose acetate succinate, lactose monohydrate, magnesium stearate, microcrystalline cellulose, and sodium lauryl sulfate. The tablet film coat contains carnauba wax, FD&C Blue #2, PEG 3350, polyvinyl alcohol, talc, and titanium dioxide. The printing ink contains ammonium hydroxide, iron oxide black, propylene glycol, and shellac.

The overall safety profile of SYMDEKO is based on data from 1001 patients with CF in three double-blind, placebo-controlled, clinical trials: two parallel-group trials of 12 and 24 week duration (NCT02347657, NCT02516410) and one cross-over design trial of 8 weeks duration (NCT02392234). Eligible patients were also able to participate in an open-label extension safety study (up to 96 weeks of SYMDEKO). In the three placebo-controlled trials a total of 496 patients with CF age 12 years and older received at least one dose of SYMDEKO. Serious adverse reactions, whether considered drug-related or not by the investigators, that occurred more frequently in SYMDEKO-treated patients compared to placebo included distal intestinal obstruction syndrome, 3 (0.6%) SYMDEKO-treated patients vs. 0 placebo. Elevated transaminases (ALT/AST) have been observed in patients treated with SYMDEKO. The FDA recommends to measure serum transaminases before initiating SYMDEKO, every 3 months during the first year of treatment, and annually thereafter. Dosing should be interrupted in patients with ALT or AST >5× upper limit of normal (ULN), or ALT or AST >3×ULN with bilirubin >2×ULN. Following resolution, the FDA recommends to consider the benefits and risks of resuming SYMDEKO. Non-congenital lens opacities/cataracts have been reported in pediatric patients treated with SYMDEKO. The FDA recommends baseline and follow-up examinations in pediatric patients initiating SYMDEKO treatment.

VX-445 (elexacaftor, N-(1,3-dimethyl-1H-pyrazole-4-sulfonyl)-6-[3-(3,3,3-trifluoro-2,2-dimethylpropoxy)-1H-pyrazol-1-yl]-2-[(4S)-2,2,4-trimethylpyrrolidin-1-yl]pyridine-3-carboxamide, CAS 2216712-66-0, e.g. WO2016/057572, WO2018/107100, WO2019/152940) is a type-III CFTR corrector, and was approved in the United States (2019) as part of TRIKAFTA and in the EU (2020) as part of KAFTRIO. TRIKAFTA/KAFTRIO is a combination product of elexacaftor with the type-I CFTR corrector tezacaftor and the CFTR potentiator ivacaftor for the treatment of cystic fibrosis (CF) in patients age 6 years and older who have at least one F508del mutation in the CFTR gene or a mutation in the CFTR gene that is responsive based on in vitro data. The current TRIKAFTA uspi (October 2021) lists 178 elegible CFTR mutations.

The efficacy of TRIKAFTA in patients with CF age 12 years and older was evaluated in two double-blind, placebo-controlled trials (Trials 1 and 2). Patients discontinued any previous CFTR modulator therapies, but continued on their other standard-of-care CF therapies (e.g., bronchodilators, inhaled antibiotics, dornase alfa and hypertonic saline). Trial 1 (NCT03525444) evaluated 403 patients (200 TRIKAFTA, 203 placebo) that had an F508del mutation on one allele and a mutation on the second allele that results in either no CFTR protein or a CFTR protein that is not responsive to ivacaftor and tezacaftor/ivacaftor. The primary efficacy endpoint was improvement in lung function as determined by the mean absolute change from baseline in ppFEV1 through 4 weeks of treatment at the time point of interim analysis. The treatment difference between TRIKAFTA and placebo was 13.8%. Key secondary endpoints determined at 24 weeks of treatment were favorably changed as well on TRIKAFTA such as risk of pulmonary exacerbations (relative risk reduction of 0.63 vs placebo), absolute change in CFQ-R Respiratory Domain Score from baseline (+20.2 versus placebo), absolute change in body mass index (BMI) from baseline (+1.04 kg/m² versus placebo) and absolute change from baseline in sweat chloride (−41.8 mM versus placebo).

Trial 2 (NCT03525548) evaluated 107 patients with CF aged 12 years and older homozygous for the F508del mutation. Following the 4-week open-label run-in period with tezacaftor/ivacaftor patients were randomized to TRIKAFTA or continued on tezacaftor/ivacaftor for 4 weeks. The primary endpoint was mean absolute change in ppFEV1 from baseline at Week 4 of the double-blind treatment period, and treatment with TRIKAFTA compared to tezacaftor/ivacaftor resulted in a statistically significant improvement in ppFEV1 of 10.0% Key secondary endpoints at 4 weeks were favorably changed as well on TRIKAFTA versus tezacaftor/ivacaftor such as absolute change in CFQ-R Respiratory Domain Score from baseline (+17.4) and the CFTR functional biomarker sweat chloride (−45.1 mM).

Trial 3 (NCT03691779) was an open-label trial evaluating TRIKAFTA in 66 children aged 6 to less than 12 years who were homozygous for the F508del mutation or heterozygous for the F508del mutation with a mutation on the second allele that results in either no CFTR protein or a CFTR protein that is not responsive to ivacaftor and tezacaftor/ivacaftor. The mean absolute change in sweat chloride from baseline through Week 24 was-60.9 mmol/L. The effectiveness of TRIKAFTA in patients aged 6 to less than 12 years was extrapolated from patients aged 12 years and older with support from population pharmacokinetic analyses showing elexacaftor, tezacaftor and ivacaftor exposure levels in patients aged 6 to less than 12 years within the range of exposures observed in patients aged 12 years and older (Trials 1 and 2).

The recommended dosage of TRIKAFTA for patients of 12 years and older and pediatric patients aged 6 years and older if weighing at least 30 kg is two tablets (each containing elexacaftor 100 mg/tezacaftor 50 mg/ivacaftor 75 mg) in the morning and one tablet of ivacaftor 150 mg in the evening for a daily total of elexacaftor 200 mg/tezacaftor 100 mg/ivacaftor 300 mg. The recommended dosage of TRIKAFTA for patients aged 6 through 11 if weighing less than 30 kg is two tablets (each containing elexacaftor 50 mg/tezacaftor 25 mg/ivacaftor 37.5 mg) in the morning and one tablet (containing ivacaftor 75 mg) in the evening for a daily total of elexacaftor 100 mg/tezacaftor 50 mg/ivacaftor 150 mg. The FDA does not recommend the use of TRIKAFTA in patients with moderate hepatic impairment (Child-Pugh Class B). However if used, TRIKAFTA dose should be reduced by applying the morning tablets only in an alternating schedule of 1 and 2 morning tablets daily.

TRIKAFTA is a co-package of elexacaftor, tezacaftor and ivacaftor fixed-dose combination tablets and ivacaftor tablets. Both tablets are for oral administration. The elexacaftor, tezacaftor and ivacaftor fixed-dose combination tablets are orange, capsule-shaped and film-coated containing 100 mg of elexacaftor, 50 mg of tezacaftor, 75 mg of ivacaftor, or light-orange, capsule-shaped and film-coated containing 50 mg of elexacaftor, 25 mg of tezacaftor, 37.5 mg of ivacaftor. The following inactive ingredients are present: hypromellose, hypromellose acetate succinate, sodium lauryl sulfate, croscarmellose sodium, microcrystalline cellulose and magnesium stearate. The tablet film coat contains hypromellose, hydroxypropyl cellulose, titanium dioxide, talc, iron oxide yellow and iron oxide red. The ivacaftor tablet is available as a light blue, capsule-shaped, film-coated tablet containing 150 mg or 75 mg of ivacaftor and the following inactive ingredients: colloidal silicon dioxide, croscarmellose sodium, hypromellose acetate succinate, lactose monohydrate, magnesium stearate, microcrystalline cellulose and sodium lauryl sulfate. The tablet film coat contains carnauba wax, FD&C Blue #2, PEG 3350, polyvinyl alcohol, talc and titanium dioxide. The printing ink contains ammonium hydroxide, iron oxide black, propylene glycol and shellac.

The safety profile of TRIKAFTA is based on data from 510 CF patients aged 12 years and older in two double-blind, controlled trials of 24 weeks and 4 weeks treatment duration (Trials 1 and 2). Eligible patients were also able to participate in an open-label extension safety study (up to 96 weeks of TRIKAFTA). In the two controlled trials, a total of 257 patients aged 12 years and older received at least one dose of TRIKAFTA. In Trial 1, serious adverse reactions that occurred more frequently in TRIKAFTA-treated patients compared to placebo were rash (1% vs<1%) and influenza (1% vs 0). The most common adverse drug reactions to TRIKAFTA (>5% of patients and at a frequency higher than placebo by ≥1%) were headache, upper respiratory tract infection, abdominal pain, diarrhea, rash, alanine aminotransferase increased, nasal congestion, blood creatine phosphokinase increased, aspartate aminotransferase increased, rhinorrhea, rhinitis, influenza, sinusitis and blood bilirubin increased.

In trial 1, the incidence of adverse reactions of transaminase elevations (AST and/or ALT) was 11% in TRIKAFTA-treated patients and 4% in placebo-treated patients. The incidence of maximum total bilirubin elevation >2×ULN was 4% in TRIKAFTA-treated patients and <1% in placebo-treated patients. Liver failure leading to transplantation has been reported in a patient with cirrhosis and portal hypertension while receiving TRIKAFTA. The FDA recommends to avoid use of TRIKAFTA in patients with pre-existing advanced liver disease unless the benefits are expected to outweigh the risks. If used in these patients, they should be closely monitored after the initiation of treatment. The FDA recommends assessments of liver function tests (ALT, AST, and bilirubin) for all patients prior to initiating TRIKAFTA, every 3 months during the first year of treatment, and annually thereafter. In the event of significant elevations in liver function tests, e.g., ALT or AST >5× the upper limit of normal (ULN) or ALT or AST >3×ULN with bilirubin >2×ULN, dosing should be interrupted, and laboratory tests closely followed until the abnormalities resolve. Following resolution, the FDA recommends to consider the benefits and risks of resuming TRIKAFTA.

Non-congenital lens opacities/cataracts have been reported in pediatric patients treated with ivacaftor-containing regimens. The FDA recommends baseline and follow-up examinations in pediatric patients initiating TRIKAFTA treatment. Further potential side effects of TRIKAFTA are reported to include rash (Trial 1 10% on TRIKAFTA vs 5% on placebo) and elevated blood pressure. Maximum increase from baseline in mean systolic and diastolic blood pressure was 3.5 mmHg and 1.9 mmHg, respectively for TRIKAFTA-treated patients (baseline: 113 mmHg systolic and 69 mmHg diastolic) and 0.9 mmHg and 0.5 mmHg, respectively for placebo-treated patients (baseline: 114 mmHg systolic and 70 mmHg diastolic).

ABBV-2222 (GLPG-2222, galicaftor, 4-[(2R,4R)-4-({[1-(2,2-Difluoro-1,3-benzodioxol-5-yl)cyclopropyl]carbonyl}amino)-7-(difluoromethoxy)-3,4-dihydro-2H-chromen-2-yl]benzoic Acid; CAS 1918143-53-9, e.g. WO2016/069757) is a type-I CFTR corrector, that is currently being tested in combination with the CFTR potentiator ABBV-3067 (see later) in an open-label phase-II trial (NCT03969888) in 78 CF patient aged 18 years and older homozygous for the F508del-CFTR mutation. Primary endpoint is absolute change in ppFEV1 at day 29 from baseline, secondary endpoints include absolute change in sweat chloride at day 29 from baseline and change in forced vital capacity (the total amount of air exhaled during forced expiratory volume test, a lung function test). Several not disclosed dosing regimens of galicaftor and navocaftor are being tested. In addition, galicaftor was tested as part of a triple combination of CFTR modulators in an open label phase-II trial (NCT04853368) in which one of the cohorts of homozygous F508del-CFTR patients was treated for 28 d with a fixed dose of galicaftor and potentiator navocaftor followed by a 28 day treatment with the triple combination of galicaftor/navocaftor and the CFTR corrector ABBV-119 that has a mechanism different from galicaftor. Interim analysis after the dual treatment phase indicated efficacy of the galicaftor/navocaftor dual combination on ppFEV1 (“performed well”) while the addition of the ABBV-119 did not add further efficacy leading to discontinuation of this clinical trial (Abbvie oral communication at press conference Apr. 29, 2022). Previously, galicaftor has been tested as single agent in a placebo-controlled phase-IIa study (NCT03119649) in which 59 CF patients aged 18 year and older homozygous for the F508del-CFTR mutation were treated for 4 weeks at once-daily doses of 50, 100, 200 and 400 mg. Primary endpoint was number of participants with treatment-emergent adverse events through day 43, key secondary end points were change from baseline in ppFEV1 at day 29, change from baseline in CFQ-R at day 29 and change from baseline in sweat chloride concentration at day 29. Galicaftor was well tolerated and safe. No significant changes versus placebo were observed for ppFEV1 and in the CFQ-R score. Change in sweat chloride was significant in the 200 mg dose group (−15.8 mM versus placebo). The data indicate that galicaftor in single treatment mode at the tested doses has no effect on lung function in homozygous F508del-CFTR CF patients. In vitro, using differentiated human bronchial epithelial cells from homozygous F508del-CFTR doners, galicaftor displayed high potency (EC₅₀=5 nM) in electrophysiological recordings (trans-epithelial current clamp) in the presence of a potentiator GLPG1837 (Wang, 2018). Furthermore, galicaftor has been tested in a phase-Ib open label trial (NCT03540524) in combination with another potentiator, GLPG-2451, in a fixed dose combination. Ten patients with CF homozygous for F508del-CFTR mutation were treated for 2 weeks with the galicaftor/GLPG-2451 combination before the addition of another CFTR modulator (GLPG-2737). Interim analysis at 2 weeks of treatment with galicaftor and GLPG-2451 showed that the combination was well tolerated and safe during the 2 week treatment phase. Furthermore, key secondary endpoints had improved versus baseline: ppFEV1 (+3%) and sweat chloride (−25 mM) (Press Release Galapagos Oct. 24, 2018) indicating that galicaftor can partially correct F508del-CFTR function when combined with a potentiator.

PTI-801 (posenacaftor, CAS 2095064 May 2; compound of example 2 of WO 2019/071078) is classifiable, per the Examples of the present application, as a type-III CFTR corrector. PTI-801 was most recently tested in combination with the CFTR potentiator PTI-808 and CFTR amplifier PTI-428 in a phase 1/2, randomized, double-blind, placebo-controlled study (NCT03500263). 31 CF patients aged 18 years and older carrying at least one F508del-CFTR allele were randomized to receive either the triple treatment combination or a placebo. The triple combo was given daily with a fixed dose of PTI-428 (30 mg) plus either a low dose of PTI-801 (200 mg) and a high-dose-tested of PTI-808 (300 mg), or a high-dose-tested of PTI-801 (600 mg) and a mid-dose of PTI-808 (150 mg). Patients were first treated with PTI-808 or a placebo for seven days, followed by a 14-day triple combination treatment period, and then a seven-day washout period where no therapy was given. Patients who received the therapy combo with high-dose PTI-801 experienced significant improvement in lung function, with a 5% increase in mean absolute ppFEV1 at 14 days compared with before the treatment. These patients also showed significant reductions in sweat chloride levels, with a decrease of 19 mM at 14 days compared with before treatment, and 24 mM compared with placebo. Positive effects were also reported in patients who were predisposed to rapid pulmonary decline, and would not be included in other CFTR modulator combination studies. When treated with the combo regimen with 600 mg of PTI-801, these patients had an improvement in ppFEV1 of 6% compared with before treatment, and 8% compared with placebo.

ABBV-3067 (navocaftor, GLPG-3067, (5-(3-Amino-5-((4-(trifluoromethoxy)phenyl)sulfonyl)pyridin-2-yl)-1,3,4-oxadiazol-2-yl) methanol, CAS 2159103-66-7, e.g. WO2017/208115) is a CFTR potentiator which is being currently analyzed in a phase-II trial in combination with galicaftor as described above (NCT03969888). Previously, a phase-I study had been performed in healthy volunteers, consisting of a single ascending dose part receiving single oral doses ranging from 15 to 1000 mg of navocaftor and a multiple ascending dose part receiving oral doses starting from 45 mg once daily for 14 days, also in combination with galicaftor (NCT03128606). Navocaftor was generally well tolerated when dosed up to 1000 mg as single dose and up to 500 mg b.i.d. for 14 days in healthy subjects. Navocaftor exposure was not apparently altered in the presence of galicaftor and galicaftor exposure was similar in the presence of two different doses of navocaftor (Poster #36, North American Cystic Fibrosis Conference 2017, Indianapolis).

QBW-251 (icenticaftor, 3-Amino-6-methoxy-N-[(2S)-3,3,3-trifluoro-2-hydroxy-2-methylpropyl]-5-(trifluoromethyl)pyridine-2-carboxamide, CAS 1334546-77-8, e.g. WO2011/113894) is a CFTR potentiator which was tested in a placebo-controlled phase I/II study in a cohort of CF patients aged 18 years and older carrying CFTR Class class III, IV CFTR mutations on one allele, i.e. mutations where the CFTR is on the cell surface and can profit from potentiation (NCT02190604). One primary endpoint was change in lung clearance index after 14 days of treatment from baseline. Lung cleance index (LCI) is a measure of ventilation inhomogeneity that is derived from a multiple-breath washout test, and reduction in mean change from baseline for LCI2.5 of more than one unit indicates improvement. Other efficacy-related endpoints were absolute change from baseline in ppFEV1 and change from baseline in sweat chloride concentration, both after 14 days of treatment. Patients treated with 450 mg icenticaftor (b.i.d.) displayed an improved LCI2.5 (−1.13 compared to placebo), an improved ppFEV1 (6.5% increase versus placebo), and a decrease in sweat chloride concentration (−8.4 mM versus placebo) demonstrating CFTR target engagement and clinically meaningful improvement in lung function. In the same trial, and by contrast, the cohort of patients homozygous for the F508del-CFTR mutation treated with icenticaftor (450 mg b.i.d.) showed no reduction in sweat chloride or improvement in lung function indicating that combination with a CFTR corrector is required for this patient group (Kazani, 2021). In healthy volunteers icenticaftor was well tolerated at all exposures tested up to 1000 mg following SAD and up to 750 mg b.i.d. following MAD. 150 and 450 mg b.i.d. doses of icenticaftor (across all patient sub-groups) were well tolerated by CF patients with no unexpected events, deaths, or discontinuations (Kazani 2021).

Furthermore, icenticaftor was assessed in 92 patients aged 18 to 75 years with moderate to severe COPD and symptoms of chronic bronchitis in a placebo-controlled phase-II trial (NCT02449018). Patients received either 300 mg b.i.d. icenticaftor (N=64) or placebo (N=28) for 4 weeks on top of background COPD therapy. Primary endpoint was change from baseline of LCI2.5 after 4 weeks of treatment, key secondary endpoint was change from baseline in FEV1 after 4 weeks of treatment, and key exploratory endpoints included change from baseline of sweat chloride concentration and plasma fibrinogen concentration (a marker of inflammation) after 4 weeks of treatment. Icenticaftor treatment did not change LCI2.5 compared to placebo treatment. However, FEV1 was improved on icenticaftor treatment compared to placebo (+0.05-0.06 L, average baseline 1.4-1.5 L) as well as sweat chloride (−5.04 mM, average baseline 23 mM) and plasma fibrinogen (−0.39 g/L, average baseline 3.2 g/L) suggesting an improvement in several domains of COPD lung disease with icenticaftor treatment as compared to placebo (NCT02449018; Rowe, 2020). Currently, icenticaftor is being studied in a placebo-controlled phase-II clinical trial in subjects aged 18 years and older with bronchiectasis at an oral dose of 300 mg b.i.d. and a treatment duration of 12 weeks (NCT04396366). An estimated 72 patients are being randomized 1:1 to drug and placebo treatment. Primary endpoint is change from baseline after 1 weeks of treatment in bacterial load of potentially pathogenic microorganisms in sputum measured as colony forming units (CFU/mL). Key secondary endpoints measured after 12 weeks of treatment include change from baseline score in the Quality of Life Questionnaire for Bronchiectasis, respiratory domain only (QOL-B, a disease-specific questionnaire developed for non-cystic fibrosis bronchiectasis), change from baseline in fibrinogen plasma concentration, change in rescue medication use (salbutamol/albuterol), change from baseline in FEV1, change from baseline in airway lumen as measured by high resolution computed tomography (HRCT) and change from baseline in extent of global and regional air trapping in the airways as measured by HRCT.

VX-121 (vanzacaftor, (14S)-8-[3-(2-{Dispiro[2.0.2.1]heptan-7-yl}ethoxy)-1H-pyrazol-|-yl]-12,12-dimethyl-2\6-thia-3,9,11,18,23-pentaazatetracyclo[17.3.1.111,14.05,10]tetracosa-1(22),5,7,9,19(23),20-hexaene-2,2,4-trione, CAS 2374124-49-7, e.g. WO2019/161078, WO2021/030552) is a type-III CFTR corrector, that is currently being tested in two active-controlled double-blind, randomized phase-III clinical trials in CF patients as part of a new triple combination containing vanzacaftor, the type-I corrector tezacaftor and the CFTR potentiator VX-561 (deutivacaftor, a deuterated version of ivacaftor). In Trial 1 (NCT05033080) the new triple combination is compared to TRIKAFTA in an estimated 400 CF patients aged 12 and older which are heterozygous for the F508del-CFTR mutation and carry a minimal function mutation on the other allele. After a 4-week run-in period of dosing with TRIKAFTA, patients are randomized 1:1 to either continue on TRIKAFTA or to be dosed once daily with vanzacaftor, tezacaftor and deutivacaftor (20 mg/100 mg/250 mg) for 48 weeks. The primary efficacy endpoint is improvement in lung function as determined by the mean absolute change from baseline in ppFEV1 through 24 weeks of treatment. Key secondary endpoints determined at 24 weeks of treatment are absolute change from baseline in sweat chloride, proportion of participants with sweat chloride <60 mM and <30 mM from baseline through Week 24. In Trial 2 (NCT05076149) the new triple combination in a blinded fashion is compared to TRIKAFTA in an estimated 550 CF patients aged 12 and older which are homozygous for F508del-CFTR, or heterozygous for F508del-CFTR and a gating or residual function CFTR mutation, or have at least 1 other TRIKAFTA-responsive CFTR gene mutation and no F508del-CFTR mutation. After a 4-week run-in period of dosing with TRIKAFTA, patients are randomized 1:1 to either continue on TRIKAFTA or to be dosed once daily with vanzacaftor, tezacaftor and deutivacaftor (20 mg/100 mg/250 mg) for 48 weeks. The primary efficacy endpoint is improvement in lung function as determined by the mean absolute change from baseline in ppFEV1 through 24 weeks of treatment. Key secondary endpoints determined at 24 weeks of treatment are absolute change from baseline in sweat chloride, proportion of participants with sweat chloride <60 mM and <30 mM from baseline through Week 24.

The efficacy and safety of the new triple combination vanzacaftor/tezacaftor/deutivacaftor has previously been tested in a phase-II trial in CF patients (NCT03912233) either heterozygous for F508del and a minimal function mutation in the CFTR gene or in patients homozygous for the F508del-CFTR mutation. In the heterozygous patients (N=58), a once daily triple combination of vanzacaftor (5 or 10 or 20 mg)/tezacaftor 100 mg/deutivacaftor 150 mg was administered for 4 weeks and compared to placebo treatment followed by a 18-day wash-out from the triple combination onto the tezacaftor/deutivacaftor dual combination. The primary efficacy endpoint was improvement in lung function as determined by the mean absolute change from baseline in ppFEV1 through 4 weeks of treatment. The ppFEV1 treatment difference between the new triple combination and placebo was +2.7%/+12.3%/+7.9% in the arms with the 5 mg/10 mg/20 mg dose of vanzacaftor. The key secondary endpoint, absolute change from baseline in sweat chloride through 4 weeks of treatment, was favorably changed for the new triple combination with differences versus placebo of −45.1 mM/−48.1 mM/−51.8 mM in the arms with the 5 mg/10 mg/20 mg dose of vanzacaftor. In the homozygous patients (N=28) a 4-week run-in period of tezacaftor/ivacaftor (SYMKEVI) was followed by randomization to a continuation on tezacaftor/ivacaftor or a once daily dose of the new triple combination vanzacaftor 20 mg/tezacaftor 100 mg/deutivacaftor 150 mg for 4 weeks followed by a 28-day wash-out on the tezacaftor/ivacaftor dual combination. The primary efficacy endpoint was improvement in lung function as determined by the mean absolute change from baseline in ppFEV1 through 4 weeks of treatment. The ppFEV1 treatment difference between the new triple combination and dual combination was +16.0%. The key secondary endpoint, absolute change from baseline in sweat chloride through 4 weeks of treatment, was favorably changed for the new triple combination with differences versus dual combination of −42.9 mM. The new triple combination was generally well tolerated (Vertex Press Release 28 Jul. 2021).

VX-561 (Deutivacaftor, N-[2-tert-butyl-4-[1,1,1,3,3,3-hexadeuterio-2-(trideuteriomethyl) propan-2-yl]-5-hydroxyphenyl]-4-oxo-1H-quinoline-3-carboxamide, CTP-656, name, CAS 1413431-07-8, e.g. WO2019/109021, WO2019/018395) is a deuterated form of ivacaftor for once-daily application. A phase-II study (NCT03911713) compared different deutivacaftor doses with the standard ivacaftor dose (150 mg b.i.d=Kalydeco) in CF patients aged 18 and older with one of the following ivacaftor-responsive CFTR mutations on at least one allele: G551D, G178R, S549N, S549R, G551S, G1244E, S1251N, S1255P, or G1349D and who were already taking ivacaftor. Patients (N=77) were randomized to either continue on ivacaftor (150 mg b.i.d.) or to receive treatment with 25, 50, 150 or 250 mg of deutivacaftor once daily for a duration of 12 weeks. The primary efficacy endpoint was improvement in lung function as determined by the mean absolute change from baseline in ppFEV1 through 12 weeks of treatment. The ppFEV1 treatment difference for deutivacaftor versus ivacaftor was +3.9%/+3.5% in the arms with the 150 mg/250 mg once daily dose of deutivacaftor. The key secondary endpoint, absolute change from baseline in sweat chloride through 12 weeks of treatment, was determined at +2.4 mM and −7.4 mM for the deutivacaftor 150 mg/250 mg once daily arms when compared to ivacaftor 150 mg b.i.d. Mean c_{trough} plasma concentrations of deutivacaftor at 4 weeks of treatment were 458 ng/ml and 1100 ng/ml at the 150 mg and 250 mg doses while they were 952 ng/ml for the ivacaftor 150 mg b.i.d. dose.

The CFTR correctors lumacaftor, tezacaftor, galicaftor, Corr4a, elexacaftor, bamocaftor and others have been analyzed in vitro for their ability to restore folding of mutated CFTR (especially of F508del-CFTR) and its trafficking to the cell surface (Van Goor, 2011; Okiyoneda, 2013; Veit, 2018; Keating, 2018; Davies; 2018). Trafficking to the cell surface can be analyzed by different assay technologies including anti-CFTR immunoblotting (the CFTR “C band” represents the mature trafficked CFTR), anti-CFTR cell surface ELISAs, enzyme fragment complementation techniques or CFTR-HRP fusion proteins in recombinant cell lines expressing the mutated CFTR-protein or in primary cells from CF patients. Importantly, combinations of correctors with different types of corrector mechanism showed additive effects on increasing F508del-CFTR cell surface expression. This was shown for example for elexacaftor+/−tezacaftor (type-III corrector+/−type-I corrector; Keating, 2018; Veit, 2020) or bamocaftor+/−tezacaftor (type-III corrector+/−type-I corrector; Davies, 2018) or type-I+type-II+type-III correctors (Veit, 2018).

Mutated CFTR trafficked to the cell surface can still have a gating defect, and it has been shown that the function of cell surface F508del-CFTR is markedly augmented when a potentiator is added. Various functional assays are available to analyse CFTR function. For example, cellular expression of halide-sensitive yellow fluorescent protein can be used to measure iodide influx through functional CFTR (Galietta, 2001). Furthermore, electrophysiological measurements using the Ussing chamber and either recombinant epithelial cells or reconstituted bronchial epithelium from CF patients can be used to characterize the effects of CFTR modulators on CFTR function. For example, it was shown that the addition of the potentiator ivacaftor to lumacaftor-corrected F508del-CFTR augments CFTR function almost two-fold in reconstituted bronchial epithelium from CF patients (van Goor, 2011) in line with clinical trial data on FEV1 improvement upon ivacafor addition in lumacaftor-treated patients (Boyle, 2014). Similarly, again using Ussing chamber readouts, the additivity of type-I corrector tezacaftor and type-III corrector elexacaftor on functional correction of F508del-CFTR in reconstituted CF bronchial epithelium was shown as well as the further 2-fold augmentation of function by the addition of potentiator ivacaftor. Also, the additive effect of type-III corrector elexacaftor on a combination of type-I corrector tezacaftor and potentiator ivacaftor on F508del-CFTR function was demonstrated (Keating, 2018). The latter comparison is in line with clinical trial data in F508del-CFTR homozygous patients in which adding elexacaftor to a basal treatment of tezacaftor+ivacaftor (SYMDEKO) lead to a ppFEV1 increase of 10% (Keating, 2018). The FDA has recognized the predictive power of in vitro tests with respect to the effect of CFTR modulators in the clinic, and has approved CFTR modulators for rare CFTR-mutations based on in-vitro evidence only (Ussing chamber measurements in Fisher rat thyroid epithelial cells recombinantly expressing mutated CFTR; uspi SYMDEKO, uspi KALYDEKO, uspi TRIKAFTA).

Despite the availability of various CFTR modulators with complementary mechanism for many of the common mutations, CFTR function is not fully restored in patients (as indicated by the persistence of elevated sweat chloride levels) neither ist their lung function. These findings highlight the need for additional novel high efficacy CFTR corrector mechanisms that can be used alone or on top of currently available or future background therapy (i.e. SYMDEKO or ORKAMBI or TRIKAFTA or [galicaftor+navocaftor], and optionally further background therapy) and which may, in combination, increase the efficacy of such CFTR modulator therapy.

It has now been found that CFTR correctors such as especially the compounds of formula (I) as defined below, which are CFTR correctors with a novel mechanism (i.e. not type-I, not type-II, not type-III correctors) having potential in the prevention and treatment of CFTR-related diseases and disorders, especially of cystic fibrosis, may have complementary, and even synergistic effect when combined with CFTR correctors with another mechanism such as of type-I correctors (lumacaftor, tezacaftor, galicaftor), and/or type-II correctors (Corrector4a), and/or type-III correctors (elexacaftor, bamocaftor, olacaftor, vanzacaftor; and, in addition, ABBV-119, ABBV-567, and, in addition to the before-listed, PTI-801) and/or CFTR potentiators (ivacaftor, navocaftor, icenticaftor, deutivacaftor, GLPG-1837, GLPG2451) in the treatment of CFTR-related diseases and disorders, especially of cystic fibrosis. Such combination may, thus, especially be useful in the treatment of cystic fibrosis. Compounds of formula (I) can restore CFTR function in absence and presence of potentiators when determined by Ussing chamber measurements in reconstituted tissue from CF patients carrying the F508del-CFTR mutation.

In addition, CFTR correctors such as especially the compounds of formula (I) according to any one of the embodiments 1) to 15) may have complementary, and even synergistic effect when combined with CFTR stabilizers, such as SION-638 or NBD1-A, in the treatment of CFTR-related diseases and disorders, especially of cystic fibrosis. Such combination may, thus, especially be useful in the treatment of cystic fibrosis.

### DESCRIPTION OF THE FIGURES

FIG. 1 shows the effect of Example COMPOUND 3 on F508del-CFTR cell surface expression when given on top of basal treatments with type-I or type-II or type-III or [type-|+type-III] CFTR correctors.

FIG. 2 shows the effect of Example COMPOUND 3 on F508del-CFTR function (YFP quenching assay) when given on top of basal treatments with type-I CFTR corrector galicaftor and/or CFTR potentiator navocaftor.

FIG. 3 shows the effect of Example COMPOUND 3 on F508del-CFTR function (YFP quenching assay) when given on top of basal treatments with type-I CFTR corrector tezacaftor and/or CFTR potentiator ivacaftor.

FIG. 4 shows the effect of Example COMPOUND 3 on F508del-CFTR function (YFP quenching assay) when given on top of basal treatments with type-II CFTR corrector Corrector 4a and/or CFTR potentiator navocaftor.

FIG. 5 shows the effect of Example COMPOUND 3 on F508del-CFTR function (YFP quenching assay) when given on top of basal treatments with type-III CFTR corrector elexacaftor and/or CFTR potentiator ivacaftor.

FIG. 6 shows the effect of Example COMPOUND 3 on F508del-CFTR cell surface expression when given on top of basal treatments with type-III CFTR correctors elexacaftor or the compound of example 2 of WO 2019/071078 (PTI-801). In addition, the effect of adding one type-III corrector (elexacaftor) to another type-III corrector (PTI-801) is shown.

### DETAILED DESCRIPTION OF THE INVENTION

1) A first embodiment relates to a pharmaceutical composition comprising, as active principles, a compound of Formula (I):

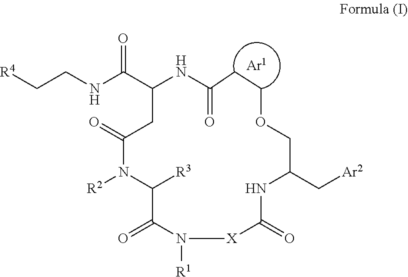

wherein
X represents —CR^{X1}R^{X2}, wherein R^{X1} represents hydrogen, and R^{X2} represents
   C₁₋₆-alkyl (especially methyl, ethyl, isopropyl, isobutyl);
   C₁₋₄-fluoroalkyl (especially 2,2,2-trifluoroethyl, 2,2-difluoroethyl); or
   C₃₋₆-cycloalkyl (especially cyclopentyl);
R¹ represents C₁₋₄-alkyl (especially methyl);
R² represents C₁₋₄-alkyl (especially methyl);
R³ represents C₁₋₆-alkyl (especially isobutyl);
R⁴ represents 5-membered heteroaryl (especially oxadiazolyl), wherein said 5-membered heteroaryl independently is unsubstituted, mono-, or di-substituted, wherein the substitutents are independently selected from C₁₋₄-alkyl (especially methyl), C₁₋₄-alkoxy (especially methoxy), C₁₋₃-fluoroalkyl, C₁₋₃-fluoroalkoxy, C₃₋₆-cycloalkyl (especially cyclopropyl), or halogen (especially fluoro);
Ar¹ represents 8- to 10-membered bicyclic heteroarylene (especially 10-membered bicyclic heteroarylene), wherein said bicyclic heteroarylene independently is unsubstituted or mono-substituted with C₁₋₄-alkyl (especially methyl), or halogen (especially fluoro);
Ar² represents phenyl, wherein said phenyl is unsubstituted, mono- or di-substituted wherein the substituents are independently selected from C₁₋₄-alkyl, C₁₋₃-fluoroalkyl, halogen, C₁₋₆-alkoxy, and C₁₋₃-fluoroalkoxy;
or a pharmaceutically acceptable salt thereof;
in combination with one or more therapeutically active ingredients acting as CFTR modulator(s); wherein said CFTR modulator(s) is/are one or more CFTR corrector(s) (especially a type-I corrector, and/or a type-II corrector, and/or a type-III corrector), and/or a CFTR potentiator; or a pharmaceutically acceptable salt thereof; as well as at least one pharmaceutically acceptable excipient.

The pharmaceutical composition according to embodiment 1) can be used as medicament, e.g. in the form of pharmaceutical compositions for enteral (such especially oral) or parenteral administration (including topical application or inhalation). The production of such pharmaceutical composition can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the combination active ingredients of the present invention, optionally in combination with other therapeutically valuable substances, into a galenical administration form together with suitable, non-toxic, inert, pharmaceutically acceptable solid or liquid carrier materials and, if desired, usual pharmaceutical adjuvants. A pharmaceutical composition for oral administration may in particular be in form of a capsule or tablet.

2) A second aspect relates to a pharmaceutical composition according to embodiment 1), wherein the compounds of Formula (I) are compounds of Formula (I_{E}):

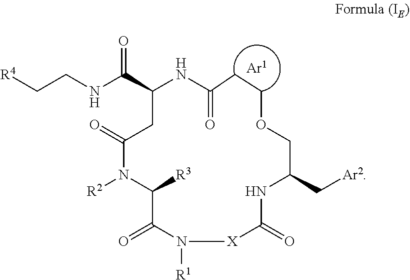

3) A further embodiment relates to a pharmaceutical composition according to embodiment 1) or 2), wherein X represents —CR^{X1}R^{X2}, wherein R^{X1} is hydrogen, and R^{X2} is C₁₋₄-fluoroalkyl (especially 2,2,2-trifluoroethyl, 2,2-difluoroethyl).

4) A further embodiment relates to a pharmaceutical composition according to embodiment 1) or 2), wherein X represents —CR^{X1}R^{X2}, wherein R^{X1} is hydrogen, and R^{X2} is 2,2,2-trifluoroethyl.

5) A further embodiment relates to a pharmaceutical composition according to any one of embodiments 1) to 4), wherein R¹ represents methyl.

6) A further embodiment relates to a pharmaceutical composition according to any one of embodiments 1) to 5), wherein R² represents methyl.

7) A further embodiment relates to a pharmaceutical composition according to any one of embodiments 1) to 6), wherein R³ is isobutyl.

8) A further embodiment relates to a pharmaceutical composition according to any one of embodiments 1) to 7), wherein R⁴ represents 5-membered heteroaryl (especially oxadiazolyl), wherein said 5-membered heteroaryl independently is mono-substituted with C₁₋₄-alkyl (especially methyl), C₁₋₄-alkoxy (especially methoxy), C₁₋₃-fluoroalkyl, C₁₋₃-fluoroalkoxy, or halogen (especially fluoro).

9) A further embodiment relates to a pharmaceutical composition according to any one of embodiments 1) to 7), wherein R⁴ represents 5-membered heteroaryl (especially oxadiazolyl), wherein said 5-membered heteroaryl independently is mono-substituted with C₁₋₄-alkoxy (especially methoxy).

10) A further embodiment relates to a pharmaceutical composition according to any one of embodiments 1) to 7), wherein R⁴ represents oxadiazolyl, wherein said 5-membered heteroaryl independently is mono-substituted with C₁₋₄-alkoxy (especially methoxy)

11) A further embodiment relates to a pharmaceutical composition according to any one of embodiments 1) to 10), wherein Ar¹ represents 8- to 10-membered bicyclic heteroarylene (especially 10-membered bicyclic heteroarylene), wherein said bicyclic heteroarylene independently is mono-substituted with halogen (especially fluoro).

12) A further embodiment relates to a pharmaceutical composition according to any one of embodiments 1) to 10), wherein Ar¹ represents 10-membered bicyclic heteroarylene (especially quinoline-diyl), wherein said bicyclic heteroarylene is mono-substituted with halogen (especially fluoro).

13) A further embodiment relates to a pharmaceutical composition according to any one of embodiments 1) to 12), wherein Ar² represents unsubstituted phenyl.

14) A further embodiment relates to a pharmaceutical composition according to embodiment 1), wherein the compound of Formula (I) is selected from the following compounds:
(3S,7S,10R,13R)-13-benzyl-7-isobutyl-N-(2-(3-methoxy-1,2,4-oxadiazol-5-yl)ethyl)-6,9,20-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10S,13R)-13-benzyl-7-isobutyl-N-(2-(3-methoxy-1,2,4-oxadiazol-5-yl)ethyl)-6,9,20-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10S,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-7-isobutyl-10-isopropyl-6,9-dimethyl-N-(2-(3-methylisoxazol-5-yl)ethyl)-1,5,8,11-tetraoxo-1,2,3,4,5,6,7,8,9,10,11,12,13,14-tetradecahydro-[1]oxa[4,7,10,14]tetraazacycloheptadecino[17,16-c]quinoline-3-carboxamide;
(3S,7S,10R,13R)-13-benzyl-7,10-diisobutyl-6,9-dimethyl-N-(2-(3-methylisoxazol-5-yl)ethyl)-1,5,8,11-tetraoxo-1,2,3,4,5,6,7,8,9,10,11,12,13,14-tetradecahydro-[1]oxa[4,7,10,14]tetraazacycloheptadecino[17,16-c]quinoline-3-carboxamide;
(3S,7S,10R,13R)-13-benzyl-7-isobutyl-6,9,10-trimethyl-N-(2-(3-methylisoxazol-5-yl)ethyl)-1,5,8,11-tetraoxo-1,2,3,4,5,6,7,8,9,10,11,12,13,14-tetradecahydro-[1]oxa[4,7,10,14]tetraazacycloheptadecino[17,16-c]quinoline-3-carboxamide;
(3S,7S,10R,13R)-13-benzyl-N-(2-(3-cyclopropylisoxazol-5-yl)ethyl)-7-isobutyl-6,9,10-trimethyl-1,5,8,11-tetraoxo-1,2,3,4,5,6,7,8,9,10,11,12,13,14-tetradecahydro-[1]oxa[4,7,10,14]tetraazacycloheptadecino[17,16-c]quinoline-3-carboxamide;
(3S,7S,10R,13R)-13-benzyl-10-(2,2-difluoroethyl)-7-isobutyl-6,9-dimethyl-N-(2-(3-methylisoxazol-5-yl)ethyl)-1,5,8,11-tetraoxo-1,2,3,4,5,6,7,8,9,10,11,12,13,14-tetradecahydro-[1]oxa[4,7,10,14]tetraazacycloheptadecino[17,16-c]quinoline-3-carboxamide;
(3S,7S,10R,13R)-13-benzyl-20-fluoro-7-isobutyl-N-(2-(3-methoxyisoxazol-5-yl)ethyl)-6,9,10-trimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-N-(2-(4-cyclopropyl-2H-1,2,3-triazol-2-yl)ethyl)-20-fluoro-7-isobutyl-6,9,10-trimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-7-isobutyl-N-(2-(3-methoxyisoxazol-5-yl)ethyl)-6,9,10-trimethyl-1,5,8,11-tetraoxo-1,2,3,4,5,6,7,8,9,10,11,12,13,14-tetradecahydroimidazo[11,2′:1,6]pyrido[2,3-p][1]oxa[4,7,10,14]tetraazacycloheptadecine-3-carboxamide;
(3S,7S,10R,13R)-13-benzyl-N-(2-(3-cyclopropylisoxazol-5-yl)ethyl)-7-isobutyl-6,9,10-trimethyl-1,5,8,11-tetraoxo-1,2,3,4,5,6,7,8,9,10,11,12,13,14-tetradecahydroimidazo[1′,2′:1,6]pyrido[2,3-p][1]oxa[4,7,10,14]tetraazacycloheptadecine-3-carboxamide;
(3S,7S,10R,13R)-13-benzyl-N-(2-(3-cyclopropylisoxazol-5-yl)ethyl)-20-fluoro-7-isobutyl-6,9,10-trimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-N-(2-(5-cyclopropylisoxazol-3-yl)ethyl)-20-fluoro-7-isobutyl-6,9,10-trimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-N-(2-(5-cyclopropyl-1,2,4-oxadiazol-3-yl)ethyl)-20-fluoro-7-isobutyl-6,9,10-trimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-N-(2-(3-cyclopropyl-1,2,4-oxadiazol-5-yl)ethyl)-20-fluoro-7-isobutyl-6,9,10-trimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-N-(2-(5-cyclopropyl-1,2,4-oxadiazol-3-yl)ethyl)-10-(2,2-difluoroethyl)-20-fluoro-7-isobutyl-6,9-dimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10S,13R)-13-benzyl-N-(2-(5-cyclopropyl-1,2,4-oxadiazol-3-yl)ethyl)-10-(2,2-difluoroethyl)-20-fluoro-7-isobutyl-6,9-dimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-N-(2-(5-cyclopropyl-1,2,4-oxadiazol-3-yl)ethyl)-10-ethyl-20-fluoro-7-isobutyl-6,9-dimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-10-(2,2-difluoroethyl)-20-fluoro-7-isobutyl-N-(2-(3-methoxyisoxazol-5-yl)ethyl)-6,9-dimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-7-isobutyl-6,9,10,20-tetramethyl-N-(2-(3-methylisoxazol-5-yl)ethyl)-1,5,8,11-tetraoxo-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-7-isobutyl-N-(2-(3-methoxyisoxazol-5-yl)ethyl)-6,9,10,20-tetramethyl-1,5,8,11-tetraoxo-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-N-(2-(3-cyclopropyl-1,2,4-oxadiazol-5-yl)ethyl)-7-isobutyl-6,9,10,20-tetramethyl-1,5,8,11-tetraoxo-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-N-(2-(5-cyclopropyl-2H-tetrazol-2-yl)ethyl)-10-(2,2-difluoroethyl)-20-fluoro-7-isobutyl-6,9-dimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-10-(2,2-difluoroethyl)-7-isobutyl-N-(2-(3-methoxyisoxazol-5-yl)ethyl)-6,9,20-trimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-10-(2,2-difluoroethyl)-7-isobutyl-N-(2-(3-methoxy-1,2,4-oxadiazol-5-yl)ethyl)-6,9,20-trimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-N-(2-(5-cyclopropyl-2H-tetrazol-2-yl)ethyl)-10-(2,2-difluoroethyl)-7-isobutyl-6,9,20-trimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-10-(2,2-difluoroethyl)-20-fluoro-7-isobutyl-6,9-dimethyl-N-(2-(3-methylisoxazol-5-yl)ethyl)-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-10-(2,2-difluoroethyl)-20-fluoro-7-isobutyl-N-(2-(3-methoxy-1,2,4-oxadiazol-5-yl)ethyl)-6,9-dimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-N-(2-(5-cyclopropyl-1,2,4-oxadiazol-3-yl)ethyl)-7-isobutyl-6,9,20-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-N-(2-(3-cyclopropyl-1,2,4-oxadiazol-5-yl)ethyl)-7-isobutyl-6,9,20-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-N-(2-(3-cyclopropylisoxazol-5-yl)ethyl)-7-isobutyl-6,9,20-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-7-isobutyl-N-(2-(3-methoxyisoxazol-5-yl)ethyl)-6,9,20-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-N-(2-(5-cyclopropylisoxazol-3-yl)ethyl)-7-isobutyl-6,9,20-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-N-(2-(5-cyclopropyl-2H-tetrazol-2-yl)ethyl)-7-isobutyl-6,9,20-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-N-(2-(4-fluoro-3-methoxyisoxazol-5-yl)ethyl)-7-isobutyl-6,9,20-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-10-(2,2-difluoroethyl)-20-fluoro-N-(2-(4-fluoro-3-methoxyisoxazol-5-yl)ethyl)-7-isobutyl-6,9-dimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-10-(2,2-difluoroethyl)-N-(2-(4-fluoro-3-methoxyisoxazol-5-yl)ethyl)-7-isobutyl-6,9,20-trimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-7-isobutyl-N-(2-(3-methoxy-1,2,4-oxadiazol-5-yl)ethyl)-6,9,17-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide;
(8R,11RS,14S,18S)-8-benzyl-14-isobutyl-N-(2-(3-methoxyisoxazol-5-yl)ethyl)-2,12,15-trimethyl-10,13,16,20-tetraoxo-11-(2,2,2-trifluoroethyl)-7,8,9,10,11,12,13,14,15,16,17,18,19,20-tetradecahydrooxazolo[4′,5′:5,6]benzo[1,2-p][1]oxa[4,7,10,14]tetraazacycloheptadecine-18-carboxamide;
(8R,11R,14S,18S)-8-benzyl-14-isobutyl-N-(2-(3-methoxyisoxazol-5-yl)ethyl)-2,12,15-trimethyl-10,13,16,20-tetraoxo-11-(2,2,2-trifluoroethyl)-7,8,9,10,11,12,13,14,15,16,17,18,19,20-tetradecahydrooxazolo[4′,5′:5,6]benzo[1,2-p][1]oxa[4,7,10,14]tetraazacycloheptadecine-18-carboxamide;
(8R,11RS,14S,18S)-8-benzyl-14-isobutyl-2,11,12,15-tetramethyl-N-(2-(3-methylisoxazol-5-yl)ethyl)-10,13,16,20-tetraoxo-7,8,9,10,11,12,13,14,15,16,17,18,19,20-tetradecahydrobenzofuro[7,6-p][1]oxa[4,7,10,14]tetraazacycloheptadecine-18-carboxamide;
(3S,7S,10R,13R)-13-benzyl-10-cyclopentyl-7-isobutyl-6,9-dimethyl-N-(2-(3-methylisoxazol-5-yl)ethyl)-1,5,8,11-tetraoxo-1,2,3,4,5,6,7,8,9,10,11,12,13,14-tetradecahydro-[1]oxa[4,7,10,14]tetraazacycloheptadecino[17,16-c]quinoline-3-carboxamide; and
(3S,7S,10R,13R)-13-benzyl-10-cyclopentyl-20-fluoro-7-isobutyl-6,9-dimethyl-N-(2-(3-methylisoxazol-5-yl)ethyl)-1,5,8,11-tetraoxo-1,2,3,4,5,6,7,8,9,10,11,12,13,14-tetradecahydro-[1]oxa[4,7,10,14]tetraazacycloheptadecino[17,16-c]quinoline-3-carboxamide.

15) A further embodiment relates to a pharmaceutical composition according to embodiment 1), wherein the compound of Formula (I) is
(3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide.

The corresponding structures of the compounds listed with their chemical names in embodiments 14) and 15) are shown in Table S, wherein, in case of doubt the depicted structure shall prevail.

Thus, for example the compound of example 43: (8R,11RS,14S,18S)-8-benzyl-14-isobutyl-2,11,12,15-tetramethyl-N-(2-(3-methylisoxazol-5-yl)ethyl)-10,13,16,20-tetraoxo-7,8,9,10,11,12,13,14,15,16,17,18,19,20-tetradecahydrobenzofuro[7,6-p][1]oxa[4,7,10,14]tetraazacycloheptadecine-18-carboxamide has the structure depicted in Table S, wherein regarding the chiral centers at carbon 8, 14, and 18, said compound is in absolute configuration as drawn; and with regard to the chiral center at carbon 11, the absolute configuration of said chiral center (marked as &1 may be both (R) or (S):

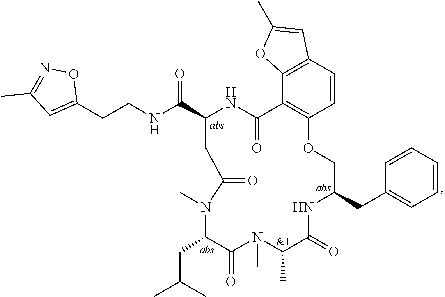 [SMILES: Cc1cc(CCNC(=O)[C@@H]2CC(=O)N(C)[C@@H](CC(C)C)C(=O)N(C)[C@@H](C)C(=O)N[C@H](Cc3ccccc3)COc3ccc4cc(C)oc4c3C(=O)N2)on1]

such compound encompassing the diastereomers:

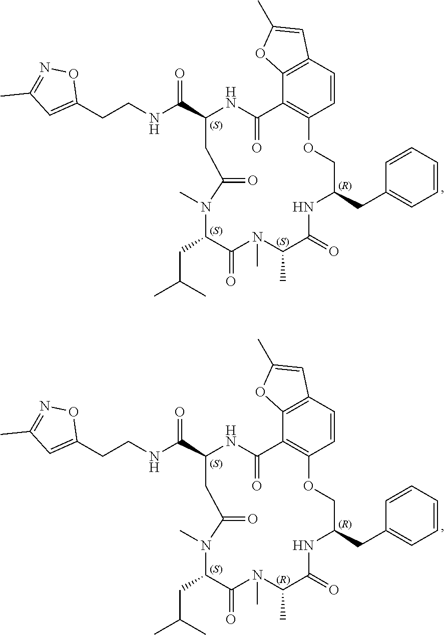

and any mixture thereof.

16) A further embodiment relates to a pharmaceutical composition according to any one of embodiments 1) to 15), wherein the one or more therapeutically active ingredients acting as CFTR modulator(s) is/are a type-I corrector selected from lumacaftor, tezacaftor, and galicaftor; and/or a type-II corrector which is Corrector4a; and/or a type-III corrector selected from elexacaftor, bamocaftor, olacaftor, and vanzacaftor; and/or a CFTR potentiator selected from ivacaftor, navocaftor, icenticaftor, deutivacaftor, GLPG-1837, and GLPG2451; or a pharmaceutically acceptable salt thereof.

17) A further embodiment relates to a pharmaceutical composition according to any one of embodiments 1) to 16), wherein the one or more therapeutically active ingredients acting as CFTR modulator(s) is/are a type-I corrector selected from lumacaftor, tezacaftor, and galicaftor; and/or a type-II corrector which is Corrector4a; and/or a type-III corrector selected from elexacaftor and vanzacaftor; and/or a CFTR potentiator selected from ivacaftor, navocaftor, icenticaftor, and deutivacaftor; or a pharmaceutically acceptable salt thereof.

18) A further embodiment relates to a pharmaceutical composition according to any one of embodiments 1) to 16), wherein the CFTR corrector is type-I corrector selected from lumacaftor, tezacaftor, and galicaftor.

19) A further embodiment relates to a pharmaceutical composition according to any one of embodiments 1) to 16), or 18), wherein the CFTR corrector is type-II corrector which is Corrector4a.

20) A further embodiment relates to a pharmaceutical composition according to any one of embodiments 1) to 16), 18), or 19), wherein the CFTR corrector is type-III corrector selected from elexacaftor, bamocaftor, olacaftor, and vanzacaftor.

21) A further embodiment relates to a pharmaceutical composition according to any one of embodiments 1) to 16), or 18) to 20), wherein a CFTR potentiator is selected from ivacaftor, navocaftor, icenticaftor, and deutivacaftor.

22) A further embodiment relates to a pharmaceutical composition according to any one of embodiments 1) to 17), wherein said composition comprises the compound of formula (I) and one therapeutically active ingredient acting as CFTR modulator, wherein said CFTR modulator is a CFTR potentiator; or a pharmaceutically acceptable salt thereof.

23) A further embodiment relates to a pharmaceutical composition according to any one of embodiments 1) to 17), or 22) wherein said composition comprises the compound of formula (I) and one therapeutically active ingredient acting as CFTR modulator, wherein said CFTR modulator is a CFTR potentiator selected from ivacaftor, icenticaftor, and deutivacaftor; or a pharmaceutically acceptable salt thereof.

24) A further embodiment relates to a pharmaceutical composition according to any one of embodiments 1) to 17), wherein said composition comprises the compound of formula (I) and one therapeutically active ingredient acting as CFTR modulator, wherein said CFTR modulator is a type-I corrector.

25) A further embodiment relates to a pharmaceutical composition according to any one of embodiments 1) to 17), wherein said composition comprises the compound of formula (I) and one therapeutically active ingredient acting as CFTR modulator, wherein said CFTR modulator is a type-II corrector.

26) A further embodiment relates to a pharmaceutical composition according to any one of embodiments 1) to 17), wherein said composition comprises the compound of formula (I) and one therapeutically active ingredient acting as CFTR modulator, wherein said CFTR modulator is a type-III corrector.

27) A further embodiment relates to a pharmaceutical composition according to any one of embodiments 1) to 17), wherein said composition comprises the compound of formula (I) and two therapeutically active ingredients acting as CFTR modulators, wherein one of said CFTR modulators is a CFTR potentiator or a pharmaceutically acceptable salt thereof, and, the second CFTR modulator is a CFTR corrector or a pharmaceutically acceptable salt thereof.

28) A further embodiment relates to a pharmaceutical composition according to any one of embodiments 1) to 17), or 27) wherein said composition comprises the compound of formula (I) and two therapeutically active ingredients acting as CFTR modulators, wherein one of said CFTR modulators is a CFTR potentiator or a pharmaceutically acceptable salt thereof, and, the second CFTR modulator is a type-I corrector or a pharmaceutically acceptable salt thereof.

29) A further embodiment relates to a pharmaceutical composition according to any one of embodiments 1) to 17), 27) or 28), wherein said composition comprises the compound of formula (I) and
ivacaftor and tezacaftor, or pharmaceutically acceptable salts thereof; or
ivacaftor and lumacaftor, or pharmaceutically acceptable salts thereof; or
navocaftor and galicaftor, or pharmaceutically acceptable salts thereof.

30) A further embodiment relates to a pharmaceutical composition according to any one of embodiments 1) to 17), or 27) to 29), wherein said composition comprises the compound of formula (I), navocaftor and galicaftor, or pharmaceutically acceptable salts thereof.

31) A further embodiment relates to a pharmaceutical composition according to any one of embodiments 1) to 17), or 27) to 29), wherein said composition comprises the compound of formula (I), ivacaftor and tezacaftor; or pharmaceutically acceptable salts thereof.

32) A further embodiment relates to a pharmaceutical composition according to any one of embodiments 1) to 17), wherein said composition comprises the compound of formula (I) and three therapeutically active ingredients acting as CFTR modulators, wherein one of said CFTR modulators is a CFTR potentiator or a pharmaceutically acceptable salt thereof, the second CFTR modulator is a type-I corrector or a pharmaceutically acceptable salt thereof, and the third CFTR modulator is a type-III corrector or a pharmaceutically acceptable salt thereof.

33) A further embodiment relates to a pharmaceutical composition according to embodiment 32), wherein said composition comprises the compound of formula (I) and
ivacaftor, tezacaftor and elexacaftor, or pharmaceutically acceptable salts thereof; or
deutivacaftor, tezacaftor and vanzacaftor, or pharmaceutically acceptable salts thereof.

34) A further embodiment relates to a pharmaceutical composition according to embodiment 31), wherein
ivacaftor, or a pharmaceutically acceptable salt thereof, is comprised in a pharmaceutical unit dosage suitable for the oral administration of ivacaftor, wherein ivacaftor, or a pharmaceutically acceptable salt thereof, is comprised in a pharmaceutical unit dosage suitable for the oral administration of a total of about 300 mg per day or below of ivacaftor; and
tezacaftor, or a pharmaceutically acceptable salt thereof, is comprised in a pharmaceutical unit dosage suitable for the oral administration of tezacaftor, wherein tezaftor, or a pharmaceutically acceptable salt thereof, is comprised in a pharmaceutical unit dosage suitable for the oral administration of a total of about 100 mg per day or below of tezaftor.

Such combination pharmaceutical compositions according to embodiments 1) to 34) are especially useful for the prevention or treatment of CFTR-related diseases as defined herein, in particular cystic fibrosis; and in a method for the prevention or treatment of CFTR-related diseases as defined herein, in particular cystic fibrosis, said method comprising administering a pharmaceutically efficacious dose of such combination pharmaceutical composition to a subject (especially a human) in need thereof.

Accordingly, the compound of formula (I) as defined in any one of embodiments 1) to 15), or a pharmaceutically acceptable salt thereof, according to this invention is for use in combination (or co-therapy) with said further pharmaceutically active ingredients as defined herein which are CFTR modulators (CFTR correctors and/or CFTR potentiators).

Definitions provided herein are intended to apply uniformly to any one of embodiments 1) to 43), and, mutatis mutandis, throughout the description and the claims unless an otherwise expressly set out definition provides a broader or narrower definition. It is well understood that a definition or preferred definition of a term defines and may replace the respective term independently of (and in combination with) any definition or preferred definition of any or all other terms as defined herein.

The term “therapeutically active ingredients acting as CFTR modulator” refers to any CFTR corrector (especially type-I−, type-II−, or type-III corrector) and/or CFTR potentiator that has shown-alone and/or in combination-potential for therapeutic use (as tested in in vitro and/or in vivo models, especially in clinical trials) and/or is indicated for such therapeutic use; wherein such therapeutic use is for CFTR-related disease (in particular cystic fibrosis). Examples are especially CFTR potentiators: ivacaftor, navocaftor, icenticaftor, deutivacaftor, GLPG-1837, and GLPG-2451; and CFTR correctors: type-I correctors (lumacaftor, tezacaftor, galicaftor), type-II correctors (Corrector4a), and type-III correctors (elexacaftor, bamocaftor, olacaftor, vanzacaftor; and, in addition ABBV-119, ABBV-567; and, in addition, PTI-801).

In a sub-embodiment, the present invention, thus, relates to the compounds of formula (I) as defined in any one of embodiments 1) to 15), or a pharmaceutically acceptable salt thereof, for use in the prophylaxis/prevention or treatment of CFTR-related diseases as defined herein, in particular cystic fibrosis; wherein said compound of formula (I) is (intended) to be administered/is administered in combination with CFTR potentiator, such as especially ivacaftor, navocaftor, icenticaftor, or deutivacaftor, or a pharmaceutically acceptable salt thereof; optionally additionally in combination with CFTR corrector, such as especially lumacaftor, tezacaftor, galicaftor, elexacaftor, or vanzacaftor, or a pharmaceutically acceptable salt thereof.

In addition, the present invention refers to pharmaceutical compositions comprising, as active principles, a compound of formula (I), or a pharmaceutically acceptable salt thereof, as defined according to any one of embodiments 1) to 15), in combination with one or more therapeutically active ingredients acting as CFTR modulator(s); wherein one or more of said CFTR modulator(s) is/are CFTR stabilizer(s), such as SION-638 or NBD1-A, or a pharmaceutically acceptable salt thereof; as well as at least one pharmaceutically acceptable excipient. CFTR stabilizer(s), such as SION-638 or NBD1-A, may additionally be implemented, mutatis mutandis, as CFTR modulator(s) in embodiments 16) to 43).

The term “subject” refers to a mammal, especially a human.

A combined treatment (or co-therapy) may notably be effected simultaneously (in a fixed dose or in a non-fixed dose).

“Simultaneously”, when referring to an administration type, means in the present application that the administration type concerned consists in the administration of two or more active ingredients and/or treatments at approximately the same time; wherein it is understood that a simultaneous administration will lead to exposure of the subject to the two or more active ingredients and/or treatments at the same time. When administered simultaneously, said two or more active ingredients may be administered in a fixed dose combination, or in a non-fixed dose combination, wherein such non-fixed combination may be a non-fixed dose combination equivalent to a fixed dose combination (e.g. by using two or more different pharmaceutical compositions to be administered by the same route of administration at approximately the same time), or a non-fixed dose combination using two or more different routes of administration or dosing regimens; wherein in each case said administration leads to essentially simultaneous exposure of the subject to the combined two or more active ingredients and/or treatments. An example of simultaneous administration of a non-fixed dose combination using two different pharmaceutical compositions to be administered by the same route of administration at approximately the same time is a non-fixed dose combination wherein the compound of formula (I) as defined in any one of embodiments 1) to 15) is administered b.i.d., and the respective CFTR modulator(s) is/are administered b.i.d. Another example of simultaneous administration of a non-fixed dose combination using two different routes of administration is a non-fixed dose combination wherein the compound of formula (I) as defined in any one of embodiments 1) to 15) is administered once a day or b.i.d., and the respective CFTR modulator(s) is/are administered t.i.d. Another example of simultaneous administration of a non-fixed dose combination using two different routes of administration is a non-fixed dose combination wherein the compound of formula (I) as defined in any one of embodiments 1) to 15) is administered once a day, and the respective CFTR modulator(s) is/are administered b.i.d. Another example of simultaneous administration of a non-fixed dose combination using two different routes of administration is a non-fixed dose combination wherein the compound of formula (I) as defined in any one of embodiments 1) to 15) is administered b.i.d., and the respective CFTR modulator(s) is/are administered once a day.

“Fixed dose combination”, when referring to an administration type, means in the present application that the administration type concerned consists in the administration of one single pharmaceutical composition comprising the two or more active ingredients, such as especially the pharmaceutical compositions of any one of embodiments 1) to 34).

35) Another aspect of the invention relates to the compound of formula (I) as defined in any one of embodiments 1) to 15), or a pharmaceutically acceptable salt thereof, for use in the treatment of CFTR-related diseases and disorders, especially of cystic fibrosis; wherein said compound is to be used/to be administered/administered in combination with one or more therapeutically active ingredients acting as CFTR modulator(s); wherein said CFTR modulator(s) is/are one or more CFTR corrector(s) (especially a type-I corrector, and/or a type-II corrector, and/or a type-III corrector), and/or a CFTR potentiator.

36) Another embodiment according to embodiment 35) relates to the compound of formula (I) as defined in any one of embodiments 1), 2) or 15), or to a pharmaceutically acceptable salt thereof, for use in the treatment of CFTR-related diseases and disorders, especially of cystic fibrosis; wherein said compound is to be used/to be administered/administered in combination with one or more therapeutically active ingredients acting as CFTR modulator(s); wherein said CFTR modulator(s) is/are one or more CFTR corrector(s) (especially a type-I corrector, and/or a type-II corrector, and/or a type-III corrector), and/or a CFTR potentiator.

37) Another embodiment relates to the compound of formula (I), or a pharmaceutically acceptable salt thereof for use according to embodiments 35) or 36), wherein said CFTR modulator(s) is/are as defined in any one of embodiments 22), 28), 29), 30), 31) or 33).

38) Another embodiment relates to the compound of formula (I) as defined in embodiment 15), for use according to embodiment 35) or 36), wherein said CFTR modulator(s) is/are as defined in any one of embodiments 22), 28), 29), 30), 31) or 33).

39) Another embodiment relates to the compound of formula (I) as defined in embodiment 15), for use according to embodiment 35) or 36), wherein said CFTR modulator(s) is/are as defined in embodiment 29).

40) Another embodiment relates to the compound of formula (I), which is (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide, or a pharmaceutically acceptable salt thereof, for use according to embodiment 35) or 36), wherein the CFTR modulators are navocaftor and galicaftor.

41) Another embodiment relates to the compound of formula (I), which is (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide, or a pharmaceutically acceptable salt thereof, for use according to embodiment 35) or 36), wherein the CFTR modulators are ivacaftor and tezacaftor.

42) Another aspect of the invention relates to a kit comprising
a pharmaceutical composition, as defined in any one of embodiments 1) to 34);
and instructions how to use said pharmaceutical composition for the treatment of CFTR-related diseases and disorders, especially of cystic fibrosis.

43) Another embodiment relates to a kit comprising
a pharmaceutical composition comprising the compound of formula (I) as defined in any one of embodiments 1) to 15);
one or more pharmaceutical composition(s) comprising one or more CFTR modulator(s) as defined in any one of embodiments 16) to 34);
and instructions how to use said pharmaceutical compositions in combination for the treatment of CFTR-related diseases and disorders, especially of cystic fibrosis.

It is understood that any embodiment relating to a compound of formula (I) as defined in any one of embodiments 1) to 15), or a pharmaceutically acceptable salt thereof, for combination use in the treatment of CFTR-related diseases as defined herein (especially of cystic fibrosis), wherein said compound of formula (I) is (intended) to be administered/is administered in combination with one or more CFTR modulator(s), wherein said CFTR modulators(s) is/are CFTR corrector(s) (especially a type-I−, a type-II−, a type-III-corrector) and/or a CFTR potentiator, or a pharmaceutically acceptable salt thereof, wherein said one or more CFTR modulator(s) are especially as defined in any one of embodiments 16) to 34), also relates
to said compound of formula (I) as defined in any one of embodiments 1) to 15), or a pharmaceutically acceptable salt thereof, for combination use in the treatment of CFTR-related diseases as defined herein (especially of cystic fibrosis), wherein said compound of formula (I) is (intended) to be administered/is administered in combination with said one or more CFTR modulator(s);
to said CFTR modulator(s) being CFTR corrector(s) and/or a CFTR potentiator, or a pharmaceutically acceptable salt thereof, for use in the treatment of said CFTR-related diseases (especially of cystic fibrosis); wherein said CFTR modulator(s) being CFTR corrector(s) and/or a CFTR potentiator, is (intended) to be administered in combination with said compound of formula (I);
to the use of said compound of formula (I), or of a pharmaceutically acceptable salt thereof, for the manufacture of a medicament/a pharmaceutical composition comprising said compound of formula (I), or a pharmaceutically acceptable salt thereof, and said CFTR modulator(s) being CFTR corrector(s) and/or a CFTR potentiator, or a pharmaceutically acceptable salt thereof, for use in the treatment of said CFTR-related diseases (especially of cystic fibrosis);
to the use of said compound of formula (I), or of a pharmaceutically acceptable salt thereof, for the manufacture of a medicament/pharmaceutical composition comprising, as active ingredient, said compound of formula (I), or a pharmaceutically acceptable salt thereof, for use in the treatment of said CFTR-related diseases (especially of cystic fibrosis); wherein said medicament/pharmaceutical composition is (intended) to be used in combination with said CFTR modulator(s) being CFTR corrector(s) and/or a CFTR potentiator, or a pharmaceutically acceptable salt thereof;
to the use of said CFTR modulator(s) being CFTR corrector(s) and/or a CFTR potentiator, or a pharmaceutically acceptable salt thereof, for the manufacture of a medicament/pharmaceutical composition comprising, as active ingredient, said CFTR modulator(s) being CFTR corrector(s) and/or a CFTR potentiator, or a pharmaceutically acceptable salt thereof, for use in the treatment of said CFTR-related diseases (especially of cystic fibrosis); wherein said medicament/pharmaceutical composition is (intended) to be used in combination with said compound of formula (I), or a pharmaceutically acceptable salt thereof;
to the use of a pharmaceutical composition comprising said compound of formula (I), or a pharmaceutically acceptable salt thereof, and said CFTR modulator(s) being CFTR corrector(s) and/or a CFTR potentiator, or a pharmaceutically acceptable salt thereof, for the treatment of said CFTR-related diseases (especially of cystic fibrosis);
to a medicament for use in the prevention or treatment of said CFTR-related diseases (especially of cystic fibrosis), said medicament comprising said compound of formula (I), or a pharmaceutically acceptable salt thereof; wherein said medicament is (intended) to be administered in combination with said CFTR modulator(s) being CFTR corrector(s) and/or a CFTR potentiator, or a pharmaceutically acceptable salt thereof;
to a method of preventing or treating said CFTR-related diseases (especially of cystic fibrosis) comprising administering to a subject (preferably a human) in need thereof an effective amount of said compound of formula (I), or a pharmaceutically acceptable salt thereof, wherein said compound of formula (I) is administered in combination with an effective amount of said CFTR modulator(s) being CFTR corrector(s) and/or a CFTR potentiator, or of a pharmaceutically acceptable salt thereof; wherein it is understood that said combined administration may be in a fixed-dose combination or in a non-fixed dose combination;
to a method of preventing or treating said CFTR-related diseases (especially of cystic fibrosis) comprising administering to a subject in need thereof an effective amount of a pharmaceutical composition comprising said compound of formula (I), or a pharmaceutically acceptable salt thereof, and said CFTR modulator(s) being CFTR corrector(s) and/or a CFTR potentiator, or a pharmaceutically acceptable salt thereof; and
to a method of preventing or treating said CFTR-related diseases (especially of cystic fibrosis) comprising administering to a subject (preferably a human) in need thereof an effective amount of said CFTR modulator(s) being CFTR corrector(s) and/or a CFTR potentiator, or of a pharmaceutically acceptable salt thereof, wherein said anti-CFTR modulator(s) being CFTR corrector(s) and/or a CFTR potentiator, is administered in combination with an effective amount of said compound of formula (I), or of a pharmaceutically acceptable salt thereof wherein it is understood that said combined administration may be in a fixed-dose combination or in a non-fixed dose combination.

CFTR-related diseases and disorders may be defined as including especially cystic fibrosis, as well as further CFTR-related diseases and disorders selected from:
chronic bronchitis; rhinosinusitis; constipation; pancreatitis; pancreatic insufficiency; male infertility caused by congenital bilateral absence of the vas deferens (CBAVD); mild pulmonary disease; allergic bronchopulmonary aspergillosis (ABPA); liver disease; coagulation-fibrinolysis deficiencies, such as protein C deficiency; and diabetes mellitus;
asthma; COPD; smoke induced COPD; and dry-eye disease; and
idiopathic pancreatitis; hereditary emphysema; hereditary hemochromatosis; lysosomal storage diseases such as especially I-cell disease pseudo-Hurler; mucopolysaccharidoses; Sandhoff/Tay-Sachs; osteogenesis imperfecta; Fabry disease; Sjogren's disease; osteoporosis; osteopenia; bone healing and bone growth (including bone repair, bone regeneration, reducing bone resorption and increasing bone deposition); chloride channelopathies, such as myotonia congenita (Thomson and Becker forms); Bartter's syndrome type 3; epilepsy; lysosomal storage disease; Primary Ciliary Dyskinesia (PCD)—a term for inherited disorders of the structure and or function of cilia (including PCD with situs inversus also known as Kartagener syndrome, PCD without situs inversus, and ciliary aplasia); generalized epilepsy with fibrile seizures plus (GEFS+); general epilepsy with febrile and afebrile seizures; myotonia; paramyotonia congenital; potassium-aggravated myotonia; hyperkalemic periodic paralysis; long QT syndrome (LQTS); LQTS/Brugada syndrome; autosomal-dominant LQTS with deafness; autosomal-recessive LQTS; LQTS with dysmorphic features; congenital and acquired LQTS; dilated cardiomyopathy; autosomal-dominant LQTS; osteopetrosis; and Bartter syndrome type 3.

The term “treatment of cystic fibrosis” refers to any treatment of cystic fibrosis and includes especially treatment that reduces the severity of cystic fibrosis and/or reduces the symptoms of cystic fibrosis.

The term “cystic fibrosis” refers to any form of cystic fibrosis, especially to a cystic fibrosis that is associated with one or more gene mutation(s). Preferably, such cystic fibrosis is associated with an CFTR trafficking defect (class II mutations) or reduced CFTR stability (class VI mutations) [in particular, an CFTR trafficking defect/class II mutation], wherein it is understood that such CFTR trafficking defect or reduced CFTR stability may be associated with another disease causing mutation of the same or any other class. Such further disease causing CFTR gene mutation comprises class I mutations (no functional CFTR protein), (a further) class II mutation (CFTR trafficking defect), class III mutations (CFTR regulation defect), class IV mutations (CFTR conductance defect), class V mutations (less CFTR protein due to splicing defects), and/or (a further) class VI mutation (less CFTR protein due to reduced CFTR stability). Said one or more gene mutation(s) may for example comprise at least one mutation selected from F508del, A561E, and N1303K, as well as 1507del, R560T, R1066C and V520F; in particular F508del. In addition to the above-listed, further CFTR gene mutations comprise for example G85E, R347P, L206W, and M1101K. Said gene mutation(s) may be heterozygous, homozygous or compound hetereozygous. Especially said gene mutation is heterozygous comprising one F508del mutation. Further CFTR gene mutations (which are especially class III and/or IV mutations) comprise G551D, R117H, D1152H, A455E, S549N, R347H, S945L, and R117C.

The severity of cystic fibrosis/of a certain gene mutation associated with cystic fibrosis as well as the efficacy of correction thereof may generally be measured by testing the chloride transport effected by the CFTR. In patients, for example average sweat chloride content may be used for such assessment.

The term “symptoms of cystic fibrosis” refers especially to elevated chloride concentration in the sweat; symptoms of cystic fibrosis further comprise chronic bronchitis; rhinosinusitis; constipation; pancreatitis; pancreatic insufficiency; male infertility caused by congenital bilateral absence of the vas deferens (CBAVD); mild pulmonary disease; allergic bronchopulmonary aspergillosis (ABPA); liver disease; coagulation-fibrinolysis deficiencies such as protein C deficiency; and/or diabetes mellitus.

Where the plural form is used for compounds, salts, pharmaceutical compositions, diseases and the like, this is intended to mean also a single compound, salt, or the like.

Any reference to compounds is to be understood as referring also to the salts (and especially the pharmaceutically acceptable salts) of such compounds, as appropriate and expedient.

The term “pharmaceutically acceptable salts” refers to salts that retain the desired biological activity of the subject compound and exhibit minimal undesired toxicological effects. Such salts include inorganic or organic acid and/or base addition salts depending on the presence of basic and/or acidic groups in the subject compound. For reference see for example “Handbook of Pharmaceutical Salts. Properties, Selection and Use.”, P. Heinrich Stahl, Camille G. Wermuth (Eds.), Wiley-VCH, 2008; and “Pharmaceutical Salts and Co-crystals”, Johan Wouters and Luc Quéré (Eds.), RSC Publishing, 2012.

Definitions provided herein are intended to apply uniformly to the compounds of formula (I)/formula (I_{E}), as defined in any one of embodiments 1) to 15), and, mutatis mutandis, throughout the description and the claims unless an otherwise expressly set out definition provides a broader or narrower definition. It is well understood that a definition or preferred definition of a term defines and may replace the respective term independently of (and in combination with) any definition or preferred definition of any or all other terms as defined herein. Whenever a substituent is denoted as optional, it is understood that such substituent may be absent (i.e. the respective residue is unsubstituted with regard to such optional substituent), in which case all positions having a free valency (to which such optional substituent could have been attached to; such as for example in an aromatic ring the ring carbon atoms and/or the ring nitrogen atoms having a free valency) are substituted with hydrogen where appropriate. Likewise, in case the term “optionally” is used in the context of (ring) heteroatom(s), the term means that either the respective optional heteroatom(s), or the like, are absent (i.e. a certain moiety does not contain heteroatom(s)/is a carbocycle/or the like), or the respective optional heteroatom(s), or the like, are present as explicitly defined.

The term “halogen” means fluorine/fluoro, chlorine/chloro, or bromine/bromo; preferably fluorine/fluoro.

The term “alkyl”, used alone or in combination, refers to a saturated straight or branched chain hydrocarbon group containing one to six carbon atoms. The term “C_{x-y}-alkyl” (x and y each being an integer), refers to an alkyl group as defined before, containing x to y carbon atoms. For example, a C₁₋₆-alkyl group contains from one to six carbon atoms. Representative examples of alkyl groups are methyl, ethyl, propyl, isopropyl, butyl, isobutyl, tert.-butyl, 3-methyl-butyl, 2,2-dimethyl-propyl and 3,3-dimethyl-butyl. For avoidance of any doubt, in case a group is referred to as e.g. propyl or butyl, it is meant to be n-propyl, respectively n-butyl. In case R¹ represents a C₁₋₄-alkyl group, the term especially refers to methyl. For R² representing C₁₋₄-alkyl the term especially means methyl. In case R³ represents —C₁₋₆-alkyl, the term especially means isobutyl.

The term “alkoxy”, used alone or in combination, refers to an alkyl-O— group wherein the alkyl group is as defined before. The term “C_{x-y}-alkoxy” (x and y each being an integer) refers to an alkoxy group as defined before containing x to y carbon atoms. For example, a C₁₋₄-alkoxy group means a group of the formula C₁₋₄-alkyl-O— in which the term “C₁₋₄-alkyl” has the previously given significance. Representative examples of alkoxy groups are methoxy, ethoxy, n-propoxy, isopropoxy, n-butoxy, isobutoxy, sec-butoxy and tert-butoxy. Preferred is methoxy.

The term “fluoroalkyl”, used alone or in combination, refers to an alkyl group as defined before containing one to three carbon atoms in which one or more (and possibly all) hydrogen atoms have been replaced with fluorine. The term “C_{x-y}-fluoroalkyl” (x and y each being an integer) refers to a fluoroalkyl group as defined before containing x to y carbon atoms. For example, a C₁₋₃-fluoroalkyl group contains from one to three carbon atoms in which one to seven hydrogen atoms have been replaced with fluorine. Representative examples of fluoroalkyl groups include especially C₁-fluoroalkyl groups such as trifluoromethyl, and difluoromethyl, as well as 2-fluoroethyl, 2,2-difluoroethyl and 2,2,2-trifluoroethyl. In case R^{X2} represents C₁₋₄-fluoroalkyl, the term especially means 2,2-difluoroethyl or 2,2,2-trifluoroethyl.

The term “fluoroalkoxy”, used alone or in combination, refers to an alkoxy group as defined before containing one to three carbon atoms in which one or more (and possibly all) hydrogen atoms have been replaced with fluorine. The term “C_{x-y}-fluoroalkoxy” (x and y each being an integer) refers to a fluoroalkoxy group as defined before containing x to y carbon atoms. For example, a C₁₋₃-fluoroalkoxy group contains from one to three carbon atoms in which one to seven hydrogen atoms have been replaced with fluorine. Representative examples of fluoroalkoxy groups include trifluoromethoxy, difluoromethoxy, 2-fluoroethoxy, 2,2-difluoroethoxy and 2,2,2-trifluoroethoxy. Preferred are (C₁) fluoroalkoxy groups such as trifluoromethoxy and difluoromethoxy.

The term “cycloalkyl”, used alone or in combination, refers to a saturated monocyclic hydrocarbon ring containing three to six carbon atoms. The term “C_{x-y}cycloalkyl” (x and y each being an integer), refers to a cycloalkyl group as defined before containing x to y carbon atoms. For example, a C₃₋₆-cycloalkyl group contains from three to six carbon atoms. Examples of cycloalkyl groups are cyclopropyl, cyclobutyl, cyclopentyl, and cyclohexyl.

The term “heteroaryl”, used alone or in combination, and if not explicitly defined in a broader or more narrow way, means a 5- to 10-membered monocyclic or bicyclic aromatic ring containing one to a maximum of four heteroatoms, each independently selected from oxygen, nitrogen and sulfur. Representative examples of such heteroaryl groups are 5-membered heteroaryl groups such as furanyl, oxazolyl, isoxazolyl, oxadiazolyl, thiophenyl, thiazolyl, isothiazolyl, thiadiazolyl, pyrrolyl, imidazolyl, pyrazolyl, triazolyl, tetrazolyl; 6-membered heteroaryl groups such as pyridinyl, pyrimidinyl, pyridazinyl, pyrazinyl; and 8- to 10-membered bicyclic heteroaryl groups such as indolyl, isoindolyl, benzofuranyl, isobenzofuranyl, benzothiophenyl, indazolyl, benzimidazolyl, benzoxazolyl, benzisoxazolyl, benzothiazolyl, benzoisothiazolyl, benzotriazolyl, benzoxadiazolyl, benzothiadiazolyl, thienopyridinyl, quinolinyl, isoquinolinyl, naphthyridinyl, cinnolinyl, quinazolinyl, quinoxalinyl, phthalazinyl, pyrrolopyridinyl, pyrazolopyridinyl, pyrazolopyrimidinyl, pyrrolopyrazinyl, imidazopyridinyl, imidazopyridazinyl, and imidazothiazolyl. The above-mentioned heteroaryl groups are unsubstituted or substituted as explicitly defined.

For the substituent R⁴ representing a “5-membered heteroaryl”, the term especially means isoxazolyl, oxadiazolyl, triazolyl, tetrazolyl; notably isoxazol-3-yl, isoxazol-5-yl, 1,2,4-oxadiazol-5-yl, 1,2,4-oxadiazol-3-yl, 2H-[1,2,3]triazol-2-yl, 2H-tetrazol-2-yl; in particular 1,2,4-oxadiazol-5-yl. Said R⁴ group is unsubstituted or substituted as explicitly defined.

For the substituent Ar¹ representing “8- to 10-membered bicyclic heteroarylene”, the term especially means benzofuran-diyl, benzoxazole-diyl, imidazopyridine-diyl, quinoline-diyl, isoquinoline-diyl; notably benzofuran-6,7-diyl, benzo[d]oxazole-6,7-diyl, imidazo[1,2-a]pyridine-5,6-diyl, quinoline-3,4-diyl, quinoline-5,6-diyl, isoquinoline-5,6-diyl; in particular quinoline-5,6-diyl. Said Ar¹ group is unsubstituted or substituted as explicitly defined.

The compounds of formula (I) contain at least three stereogenic or asymmetric centers, which are present in (R)- or (S)-configuration as defined in the respective embodiment defining such compound of formula (I). In addition, the compounds of formula (I) may contain one or more further stereogenic or asymmetric centers, such as one or more additional asymmetric carbon atoms. The compounds of formula (I) may thus be present as mixtures of stereoisomers or preferably as pure stereoisomers. Mixtures of stereoisomers may be separated in a manner known to a person skilled in the art. In case any stereogenic or asymmetric center in a given chemical name is designated as being in (RS)-configuration, this means that such stereogenic or asymmetric center in such compound may be present in (R)-configuration, in(S)-configuration, or in any mixture of epimers with regard to such center.

Thus, for example the compound (8R,11RS,14S,18S)-8-benzyl-14-isobutyl-N-(2-(3-methoxyisoxazol-5-yl)ethyl)-2,12,15-trimethyl-10,13,16,20-tetraoxo-11-(2,2,2-trifluoroethyl)-7,8,9,10,11,12,13,14,15,16,17,18,19,20-tetradecahydrooxazolo[4′,5′:5,6]benzo[1,2-p][1]oxa[4,7,10,14]tetraazacycloheptadecine-18-carboxamide comprises (8R,11R,14S,18S)-8-benzyl-14-isobutyl-N-(2-(3-methoxyisoxazol-5-yl)ethyl)-2,12,15-trimethyl-10,13,16,20-tetraoxo-11-(2,2,2-trifluoroethyl)-7,8,9,10,11,12,13,14,15,16,17,18,19,20-tetradecahydrooxazolo[4′,5′:5,6]benzo[1,2-p][1]oxa[4,7,10,14]tetraazacycloheptadecine-18-carboxamide; the compound (8R,11S,14S,18S)-8-benzyl-14-isobutyl-N-(2-(3-methoxyisoxazol-5-yl)ethyl)-2,12,15-trimethyl-10,13,16,20-tetraoxo-11-(2,2,2-trifluoroethyl)-7,8,9,10,11,12,13,14,15,16,17,18,19,20-tetradecahydrooxazolo[4′,5′:5,6]benzo[1,2-p][1]oxa[4,7,10,14]tetraazacycloheptadecine-18-carboxamide, and any mixture thereof. Likewise, in a certain chemical structure (such as in Table S), a stereogenic or asymmetric center indicated as “abs” represents said stereogenic or asymmetric center in the respective (R)- or (S)-configuration. A stereogenic or asymmetric center indicated as “&1” represents said stereogenic or asymmetric center in the respective (RS)-configuration, i.e. comprising the respective (R)- or (S)-configuration or any mixture of epimers at such center.

Unless used regarding temperatures, the term “about” placed before a numerical value “X” refers in the current application to an interval extending from X minus 10% of X to X plus 10% of X, and preferably to an interval extending from X minus 5% of X to X plus 5% of X. In the particular case of temperatures, the term “about” placed before a temperature “Y” refers in the current application to an interval extending from the temperature Y minus 10° C. to Y plus 10° C., preferably to an interval extending from Y minus 5° C. to Y plus 5° C., notably to an interval extending from Y minus 3° C. to Y plus 3° C. Room temperature means a temperature of about 25° C. When in the current application the term n equivalent(s) is used wherein n is a number, it is meant and within the scope of the current application that n is referring to about the number n, preferably n is referring to the exact number n.

Whenever the word “between” or “to” is used to describe a numerical range, it is to be understood that the end points of the indicated range are explicitly included in the range. For example: if a temperature range is described to be between 40° C. and 80° C. (or 40° C. to 80° C.), this means that the end points 40° C. and 80° C. are included in the range; or if a variable is defined as being an integer between 1 and 4 (or 1 to 4), this means that the variable is the integer 1, 2, 3, or 4.

The term “essentially”, is understood in the context of the present invention to mean especially that the respective amount/purity/time etc. is at least 90, especially at least 95, and notably at least 99 percent of the respective total. For example when used in the term “essentially simultaneous exposure” is understood to mean especially that the respective exposure results in simultaneous exposure of pharmaceutically effective amounts of all combination active ingredients during at least 90, especially at least 95, and notably at least 99 percent of the time, i.e. of the day in case chronic/steady state exposure to the pharmaceutically active ingredients is contemplated.

Particular embodiments of the invention are described in the following examples, which serve to illustrate the invention in more detail without limiting its scope in any way.

### EXPERIMENTAL PART

### I. Chemistry

All temperatures are stated in ° C. Commercially available starting materials were used as received without further purification. Unless otherwise specified, all reactions were carried out in oven-dried glassware under an atmosphere of nitrogen. Compounds were purified by flash column chromatography on silica gel or by preparative HPLC. Compounds described in the invention are characterised by LC-MS data (retention time t_{R} is given in min; molecular weight obtained from the mass spectrum is given in g/mol) using the conditions listed below. In cases where compounds of the present invention appear as a mix. of conformational isomers, particularly visible in their LC-MS spectra, the retention time of the most abundant conformer is given.

### Analytical LC-MS Equipment:

HPLC pump: Binary gradient pump, Agilent G4220A or equivalent
Autosampler: Gilson LH215 (with Gilson 845z injector) or equivalent
Column compartment: Dionex TCC-3000RS or equivalent
Degasser: Dionex SRD-3200 or equivalent
Make-up pump: Dionex HPG-3200SD or equivalent
DAD detector: Agilent G4212A or equivalent
MS detector: Single quadrupole mass analyzer, Thermo Finnigan MSQPlus or equivalent
ELS detector: Sedere SEDEX 90 or equivalent
   LC-MS with Acidic Conditions
Method A: Column: Waters Atlantis T3 (3.0 μm, 2.1×50 mm). Conditions: MeCN+0.1% formic acid [eluent A]; water+0.1% formic acid [eluent B]. Gradient: 95% B→2% B over 5 min (flow 0.8 mL/min). Detection: UV/Vis+MS.
Method B: Column: Zorbax RRHD SB-aq (1.8 μm, 2.1×50 mm). Conditions: MeCN [eluent A]; water+0.04% TFA [eluent B]. Gradient: 95% B→5% B over 2.0 min (flow: 0.8 mL/min). Detection: UVNVis+MS.
Method C: Column: Waters XSelect CSH C18 (3.5 μm, 2.1×30 mm). Conditions: MeCN+0.1% formic acid [eluent A]; water+0.1% formic acid [eluent B]. Gradient: 95% B→2% B over 1.6 min (flow 1 mL/min), Detection: UV/Vis+MS.
Method D: Column: Waters BEH C18 (2.1×50 mm, 2.5 μm). Conditions: MeCN [eluent A]; water+0.04% TFA [eluent B]. Gradient: 95% B→5% B over 2.0 min (flow: 0.8 mL/min). Detection: UV/Vis+MS.
Method E: Waters Acquity Binary, Solvent Manager, MS: Waters SQ Detector or Xevo TQD or SYNAPT G2 MS, DAD: Acquity UPLC PDA Detector, ELSD: Acquity UPLC ELSD. Column ACQUITY UPLC CSH C18 1.7 μm 2.1×50 mm from Waters, thermostated in the Acquity UPLC Column Manager at 60° C. Eluents: A: H₂O+0.05% formic acid; B: MeCN+0.045% formic acid. Method: Gradient: 2% B→98% B over 2.0 min. Flow: 1.0 mL/min. Detection: UV 214 nm and ELSD, and MS, t_{R} is given in min.
   LC-MS with Basic Conditions
Method F: Column: Waters BEH C18 (2.5 μm, 2.1×50 mm). Conditions: water/NH₃ [c(NH₃)=13 mmol/l] [eluent A]; MeCN [eluent B]. Gradient: 5% B→95% B over 2 min (flow 0.8 mL/min). Detection: UV/Vis+MS.
Method G: Column: Waters XSelect CSH C18 (3.5 μm, 2.1×30 mm). Conditions: 95% MeCN+5% Water/NH₄HCO₃ [c(NH₄HCO₃)=10 mmol/l] [eluent A]; Water/NH₄HCO₃ [c(NH₄HCO₃)=10 mmol/l] [eluent B]. Gradient: 95% B→2% B over 1.6 min (flow 1 mL/min), Detection: UV/Vis+MS.

### Preparative HPLC Equipment:

Gilson 333/334 HPLC pump equipped with Gilson LH215, Dionex SRD-3200 degasser,
Dionex ISO-3100A make-up pump, Dionex DAD-3000 DAD detector, Single quadrupole mass analyzer MS detector, Thermo Finnigan MSQ Plus, MRA100-000 flow splitter, Polymer Laboratories PL-ELS1000 ELS detector
   Preparative HPLC with Basic Conditions
Column: Waters XBridge (10 μm, 75×30 mm). Conditions: MeCN [eluent A]; water+0.5% NH₄OH (25% aq.) [eluent B]; Gradient see Prep. HPLC Table 1 (flow: 75 mL/min), the starting percentage of Eluent A (x) is determined depending on the polarity of the compound to purify. Detection: UV/Vis+MS

TABLE 1
Prep. HPLC

| t (min) | 0 | 0.01 | 4.0 | 6.0 | 6.2 | 6.6 |
|---|---|---|---|---|---|---|
| Eluent A (%) | x | x | 95 | 95 | x | x |
| Eluent B (%) | 100-x | 100-x | 5 | 5 | 100-x | 100-x |

Preparative HPLC with Acidic Conditions
Column: Waters Atlantis T3 (10 μm, 75×30 mm). Conditions: MeCN [eluent A]; water+0.5% HCO₂H [eluent B]; Gradient see Prep. HPLC Table 2 (flow: 75 mL/min), the starting percentage of Eluent A (x) is determined depending on the polarity of the compound to purify. Detection: UV/Vis+MS

TABLE 2
Prep. HPLC

| t (min) | 0 | 0.01 | 4.0 | 6.0 | 6.2 | 6.6 |
|---|---|---|---|---|---|---|
| Eluent A (%) | x | x | 95 | 95 | x | x |
| Eluent B (%) | 100-x | 100-x | 5 | 5 | 100-x | 100-x |

### Preparative HPLC for Chiral Separations

In most cases, desired diastereoisomers can be isolated or purified by standard preparative scale HPLC according to standard methods well-known to those skilled in the art. In some instances, the use of a chiral chromatography column is advisable to separate complex mixtures of diastereoisomers. Best results are obtained using Chiral Stationary Phase columns, such as Chiralpak IA, IB, or IC columns based on an immobilised amylose or cellulose chiral phase, with an isocratic eluent based on a mix. of MeCN with EtOH or MeOH, in a ratio varying from 9:1 to 1:9. In order to compensate for the presence of ionisable functional groups in the compound being purified, modifiers can be added to the solvent mix. such as 0.1% diethylamine for basic derivatives or 0.1% formic acid for acidic ones. Supercritical Fluid Chromatography was used in some cases, using the same Chiral Stationary Phase columns as described above with isocratic eluents composed of 50% to 90% supercritical carbondioxide together with EtOH, MeOH or a 1:1 EtOH:MeCN mix. Detection: UV/Vis.

### Abbreviations (as Used Hereinbefore or Hereinafter)

AcOH acetic acid
aq. aqueous
atm atmosphere
BnBr benzyl bromide
Boc tert-butoxycarbonyl
Boc₂O di-tert-butyl dicarbonate
nBuLi n-butyllithium
CHCl₃ chloroform
d days
DCM dichloromethane
DIAD diisopropyl azodicarboxylate
DIPEA diisopropyl-ethylamine, Hünig's base
DMF dimethylformamide
DMSO dimethylsulfoxide
DPPA diphenyl phosphorylazide
dppf 1,1′-bis(diphenylphosphino) ferrocene
Et ethyl
Et₂O diethylether
EtOAc ethyl acetate
EtOH ethanol
evaporated evaporated in vacuo
Ex. example
FC flash chromatography on silica gel
Fmoc 9-Fluorenylmethoxycarbonyl
h hour(s)
HATU (1-[Bis(dimethylamino)methylene]-1H-1,2,3-triazolo[4,5-b]pyridinium 3-oxid hexafluorophosphate
hept heptane(s)
hex hexane(s)
HPLC high performance liquid chromatography
HV high vacuum conditions
LC-MS liquid chromatography-mass spectrometry
Lit. Literature
M mol/l
Me methyl
MeCN acetonitrile
Mel iodomethane
Meldrum's acid 2,2-Dimethyl-1,3-dioxane-4,6-dione
MeOH methanol
mL milliliter
min minute(s)
mix. mixture
MOM methoxymethyl
MW microwave
NaBH(OAc)₃ sodium triacetoxyborohydride
NCS N-chlorosuccinimide
NMP N-methyl-2-pyrrolidone
OAc acetate
org. organic
Pd(^{t}Bu₃P)₂ bis(tri-tert-butylphosphine) palladium (0)
Pd(OAc)₂ palladium (II) acetate
Pd/C palladium on activated charcoal
Pd(OH)₂/C palladium hydroxide on activated charcoal (Pearlman's catalyst)
Pd₂(dba)₃ tris(dibenzylideneacetone) dipalladium (0)
Pd(PPh₃)₄ tetrakis(triphenylphosphine) palladium (0)
Ph phenyl
PhMe toluene
PPh₃ triphenyl phosphine
prep. preparative
PyClop Chlorotripyrrolidinophosphonium hexafluorophosphate
rac racemic
RM reaction mix.
RT room temperature
S second(s)
sat. saturated
Selectfluor 1-chloromethyl-4-fluoro-1,4-diazoniabicyclo[2.2.2]octane bis(tetrafluoroborate)
SM starting material
soln. solution
tBu tert-butyl=tertiary butyl
TEA triethylamine
Tf trifluoromethanesulfonyl
TFA trifluoroacetic acid
THF tetrahydrofuran
T3P n-propylphosphonic anhydride
t_{R} retention time
uM micromolar
XPhos 2-dicyclohexylphosphin-2′,4′,6′-triisopropylbiphenyl

### Preparation of Precursors and Intermediates

### Amines:

Commercially available amines are depicted in Table AM.

TABLE AM

| Amine | Name |
|---|---|
| AM1 | 2-(5-Cyclopropyl-1,2,4-oxadiazol-3-yl)ethan-1-amine•HCl |
| AM2 | 2-(5-Cyclopropyl-1,2-oxazol-3-yl)ethan-1-amine |
| AM3 | 2-(3-Cyclopropyl-1,2,4-oxadiazol-5-yl)ethan-1-amine•HCl |
| AM4 | 2-(3-Methyl-1,2-oxazol-5-yl)ethan-1-amine•HCl |

Non-commercial amines are synthesised as described below.

### 2-(3-Cyclopropylisoxazol-5-yl) ethan-1-amine hydrochloride (AM5)

Step 1: A soln. of DIAD (61.7 mL, 318 mmol) in THF (350 mL) is added dropwise to a 0° C. soln. of but-3-yn-1-ol (22.9 mL, 318 mmol), isoindoline-1,3-dione (44.5 g, 302 mmol) and PPh₃ (83 g, 318 mmol) in THF (1500 mL) and the RM is stirred for 1 h. The RM is concentrated in vacuo and the residue is dissolved in hot PhMe (370 mL) before MeOH (210 mL) is slowly added. The RM is cooled to RT and MeOH is added until a white solid precipitates. The RM is partially concentrated before the solid is collected by filtration washing with cold PhMe and then air dried to give 2-(but-3-yn-1-yl) isoindoline-1,3-dione. LC-MS C: t_{R}=1.81 min; No ionisation.

Step 2: Na₂CO₃ (22.7 g, 214 mmol) is carefully added to a RT soln. of hydroxylamine·HCl (37.2 g, 535 mmol) in H₂O (125 mL) followed by the slow addition of a soln. of cyclopropanecarbaldehyde (26.7 mL, 357 mmol) in EtOH (100 mL). The RM is stirred for 1 h and then partitioned between H₂O and EtOAc and extracted. The layers are separated and the aq. phase is re-extracted with EtOAc (2×). The combined org. layers are washed with brine, dried over Na₂SO₄, filtered and evaporated in vacuo to give the crude product that is recrystallised from n-hept to give E/Z-cyclopropanecarbaldehyde oxime as a white solid. LC-MS C: t_{R}=2.13 & 2.30 min; No ionisation.

Step 3: NCS (34.3 g, 257 mmol) is added portion wise to a 0° C. soln. of cyclopropanecarbaldehyde oxime (19.3 g, 226 mmol) and pyridine (0.83 mL, 10.3 mmol) in DMF (100 mL) and the RM is stirred for 3 h. A soln. of 2-(but-3-yn-1-yl) isoindoline-1,3-dione (21.0 g, 103 mmol) in DMF (100 mL) followed by TEA (28.7 mL, 206 mmol) are added and the RM is stirred for 3 h. The RM is partitioned between H₂O and DCM and extracted. The layers are separated and the aq. phase is re-extracted with DCM (2×). The combined org. layers are washed with brine, dried over Na₂SO₄, filtered and evaporated in vacuo to give the crude product that is triturated with MeOH to give 2-(2-(3-cyclopropylisoxazol-5-yl)ethyl) isoindoline-1,3-dione as a white solid. LC-MS G: t_{R}=1.91 min; [M+H]⁺=283.1.

Step 4: Hydrazine·H₂O (9.44 mL, 194 mmol) is added to a RT suspension of 2-(2-(3-cyclopropylisoxazol-5-yl)ethyl) isoindoline-1,3-dione (28.0 g, 97 mmol) in EtOH (100 mL) and the RM is heated to 80° C. for 5 h. The RM is cooled to RT and filtered washing with EtOH. The filtrate is concentrated in vacuo and the residue is suspended in Et₂O and re-filtered washing with Et₂O. The filtrate is partially concentrated before 1M HCl in Et₂O (100 mL) is added and the precipitate is filtered and dried in vacuo to give the title compound as a white solid. LC-MS G: t_{R}=1.42 min; [M+H]⁺=153.1.

### 2-(5-Cyclopropyl-2H-tetrazol-2-yl) ethan-1-amine hydrochloride (AM6)

Step 1: tert-Butyl N-(2-bromoethyl) carbamate (2.22 g, 9.7 mmol) is added to a RT suspension of 5-cyclopropyl-2H-1,2,3,4-tetrazole (1.00 g, 8.81 mmol) and K₂CO₃ (1.46 g, 10.6 mmol) in MeCN (20 mL) and the RM is heated to 50° C. for 4 h. The RM is allowed to reach RT, then MeCN is evaporated before water and EtOAc are added. The layers are separated and the aq. phase re-extracted with EtOAc (2×). The combined org. layers are dried (MgSO₄), filtered, and evaporated. Purification by FC (eluting with 20% to 80% EtOAc in hept) yields tert-butyl (2-(5-cyclopropyl-2H-tetrazol-2-yl)ethyl) carbamate (1.21 g, 55%) as a colourless oil. LC-MS F: t_{R}=0.80 min; [M+H]⁺=254.35.

Step 2: 4 M HCl in dioxane (12 mL, 48.1 mmol) is added to a RT solution of tert-butyl (2-(5-cyclopropyl-2H-tetrazol-2-yl)ethyl) carbamate (1.21 g, 4.81 mmol) in dioxane (26 mL) and the RM is stirred at RT for 46 h. The RM is concentrated and co-evaporated with Et₂O in vacuo to obtain the title compound (0.92 g, 100%) as a white solid. LC-MS F: t_{R}=0.44 min; [M+H]⁺=154.25.

### 2-(3-Methoxy-1,2,4-oxadiazol-5-yl) ethan-1-amine hydrochloride (AM7)

Step 1: HATU (11.82 g, 31.1 mmol) is added to a RT solution of boc-beta-Ala-OH (5.0 g, 25.9 mmol), o-methylisourea bisulfate (4.5 g, 25.9 mmol, and DIPEA (18.1 mL, 104 mmol) in DMF (150 mL) and the RM is stirred at RT for 1.5 h. Water and EtOAc are added to the RM, then the two layers are separated and the inorg. layer is extracted with EtOAc (2×). The combined org. layers are washed with brine, dried (Na₂SO₄), filtered, and concentrated to give the crude product that is purified by FC (eluting with 20% to 100% EtOAc in hept) to give tert-butyl (3-((imino(methoxy)methyl)amino)-3-oxopropyl) carbamate as a white solid. LC-MS F: t_{R}=0.64 min; [M+H]⁺=246.36.

Step 2: 1,8-Diazabicyclo[5.4.0]undec-7-ene (8.96 mL, 59.3 mmol) is added to a RT soln. of tert-butyl (3-(imino(methoxy)methyl)amino)-3-oxopropyl) carbamate (6.19 g, 24.7 mmol) and NBS (10.56 g, 59.3 mmol) in EtOAc (120 mL) and the RM is stirred for 5 h. Additional 1,8-diazabicyclo[5.4.0]undec-7-ene (1.85 mL, 12.4 mmol) and NBS (2.2 g, 12.4 mmol) are added and stirring is continued for 16 h. The suspension is filtered and the filtrate is washed with H₂O, sat. aq. NaHCO₃ soln. and brine before being evaporated to dryness. The crude product is purified by FC (eluting with 20% to 100% EtOAc in hept) to give tert-butyl (2-(3-methoxy-1,2,4-oxadiazol-5-yl)ethyl) carbamate as a colourless oil. LC-MS F: t_{R}=0.75 min; [M+H]⁺=244.33.

Step 3: 4 M HCl in dioxane (0.62 mL, 2.47 mmol) is added to a RT solution of tert-butyl (2-(3-methoxy-1,2,4-oxadiazol-5-yl)ethyl) carbamate (150 mg, 0.62 mmol) in DCM (2 mL) and the RM is stirred for 4 days at RT, then at 50° C. for 6 h. The mixture is evaporated to yield the title compound (79 mg, 71%) as a white solid. LC-MS F: t_{R}=0.35 min; [M+H]⁺=144.21.

### 2-(4-Cyclopropyl-2H-1,2,3-triazol-2-yl) ethan-1-amine hydrochloride (AM8)

Step 1: Pd(OAc)₂ (17.1 mg, 0.076 mmol) is added to a RT solution of tert-butyl N-[2-(4-bromo-2H-1,2,3-triazol-2-yl)ethyl]carbamate (291 mg, 1 mmol), cyclopropylboronic acid (112 mg, 1.3 mmol), potassium phosphate tribasic (758 mg, 3.5 mmol), and tricyclohexylphosphine (45.1 mg, 0.156 mmol) in toluene (22 mL) and H₂O (0.22 mL). The mix. is heated to 100° C. for 18 h. The RM is allowed to cool down to RT, then the mixture is filtered, and the filtrate concentrated. Purification by FC (eluting with 5% to 40% EtOAc in hept with R_{f}=0.27 in hept/EtOAc 7:3) yields tert-butyl (2-(4-cyclopropyl-2H-1,2,3-triazol-2-yl)ethyl) carbamate (194 mg, 77%) as a yellow oil. LC-MS B: t_{R}=0.82 min; [M+H]⁺=253.34.

Step 2: 4 M HCl in dioxane (4.9 mL, 19.6 mmol) is added to a RT solution of tert-butyl (2-(4-cyclopropyl-2H-1,2,3-triazol-2-yl)ethyl) carbamate (550 mg, 1.96 mmol) in DCM (3.4 mL). The RM is stirred at RT for 30 min, then the RM is concentrated to give title compound (432 mg, 98%) as a white solid which is used as such in the next step. LC-MS B: t_{R}=0.37 min; [M+H]⁺=153.11.

### 2-(3-Methoxyisoxazol-5-yl) ethan-1-amine hydrochloride (AM9)

Step 1: DPPA (1.32 mL, 6.1 mmol) is added dropwise to a RT soln. of 3-(3-methoxyisoxazol-5-yl) propanoic acid (1.0 g, 5.55 mmol) and TEA (0.93 mL, 6.66 mmol) in PhMe (25 mL) and the RM is heated to 100° C. for 1.5 h. 2-Methylpropan-2-ol (1.06 mL, 11.1 mmol) is added and the RM is heated to reflux for 16 h. The RM is cooled to RT and partitioned between sat. aq. NaHCO₃ and EtOAc and the layers are separated. The aq. phase is re-extracted with EtOAc (2×) and the combined org. extracts are washed with brine, dried over Na₂SO₄, filtered and evaporated in vacuo. The crude product is purified by FC (eluting with 0% to 100% EtOAc in hept) to give tert-butyl (2-(3-methoxyisoxazol-5-yl)ethyl) carbamate as a colourless oil. LC-MS C: t_{R}=1.80 min; [M+H]⁺=243.1.

Step 2: The title compound is prepared from tert-butyl (2-(3-methoxyisoxazol-5-yl)ethyl) carbamate in analogy to the procedure described for AM8, step 2. LC-MS B: t_{R}=0.28 min; [M+H]⁺=143.09.

### 2-(4-Fluoro-3-methoxyisoxazol-5-yl) ethan-1-amine hydrochloride (AM10)

Step 1: In a microwave tube, phthalic anhydride (354 mg, 2.36 mmol) is added to a RT suspension of AM9 (402 mg, 2.25 mmol) and DIPEA (0.47 mL, 2.7 mmol) in dioxane (12 mL). The tube is sealed and heated to 100° C. for 48 h. Water is added to the RM, the mixture is acidified with 1 M HCl and the product extracted with EtOAc, dried (MgSO₄), filtered, and concentrated to yield 2-(2-(3-methoxyisoxazol-5-yl)ethyl) isoindoline-1,3-dione (718 mg) as a white solid which was used as such in the next step. LC-MS B: t_{R}=0.85 min; [M+H]⁺=273.09.

Step 2: Selectfluor (1.07 g, 2.87 mmol) is added to a 40° C. solution of 2-(2-(3-methoxyisoxazol-5-yl)ethyl) isoindoline-1,3-dione (710 mg, 2.61 mmol) in tetramethylene sulfone (21.7 mL, 226 mmol) and the RM is heated to 120° C. for 18 h. The resulting dark brown solution is allowed to cool down to around 50° C., then the RM is poured into pre-stirred H₂O (30 mL), followed by EtOAc (10 mL). The two layers are separated and the inorg. layer is extracted with EtOAc (5 mL). The comb. org. layers are washed with brine, dried (Na₂SO₄), filtered, and concentrated. Purification by prep HPLC (acidic) yields 2-(2-(4-fluoro-3-methoxyisoxazol-5-yl)ethyl) isoindoline-1,3-dione (89 mg) as a colourless oil. LC-MS B: t_{R}=0.90 min; [M+H]⁺=291.02.

Step 3: Hydrazine monohydrate (0.222 mL, 2.93 mmol) is added to a RT solution of 2-(2-(4-fluoro-3-methoxyisoxazol-5-yl)ethyl) isoindoline-1,3-dione (85 mg, 0.293 mmol) in EtOH (3 mL) and the RM is heated to 80° C. for 1 h. The RM is cooled down to RT and a white precipitate is formed. Ether is added and the solid is triturated before filtered off. The filtrate is concentrated to yield title compound (40 mg) as a colourless oil which is used as such in the next step. LC-MS B: t_{R}=0.33 min; [M+H]⁺=161.08.

### Preparation of Building Blocks A

### tert-Butyl(S)-3-amino-4-((2-(3-methylisoxazol-5-yl)ethyl)amino)-4-oxobutanoate (A1)

Step 1: HATU (11.10 g, 29.2 mmol) is added to a RT soln. of Fmoc-L-aspartic acid beta-tert-butyl ester (12.26 g, 29.20 mmol) and DIPEA (7.5 mL, 43.8 mmol) in DMF (100 mL). After stirring for 5 min at RT, a solution of 2-(3-methylisoxazol-5-yl) ethan-1-amine hydrochloride (AM4, 5.00 g, 29.2 mmol) and DIPEA (7.5 mL, 43.8 mmol) in DMF (100 mL) is added, and stirring is continued for 30 min. The RM is partitioned between H₂O and EtOAc and the layers are separated. The aq. layer is re-extracted with EtOAc (2×) and the combined org. layers are washed with brine, dried (MgSO₄), filtered, and evaporated to yield tert-butyl(S)-3-((((9H-fluoren-9-yl)methoxy)carbonyl)amino)-4-((2-(3-methylisoxazol-5-yl)ethyl)amino)-4-oxobutanoate as a yellow oil which is used as such in the next step. LC-MS F: t_{R}=1.10 min; [M+H]⁺=520.26.

Step 2: Piperidine (15.0 mL, 150.0 mmol) is added to a RT soln. of tert-butyl(S)-3-((((9H-fluoren-9-yl)methoxy)carbonyl)amino)-4-((2-(3-methylisoxazol-5-yl)ethyl)amino)-4-oxobutanoate (17.9 g, max 29.2 mmol) in DCM (200 mL) and the RM is stirred for 30 min. The RM is concentrated and the residue directly purified by FC (eluting with 100:2:0.5 DCM:MeOH:NH₃) to give the title compound (4.02 g) as a yellowish solid. LC-MS F: t_{R}=0.64 min; [M+H]⁺=298.26.

Listed in Table A below are building blocks A that are prepared in analogy to the 2-step sequence described above for A1.

TABLE A

| | | | t_{R} [min] | MS-data |
|---|---|---|---|---|
| | | | LC-MS | m/z |
| No. | Starting Material | Product | Method | [M + H]⁺ |
| A2 | 2-(5-Cyclopropylisoxazol-3- | tert-Butyl (S)-3-amino-4-((2-(5- | 0.76 | 324.27 |
| | yl)ethan-1-amine (AM2) | cyclopropylisoxazol-3-yl)ethyl)amino)-4- | F | |
| | | oxobutanoate | | |
| A3 | 2-(5-Cyclopropyl-1,2,4- | tert-Butyl (S)-3-amino-4-((2-(5- | 0.70 | 325.31 |
| | oxadiazol-3-yl)ethan-1-amine | cyclopropyl-1,2,4-oxadiazol-3- | F | |
| | hydrochloride (AM1) | yl)ethyl)amino)-4-oxobutanoate | | |
| A4 | 2-(3-Cyclopropyl-1,2,4- | tert-Butyl (S)-3-amino-4-((2-(3- | 0.72 | 325.29 |
| | oxadiazol-5-yl)ethan-1-amine | cyclopropyl-1,2,4-oxadiazol-5- | F | |
| | hydrochloride (AM3) | yl)ethyl)amino)-4-oxobutanoate | | |
| A5 | 2-(3-Cyclopropylisoxazol-5- | tert-Butyl (S)-3-amino-4-((2-(3- | 0.73 | 324.31 |
| | yl)ethan-1-amine | cyclopropylisoxazol-5-yl)ethyl)amino)-4- | F | |
| | hydrochloride (AM5) | oxobutanoate | | |
| A6 | 2-(3-Methoxyisoxazol-5- | tert-Butyl (S)-3-amino-4-((2-(3- | 0.68 | 314.36 |
| | yl)ethan-1-amine | methoxyisoxazol-5-yl)ethyl)amino)-4- | F | |
| | hydrochloride (AM9) | oxobutanoate | | |
| A7 | 2-(3-Methoxy-1,2,4- | tert-Butyl (S)-3-amino-4-((2-(3-methoxy- | 0.62 | 315.44 |
| | oxadiazol-5-yl)ethan-1-amine | 1,2,4-oxadiazol-5-yl)ethyl)amino)-4- | F | |
| | hydrochloride (AM7) | oxobutanoate | | |
| A8 | 2-(5-Cyclopropyl-2H-tetrazol- | tert-Butyl (S)-3-amino-4-((2-(5- | 0.96 | 325.47 |
| | 2-yl)ethan-1-amine | cyclopropyl-2H-tetrazol-2- | F | |
| | hydrochloride (AM6) | yl)ethyl)amino)-4-oxobutanoate | | |
| A9 | 2-(4-Cyclopropyl-2H-1,2,3- | tert-Butyl (S)-3-amino-4-((2-(4- | 0.59 | 324.03 |
| | triazol-2-yl)ethan-1-amine | cyclopropyl-2H-1,2,3-triazol-2- | B | |
| | hydrochloride (AM8) | yl)ethyl)amino)-4-oxobutanoate | | |
| A10 | 2-(4-Fluoro-3- | tert-Butyl (S)-3-amino-4-((2-(4-fluoro-3- | 0.71 | 332.50 |
| | methoxyisoxazol-5-yl)ethan- | methoxyisoxazol-5-yl)ethyl)amino)-4- | F | |
| | 1-amine hydrochloride | oxobutanoate | | |
| | (AM10) | | | |

### Allyl(S)-3-amino-4-((2-(3-methylisoxazol-5-yl)ethyl)amino)-4-oxobutanoate hydrochloride (A11)

Step 1: HATU (6.80 g, 17.9 mmol) is added to a RT soln. of Boc-L-aspartic acid-beta-allyl ester (4.80 g, 17 mmol) mmol), 2-(3-methyl-1,2-oxazol-5-yl) ethan-1-amine hydrochloride (AM4, 3.00 g, 17.5 mmol), and DIPEA (11.7 mL, 43.8 mmol) in DMF/DCM (1/1) mix. (20 mL) and the RM is stirred for 18 h. The mixture is concentrated and the crude purified by FC (100% DCM to DCM/MeOH 9/1), followed by prep. HPLC (acidic) to yield allyl (S)-3-((tert-butoxycarbonyl)amino)-4-((2-(3-methylisoxazol-5-yl)ethyl)amino)-4-oxobutanoate (3.76 g). LC-MS B: t_{R}=0.83 min; [M+H]⁺=382.41.

Step 2: 4M HCl in dioxane (4.87 mL, 39.5 mmol) is added to a soln. of allyl (S)-3-((tert-butoxycarbonyl)amino)-4-((2-(3-methylisoxazol-5-yl)ethyl)amino)-4-oxobutanoate (3.76 g) in DCM (15 mL) and the RM is stirred for 2 h at RT. The volatiles are removed in vacuo and the residue is triturated with Et₂O to give the title compound as a white solid. LC-MS F: t_{R}=0.57 min; [M+H]⁺=282.21.

### Preparation of Building Blocks B

### Benzyl (R)-3-(2-amino-3-phenylpropoxy) quinoline-4-carboxylate dihydrochloride (B1)

Step 1: KHCO₃ (5.88 g, 58.1 mmol) and BnBr (7.69 mL, 63.4 mmol) are added to a soln. of 3-hydroxyquinoline-4-carboxylic acid (10.0 g, 52.9 mmol) in DMF (100 mL), and the RM is stirred for 16 h. The RM is filtered and the filtrate concentrated in vacuo. The residue is partitioned between H₂O and EtOAc. The layers are separated and the aq. layer is re-extracted with EtOAc (2×). The combined org. layers are washed with brine, dried (MgSO₄), filtered, and evaporated. The crude product is purified by FC (eluting with 20% to 80% EtOAc in hept) to give benzyl 3-hydroxyquinoline-4-carboxylate (6.97 g) as a white solid. LC-MS B: t_{R}=0.95 min; [M+H]⁺=280.19.

Step 2: DIAD (1.9 mL, 9.47 mmol) is added to a 0° C. mix. of benzyl 3-hydroxyquinoline-4-carboxylate (1.47 g, 5.26 mmol), tert-butyl (R)-(1-hydroxy-3-phenylpropan-2-yl) carbamate (2.02 g, 7.89 mmol), and PPh₃ (2.51 g, 9.47 mmol) in THF (22 mL) and the RM is stirred for 16 h at RT. The mix. is concentrated and the residue directly purified by FC (eluting with 20% to 50% EtOAc in hept) to give benzyl (R)-3-(2-((tert-butoxycarbonyl)amino)-3-phenylpropoxy) quinoline-4-carboxylate (2.55 g) as a white solid. LC-MS B: t_{R}=1.17 min; [M+H]⁺=513.06.

Step 3: 4M HCl in dioxane (12.5 mL, 49.8 mmol) is added to a soln. of benzyl (R)-3-(2-((tert-butoxycarbonyl)amino)-3-phenylpropoxy) quinoline-4-carboxylate (2.55 g, 4.98 mmol) in dioxane (11.5 mL) and the RM is stirred for 1 h at 50° C. The volatiles are removed in vacuo and the residue is triturated with Et₂O (3×) to give the title compound as a yellowish solid. LC-MS B: t_{R}=0.83 min; [M+H]⁺=413.34.

### Methyl (R)-6-(2-amino-3-phenylpropoxy)-2-methylbenzo[d]oxazole-7-carboxylate hydrochloride (B2)

Step 1: Nitric acid (0.36 mL, 6.0 mmol) is carefully added to a 0° C. soln. of methyl 2,6-dihydroxybenzoate (1.0 g, 6.0 mmol) in acetic acid (10 mL) and the RM is warmed to RT and stirred for 1 h. The RM is poured into cold water and the precipitate collected by filtration and washed with additional cold water before being dried in vacuo to give methyl 2,6-dihydroxy-3-nitrobenzoate as a pink solid. LC-MS B: t_{R}=0.75 min; No ionisation. 1H NMR (DMSO) δ: 11.73 (s, 1H), 10.94 (s, 1H), 8.05 (d, J=9.4 Hz, 1H), 6.60 (d, J=9.4 Hz, 1H), 3.81 (s, 3H).

Step 2: 4M HCl in dioxane (1.45 mL, 5.8 mmol) is added to a suspension of methyl 2,6-dihydroxy-3-nitrobenzoate (500 mg, 2.3 mmol) in triethyl ortho acetate (13.5 mL, 72 mmol) and the RM is evacuated/purged with N2 (3×) before 10% Pd/C (173 mg, 7 mol %) is added. The RM is evacuated/purged with H₂ (3×) and stirred under a H₂ atm for 16 h. The RM is filtered through a pad of celite and the filtrate concentrated in vacuo to give methyl 6-hydroxy-2-methylbenzo[d]oxazole-7-carboxylate as a yellow solid. LC-MS B: t_{R}=0.78 min; [M+H]⁺=208.32. ¹H NMR (DMSO) δ: 10.69 (s, 1H), 7.76 (d, J=8.7 Hz, 1H), 6.96 (d, J=8.7 Hz, 1H), 3.97 (s, 3H), 2.60 (s, 3H).

Steps 3-4: The title compound is prepared from methyl 6-hydroxy-2-methylbenzo[d]oxazole-7-carboxylate following the sequence of reactions described for B1, step 2 & step 3. LC-MS B: t_{R}=0.69 min; [M+H]⁺=341.35. Note: Title compound is unstable and should not be stored for prolonged periods.

### Methyl (R)-6-(2-amino-3-phenylpropoxy) imidazo[1,2-a]pyridine-5-carboxylate dihydrochloride (B3)

Step 1: Br2 (0.81 mL, 15.7 mmol) is added dropwise to a 0° C. soln. of methyl-3-hydroxypicolinate (2.41 g, 15.7 mmol) in water (110 mL) and the RM is warmed to RT and stirred overnight. The RM is quenched with 40% aq. sodium bisulfite soln. and extracted with DCM (2×). The combined org. extracts are washed with brine, dried over Na₂SO₄, filtered and evaporated in vacuo to give methyl 6-bromo-3-hydroxypicolinate as a white solid. LC-MS A: t_{R}=3.19 min; [M+H]⁺=231.9.

Step 2: Methyl (R)-6-bromo-3-(2-((tert-butoxycarbonyl)amino)-3-phenylpropoxy) picolinate is prepared from methyl 6-bromo-3-hydroxypicolinate and tert-butyl (R)-(1-hydroxy-3-phenylpropan-2-yl) carbamate in analogy to the procedure described for B-1.1 step 2. LC-MS C: t_{R}=2.16 min; [M+H−^{t}Bu]⁺=409.0.

Step 3: Pd₂(dba)₃ (483 mg, 0.53 mmol) and XPhos (201 mg, 0.42 mmol) are added to a RT mix. of methyl (R)-6-bromo-3-(2-((tert-butoxycarbonyl)amino)-3-phenylpropoxy) picolinate (5.0 g, 10.5 mmol), benzyl carbamate (1.67 g, 11.1 mmol) and Cs₂CO₃ (5.15 g, 15.8 mmol) in dioxane (130 mL) and the RM is heated to 95° C. and stirred for 48 h. The RM is cooled to RT, filtered and the filtrate is concentrated in vacuo before being purified by FC (eluting with 0% to 40% EtOAc in hept) to give methyl (R)-6-(((benzyloxy)carbonyl)amino)-3-(2-((tert-butoxycarbonyl)amino)-3-phenylpropoxy) picolinate as an orange solid. LC-MS C: t_{R}=2.24 min; [M+H]⁺=536.2.

Step 4: A soln. of methyl (R)-6-(((benzyloxy)carbonyl)amino)-3-(2-((tert-butoxycarbonyl)amino)-3-phenylpropoxy) picolinate (1.43 g, 2.19 mmol) in EtOH (20 mL) is purged with N2/vacuum (3×) before 10% Pd/C (70 mg, 0.07 mmol) is added. After inertizing another three times, a H₂ balloon is connected and the RM is stirred at 55° C. for 1 h. The mix. is filtered over a celite plug rinsing with EtOH. The filtrate is concentrated to give methyl (R)-6-amino-3-(2-((tert-butoxycarbonyl)amino)-3-phenylpropoxy) picolinate as a yellow oil. LC-MS G: t_{R}=2.00 min; [M+H]⁺=402.2.

Step 5: 50% aq. 2-Chloroacetaldehyde (0.475 mL, 3.74 mmol) is added to a mix. of methyl (R)-6-amino-3-(2-((tert-butoxycarbonyl)amino)-3-phenylpropoxy) picolinate (0.50 g, 1.25 mmol) and NaHCO₃ (209 mg, 2.49 mmol) in EtOH (15 mL) and the RM is heated to 70° C. and stirred for 5 h. The RM is concentrated in vacuo and the residue is partitioned between water and EtOAc and extracted. The layers are separated, and the aq. phase is re-extracted with EtOAc (2×). The combined org. extracts are washed with brine, dried over Na₂SO₄, filtered and evaporated in vacuo. The crude product is purified by FC (eluting with 50% to 100% EtOAc in hept) to give methyl (R)-6-(2-((tert-butoxycarbonyl)amino)-3-phenylpropoxy) imidazo[1,2-a]pyridine-5-carboxylate as a white solid. LC-MS F: t_{R}=1.01 min; [M+H]⁺=426.52.

Step 6: The title compound is prepared in analogy to the procedure described for B1, step 3. LC-MS G: t_{R}=1.83 min; [M+H]⁺=326.2.

### Benzyl (R)-6-(2-amino-3-phenylpropoxy)-3-fluoroquinoline-5-carboxylate dihydrochloride (B4)

Step 1: A soln. of Br2 (0.17 mL, 3.37 mmol) in AcOH (8.0 mL) is added to a RT soln. of 3-fluoroquinolin-6-ol (0.50 g, 3.06 mmol) and NaOAc (0.30 g, 3.68 mmol) in AcOH (20 mL) and the RM is stirred for 30 min. The RM is concentrated to dryness, the residue is partitioned between sat. aq. NaHCO₃ and EtOAc. The layers are separated, and the aq. layer is re-extracted with EtOAc (2×). The combined org. extracts are washed with brine, dried (Na₂SO₄), filtered, and evaporated to give 5-bromo-3-fluoroquinolin-6-ol as a brown solid. LC-MS G: t_{R}=1.38 min; [M+H]⁺=239.9.

Step 2: A soln. of 5-bromo-3-fluoroquinolin-6-ol (0.74 g, 3.06 mmol) in THF (15 mL) is added dropwise to a RT suspension of NaH (0.17 g, 4.29 mmol) in THF (15 mL) and the resulting mix. is stirred for 15 min before methoxymethyl bromide (0.3 mL, 3.67 mmol) is added dropwise at 0° C. After stirring for 1.5 h at 0° C. the RM is quenched by the addition of H₂O and extracted with EtOAc. The org. layer is washed with NaHCO₃, brine, dried (Na₂SO₄), filtered, and evaporated. The crude product is purified by FC (eluting with 2% to 30% EtOAc in hept) to give 5-bromo-3-fluoro-6-(methoxymethoxy) quinoline as a colourless oil. LC-MS G: t_{R}=2.03 min; No ionisation.

Step 3: nBuLi (1.6 M in hex, 0.98 mL, 1.57 mmol) is added dropwise to a −78° C. soln. of 5-bromo-3-fluoro-6-(methoxymethoxy) quinoline (300 mg, 1.05 mmol) in THF (18 mL) and the RM is stirred for 30 min. The RM is quenched with freshly ground dry ice (1.0 g, 22.7 mmol) and then warmed to RT and stirred for 30 min. The RM is concentrated in vacuo and the intermediate lithium carboxylate is dissolved in DMF (4 mL), then KHCO₃ (31.5 mg, 0.315 mmol) and BnBr (0.15 mL, 1.26 mmol) is added, and the RM is heated to 60° C. for 10 min. The RM is cooled to RT and partitioned between sat. aq. NaHCO₃ and EtOAc. The layers are separated, and the aq. layer is re-extracted with EtOAc (2×). The combined org. extracts are washed with brine, dried (Na₂SO₄), filtered, and evaporated. The crude product is purified by prep. HPLC (basic) to give benzyl 3-fluoro-6-(methoxymethoxy) quinoline-5-carboxylate as a yellow oil. LC-MS G: t_{R}=2.08 min; [M+H]⁺=342.10.

Step 4: TFA (0.24 mL, 3.13 mmol) is added to a RT soln. of benzyl 3-fluoro-6-(methoxymethoxy) quinoline-5-carboxylate (107 mg, 0.31 mmol) in DCM (3 mL) and the resulting mix. is stirred for 2 h at RT. The RM is concentrated in vacuo, the residue dissolved in EtOAc, and extracted with aq. sat NaHCO₃ sol. The aq. layer is extracted with EtOAc and the combined org. layers are washed with brine, dried (NaSO₄), filtered, and concentrated to give benzyl 3-fluoro-6-hydroxyquinoline-5-carboxylate as a slightly brownish oil. LC-MS G: t_{R}=2.06 min; [M−H]⁻=298.1.

Step 5: DIAD (0.064 mL, 0.33 mmol) is added to a 0° C. mix. of benzyl 3-fluoro-6-hydroxyquinoline-5-carboxylate (93.8 mg, 0.31 mmol), tert-butyl (R)-(1-hydroxy-3-phenylpropan-2-yl) carbamate (82 mg, 0.33 mmol) and PPh₃ (86 mg, 0.33 mmol) in THF (2 mL), and the RM is stirred for 16 h at RT. The mix. is concentrated and the residue directly purified by FC (eluting with 20% to 60% EtOAc in hept) to give benzyl (R)-6-(2-((tert-butoxycarbonyl)amino)-3-phenylpropoxy)-3-fluoroquinoline-5-carboxylate as a colourless oil. LC-MS G: t_{R}=2.39 min; [M−Boc+H]⁺=531.2.

Step 6: 4M HCl in dioxane (0.44 mL, 1.77 mmol) is added to a soln. of benzyl (R)-6-(2-((tert-butoxycarbonyl)amino)-3-phenylpropoxy)-3-fluoroquinoline-5-carboxylate (94 mg, 0.18 mmol) in dioxane (3 mL) and the RM is stirred for 24 h at RT. The volatiles are removed in vacuo and the residue is triturated with Et₂O (3×) to give the title compound as a white solid. LC-MS G: t_{R}=2.10 min; [M+H]⁺=431.2

### Benzyl (R)-6-(2-amino-3-phenylpropoxy)-8-methylquinoline-5-carboxylate (B5)

Step 1: Meldrum's acid (6.04 g, 41.1 mmol) and triethyl orthoformate (6.06 mL, 35.7 mmol) are added to a RT soln. of 4-methoxy-2-methylaniline (5.0 g, 35.7 mmol) in EtOH (50 mL) and the RM is heated to 80° C. for 2 h. The RM is cooled to RT and the precipitate is collected by filtration washing with EtOH and dried under HV to give 5-(((4-methoxy-2-methylphenyl)amino)methylene)-2,2-dimethyl-1,3-dioxane-4,6-dione as a white solid. LC-MS B: t_{R}=0.90 min; [M+H]⁺=292.13.

Step 2: 5-(((4-Methoxy-2-methylphenyl)amino)methylene)-2,2-dimethyl-1,3-dioxane-4,6-dione (8.19 g, 28.1 mmol) is dissolved in Dowtherm A (50 mL) and heated to 250° C. for 5 min. The RM is cooled to RT and diluted with Et₂O and the precipitate is collected by filtration and washed with Et₂O before being dried under HV to give 6-methoxy-8-methylquinolin-4-ol as a brown solid. LC-MS B: t_{R}=0.58 min; [M+H]⁺=190.21.

Step 3: Phosphorous tribromide (2.16 mL, 22.7 mmol) is added to a RT soln. of 6-methoxy-8-methylquinolin-4-ol (3.91 g, 20.7 mmol) in DMF (75 mL) and the RM is heated to 45° C. for 1 h. The RM is cooled to RT, diluted with water and the pH is adjusted to 8 by the addition of sat. aq. NaHCO₃ soln. The precipitate is collected by filtration and dissolved in EtOAc, washed with brine, dried over Na₂SO₄, filtered and evaporated in vacuo. The crude product is purified by FC (eluting with 10% EtOAc in hept) to give 4-bromo-6-methoxy-8-methylquinoline as a white solid. LC-MS B: t_{R}=0.82 min; [M+H]⁺=254.03.

Step 4: nBuLi (1.6 M in hex, 35.7 mL, 57.1 mmol) is added dropwise to a −78° C. soln. of 4-bromo-6-methoxy-8-methylquinoline (7.2 g, 28.5 mmol) in THF and the RM is stirred for 30 min. The reaction is quenched with sat. aq. NH₄Cl soln. and extracted with EtOAc (3×). The combined org. extracts are washed with brine, dried over Na₂SO₄, filtered and evaporated in vacuo. The crude product is purified by FC (eluting with 20% EtOAc in hept) to give 6-methoxy-8-methylquinoline as a yellow oil. LC-MS B: t_{R}=0.49 min; [M+H]⁺=174.26.

Step 5: Br2 (1.45 mL, 28.2 mmol) is added to a RT soln. of 6-methoxy-8-methylquinoline (2.44 g, 14.1 mmol) and NaOAc (1.39 g, 16.9 mmol) in AcOH (18.3 mL) and the RM is stirred for 10 min. The RM is quenched with sat. aq. NaHSO₃ and extracted with EtOAc (2×). The combined org. extracts are dried (MgSO₄), filtered, and concentrated in vacuo. The residue is taken up in PhMe and concentrated in vacuo (2×) to give 5-bromo-6-methoxy-8-methylquinoline as a green solid. LC-MS B: t_{R}=0.74 min; [M+H]⁺=252.09.

Step 6: BBr₃ (1 M in DCM, 42.5 mL, 42.5 mmol) is added dropwise to a 0° C. soln. of 5-bromo-6-methoxy-8-methylquinoline (3.57 g, 14.2 mmol) in DCM (70 mL). The cooling bath is removed and the RM is stirred at RT for 2 h. The RM is carefully quenched into cold MeOH and concentrated in vacuo. The residue is co-evaporated with PhMe, EtOAc and DCM to give 5-bromo-8-methylquinolin-6-ol as a yellow solid. LC-MS B: t_{R}=0.55 min; [M+H]⁺=238.01.

Step 7: tert-Butyl (R)-(1-((5-bromo-8-methylquinolin-6-yl)oxy)-3-phenylpropan-2-yl) carbamate is prepared from 5-bromo-8-methylquinolin-6-ol and tert-butyl (R)-(1-hydroxy-3-phenylpropan-2-yl) carbamate in analogy to the procedure described for B1 Step 2. LC-MS B: t_{R}=1.10 min; [M+H]⁺=472.94.

Step 8: nBuLi (1.6 M in hex, 0.54 mL, 0.86 mmol) is added dropwise to a −78° C. soln. of tert-butyl (R)-(1-((5-bromo-8-methylquinolin-6-yl)oxy)-3-phenylpropan-2-yl) carbamate (185 mg, 0.39 mmol) in THF (2 mL) and the RM is stirred for 30 min before benzyl chloroformate (0.058 mL, 0.41 mmol) is added dropwise. The RM is warmed to RT and quenched by addition of sat. aq. NaHCO₃ and extracted with EtOAc. The layers are separated and the aq. phase is re-extracted with EtOAc (2×) and the combined org. layers are washed with brine, dried over Na₂SO₄, filtered and evaporated in vacuo. The crude product is purified by prep. HPLC (acidic) to give benzyl (R)-6-(2-((tert-butoxycarbonyl)amino)-3-phenylpropoxy)-8-methylquinoline-5-carboxylate as a white solid. LC-MS B: t_{R}=1.11 min; [M+H]⁺=527.33.

Step 9: TFA (4.0 mL, 52.2 mmol) is added to a RT soln. of benzyl (R)-6-(2-((tert-butoxycarbonyl)amino)-3-phenylpropoxy)-8-methylquinoline-5-carboxylate (550 mg, 1.04 mmol) in DCM (5 mL) and the RM is stirred for 1 h. The RM is concentrated in vacuo and the residue is co-evaporated with DCM (2×) before being purified by prep. HPLC (basic) to give the title compound as a yellow oil. LC-MS B: t_{R}=0.78 min; [M+H]⁺=427.23.

### Methyl (R)-6-(2-amino-3-phenylpropoxy)-2-methylbenzofuran-7-carboxylate hydrochloride (B6)

Step 1: DMAP (120 mg, 0.99 mmol) is added to a 0° C. soln. of 2,6-dihydroxybenzoic acid (3.0 g, 19.7 mmol) in 1,2-dimethoxyethane (15 mL) followed by the dropwise addition of acetone (1.9 mL, 25.8 mmol) and thionyl chloride (1.85 mL, 25.2 mmol) and the RM is stirred for 30 min before being warmed to RT and stirred for 16 h. The RM is quenched by the addition of sat. aq. NaHCO₃ and extracted with Et₂O (4×). The combined org. extracts are washed with brine, dried over Na₂SO₄, filtered, and evaporated. The crude product is purified by FC (eluting with 0% to 50% EtOAc in hept) to give 5-hydroxy-2,2-dimethyl-4H-benzo[d][1,3]dioxin-4-one as a white solid. LC-MS C: t_{R}=1.93 min; [M+H]⁺=195.1.

Step 2: K₂CO₃ (2.38 g, 17.2 mmol) and 3-bromopropyne (80% soln. in PhMe, 1.67 mL, 15.5 mmol) are added to a RT soln. of 5-hydroxy-2,2-dimethyl-4H-benzo[d][1,3]dioxin-4-one (3.0 g, 15.4 mmol) in acetone (60 mL) and the RM is heated to 55° C. for 21 h. The mix. is concentrated, and the residue partitioned between water and EtOAc. The layers are separated and the aq. layer re-extracted with EtOAc (2×). The combined org. extracts are washed with brine, dried over Na₂SO₄, filtered, and evaporated in vacuo. The crude product is purified by FC (eluting with 5% to 35% EtOAc in hept) to give 2,2-dimethyl-5-(prop-2-yn-1-yloxy)-4H-benzo[d][1,3]dioxin-4-one as a white solid. LC-MS G: t_{R}=1.83 min; [M+H]⁺=233.1.

Step 3: NaOMe (30% soln. in MeOH, 1.9 mL, 10.1 mmol) is added to a 0° C. soln. of 2,2-dimethyl-5-(prop-2-yn-1-yloxy)-4H-benzo[d][1,3]dioxin-4-one (1.53 g, 6.6 mmol) in DMF (15 mL) and the RM is warmed to RT and stirred for 1 h. The RM is quenched with 1M aq. HCl and extracted with EtOAc (3×). The combined org. extracts are washed with brine, dried over Na₂SO₄, filtered, and evaporated in vacuo to give methyl 2-hydroxy-6-(prop-2-yn-1-yloxy)benzoate as a beige solid. LC-MS G: t_{R}=1.79 min; [M+H]⁺=207.0.

Step 4: A mix. of methyl 2-hydroxy-6-(prop-2-yn-1-yloxy)benzoate (1.33 g, 6.5 mmol), CsF (1.5 g, 9.9 mmol), and diethylaniline (18 mL) is purged with N₂ before being irradiated in a MW oven at 200° C. for 55 min. The RM is diluted with EtOAc and washed with 1M aq. HCl. The aq. phase is extracted with EtOAc (2×) and the combined org. extracts are washed with 1M HCl, brine, dried over Na₂SO₄, filtered, and evaporated in vacuo. The crude product is purified by FC (eluting with 1% to 15% EtOAc in hept) to give methyl 6-hydroxy-2-methylbenzofuran-7-carboxylate as a white solid. LC-MS C: t_{R}=1.98 min; [M+H]⁺=207.0.

Steps 5-6: The title compound is prepared from methyl 6-hydroxy-2-methylbenzofuran-7-carboxylate in analogy to the procedure described for B1 steps 2 & 3. LC-MS G: t_{R}=1.98 min; [M+H]⁺=340.1.

### Benzyl (R)-6-(2-amino-3-phenylpropoxy)-3-methylisoquinoline-5-carboxylate dihydrochloride (B7)

Step 1: Trifluoromethanesulfonic anhydride (26.2 mL, 158 mmol) is added dropwise to a −10° C. soln. of 2-hydroxy-4-methoxybenzaldehyde (16 g, 105 mmol) and pyridine (42.5 mL, 526 mmol) in DCM (70 mL) and the RM is stirred for 30 min. The RM is quenched with ice water and acidified with 1M aq. HCl before being extracted with EtOAc (2×). The combined org. extracts are washed with brine, dried over Na₂SO₄, filtered and evaporated in vacuo to give 2-formyl-5-methoxyphenyl trifluoromethanesulfonate as a yellow oil. ¹H NMR (400 MHZ, CDCl₃) δ 10.13 (s, 1H), 7.95 (d, J=8.8 Hz, 1H), 7.03 (dd, J=8.7, 2.3 Hz, 1H), 6.88 (d, J=2.3 Hz, 1H), 3.93 (s, 3H).

Step 2: A RT soln. of 2-formyl-5-methoxyphenyl trifluoromethanesulfonate (19.6 g, 66.4 mmol) and TEA (93 mL, 664 mmol) in DMF (400 mL) is purged with Ar for 30 min. Prop-1-yne (1 M in DMF, 133 mL, 133 mmol), CuI (1.27 g, 6.64 mmol) and Pd(PPh₃)₄ (5.0 g, 4.33 mmol) are added successively and the RM is stirred closed for 2 h. The RM is filtered through a pad of celite and the filtrate partially concentrated in vacuo before being diluted with EtOAc and washed successively with 1M KHSO₄ soln. and brine and concentrated in vacuo. The crude product is purified by FC (eluting with 0% to 30% EtOAc in hept) to give 4-methoxy-2-(prop-1-yn-1-yl)benzaldehyde as a yellow solid. LC-MS G: t_{R}=1.80 min; [M+H]⁺=175.1.

Step 3: A RT soln. of 4-methoxy-2-(prop-1-yn-1-yl)benzaldehyde (10.3 g, 58.8 mmol) in MeOH (350 mL) is purged with Ar for 5 min in an autoclave. NH₃ 7M in MeOH (150 mL, 1050 mmol) is added and the RM is heated to 65° C. for 4 h with a pressure reaching 2 bar. The RM is concentrated in vacuo and the residue is co-evaporated with DCM (2×) to give 6-methoxy-3-methylisoquinoline as a brown solid. LC-MS G: t_{R}=1.81 min; [M+H]⁺=174.1.

Step 4: BBr₃ (1 M in DCM, 55.4 mL, 55.4 mmol) is added dropwise to a −78° C. soln. of 6-methoxy-3-methylisoquinoline (5.0 g, 27.7 mmol) in DCM (100 mL). The cooling bath is removed and the RM is stirred at RT for 30 h. The RM is carefully quenched into cold MeOH and concentrated in vacuo. The residue is co-evaporated with PhMe, EtOAc and DCM to give 3-methylisoquinolin-6-ol as a brown solid. LC-MS G: t_{R}=1.10 min; [M+H]⁺=160.1.

Step 5: Br2 (1.3 mL, 25.3 mmol) is added dropwise to a suspension of 3-methylisoquinolin-6-ol (4.67 g, 19.4 mmol) in CHCl₃ (75 mL) and the RM is stirred for 2 h. EtOAc is added and the solids are collected by filtration, washed with EtOAc and hept. The filter residue is neutralised by suspending it in sat. aq. NaHCO₃ and re-filtered before washing with H₂O and hept. The filter residue is suspended in MeCN and evaporated to give 5-bromo-3-methylisoquinolin-6-ol as a brown solid. LC-MS G: t_{R}=1.02 min; [M+H]⁺=238.0.

Step 6: tert-Butyl (R)-(1-((5-bromo-3-methylisoquinolin-6-yl)oxy)-3-phenylpropan-2-yl) carbamate is prepared from 5-bromo-3-methylisoquinolin-6-ol and tert-butyl (R)-(1-hydroxy-3-phenylpropan-2-yl) carbamate in analogy to the procedure described for B1 Step 2. LC-MS G: t_{R}=2.21 min; [M+H]⁺=471.1.

Step 7: A RT soln. of tert-butyl (R)-(1-((5-bromo-3-methylisoquinolin-6-yl)oxy)-3-phenylpropan-2-yl) carbamate (2.5 g, 5.30 mmol), benzyl alcohol (2.76 mL, 26.5 mmol) and DIPEA (2.78 mL, 15.9 mmol) in PhMe (20 mL) is purged with Ar for 10 min. The RM is then purged with CO and heated to 88° C. under a CO atm before a soln. of Pd(^{t}Bu₃P)₂ (271 mg, 0.53 mmol) in PhMe (5.5 mL) is added via syringe pump (3 mL/h). The temperature is increased to 95° C. and the RM is stirred under a CO atm for 24 h. The RM is cooled to RT and concentrated in vacuo and the residue is partitioned between sat. aq. NaHCO₃ and EtOAc and extracted. The layers are separated and the aq. phase is re-extracted with EtOAc (1×) and the combined org. layers are washed with brine, dried over Na₂SO₄, filtered and evaporated in vacuo. The crude product is purified by FC (eluting with 5% to 65% EtOAc in hept) to give benzyl (R)-6-(2-((tert-butoxycarbonyl)amino)-3-phenylpropoxy)-3-methylisoquinoline-5-carboxylate as a colourless oil. LC-MS G: t_{R}=2.19 min; [M+H]⁺=527.2.

Step 8: The title compound is prepared from benzyl (R)-6-(2-((tert-butoxycarbonyl)amino)-3-phenylpropoxy)-3-methylisoquinoline-5-carboxylate in analogy to the procedure described for B1, step 3. LC-MS G: t_{R}=1.95 min; [M+H]⁺=427.2.

### Benzyl (R)-3-(2-amino-3-phenylpropoxy)-6-fluoroquinoline-4-carboxylate dihydrochloride (B8)

The title compound is prepared from 6-fluoro-3-hydroxyquinoline-4-carboxylic acid in analogy to the procedure described for B1, steps 1-3. LC-MS F: t_{R}=1.12 min; [M+H]⁺=431.24.

### Preparation of Building Blocks C

### N—(N-(tert-Butoxycarbonyl)-N-methyl-L-leucyl)-N-methyl-D-alanine (C1)

Step 1: Mel (1.0 mL, 16.06 mmol) is added to a 0° C. soln. of (tert-butoxycarbonyl)-D-alanine (2.01 g, 10.62 mmol) in THF (10 mL), then NaH (1.08 g, 27.1 mmol) is added. After 30 min at 0° C., the RM is warmed to RT and stirring is continued for 3 h. The mix. is quenched with H₂O and acidified with 0.5 M KHSO₄ (pH 2). The layers are separated and the aq. layer is extracted with EtOAc (3×20 mL). The combined org. layers are washed with brine, dried (Na₂SO₄), filtered, and evaporated to yield N-(tert-butoxycarbonyl)-N-methyl-D-alanine as a brown oil which is used as such in the next step.

Step 2: K₂CO₃ (2.84 g, 20.5 mmol) is added to a RT soln. of N-(tert-butoxycarbonyl)-N-methyl-D-alanine (2.16 g, 10.6 mmol) in acetone (6 mL) followed by the dropwise addition of BnBr (1.4 mL, 11.8 mmol). The resulting mix. is heated to 50° C. and stirred for 2.5 h. The mix. is cooled to RT, filtered and the filtrate concentrated to give benzyl N-(tert-butoxycarbonyl)-N-methyl-D-alaninate which was used as such in the next step. LC-MS G: t_{R}=2.19 min; [M+H]⁺=No ionization.

Step 3: 4 M HCl in dioxane (0.56 mL, 12.0 mmol) is added to a RT soln. of benzyl N-(tert-butoxycarbonyl)-N-methyl-D-alaninate (11.0 g, 34.2 mmol) in DCM (3 mL) and the resulting mix. is stirred for 18 h. The suspension is filtered and washed with Et₂O (2×) to give benzyl methyl-D-alaninate HCl as a white solid. LC-MS G: t_{R}=1.80 min; [M+H]⁺=194.2.

Step 4: A soln. of benzyl methyl-D-alaninate HCl (0.82 g, 3.6 mmol) and DIPEA (1.0 mL, 5.75 mmol) in DMF (1 mL) is added to a pre-stirred RT soln. of Boc-N-methyl-L-leucine (0.90 mg, 3.7 mmol), HATU (1.39 g, 3.7 mmol) and DIPEA (1.0 mL, 5.75 mmol) in DMF (6 mL) and the resulting mix. is stirred for 18 h. The mix. is concentrated, and the residue partitioned between H₂O and EtOAc. The layers are separated and the aq. layer re-extracted with EtOAc (2×). The combined org. extracts are washed with brine, dried (MgSO₄), filtered, and evaporated to give the crude product that is triturated with Et₂O to give benzyl N—(N-(tert-butoxycarbonyl)-N-methyl-L-leucyl)-N-methyl-D-alaninate as a colourless oil. LC-MS F: t_{R}=2.33 min; [M+H]⁺=421.2.

Step 5: A soln. of benzyl N—(N-(tert-butoxycarbonyl)-N-methyl-L-leucyl)-N-methyl-D-alaninate (1.32 g, 3.13 mmol) in EtOH (20 mL) is purged with N2/vacuum (3×) before 10% Pd/C (333 mg, 10 mol %) is added. The RM is evacuated/purged with H₂ (3×) and stirred under a H₂ atm for 2 h. The mix. is concentrated and filtered over a celite plug, rinsing with EtOH. The filtrate is concentrated to give the title compound as a colourless oil. LC-MS C: t_{R}=1.98 min; [M+H]⁺=331.20. ¹H NMR (400 MHZ, DMSO) § 5.03-4.46 (m, 2H), 2.95-2.83 (m, 2H), 2.75-2.54 (m, 4H), 1.58-1.44 (m, 2H), 1.41 (s, 10H), 1.31-1.23 (m, 3H), 1.23-1.17 (m, 1H), 0.94-0.84 (m, 6H).

### 2-(S)-2-((tert-Butoxycarbonyl)(methyl)amino)-N,4-dimethylpentanamido)-4,4,4-trifluorobutanoic acid (C2)

Step 1: NaOAc (12.78 g, 0.156 mol), TFA (2.41 mL, 31.1 mmol) and benzaldehyde (3.34 mL, 32.7 mmol) are added to a RT solution of methyl 2-amino-4,4,4-trifluorobutanoate hydrochloride (6.81 g, 31.1 mmol) in MeOH (20 mL) and the resulting mix. is stirred at RT for 1 h. NaBH₃CN (2.27 g, 34.3 mmol) is then added and stirring is continued for 45 min. The mixture is evaporated to dryness, then partitioned between H₂O and DCM, and the layers are separated. The aq. layer is extracted with DCM and the combined org. extracts are dried (Na₂SO₄), filtered, and evaporated to give rac-methyl (R)-2-(benzylamino)-4,4,4-trifluorobutanoate as a brown oil, which is used as such in the next step. LC-MS F: t_{R}=0.96 min; [M+H]⁺=262.37.

Step 2: NaOAc (12.67 g, 155 mmol), TFA (2.39 mL, 30.9 mmol) and formaldehyde (37% in H₂O, 2.53 mL, 34 mmol) are added to a RT solution of rac-methyl (R)-2-(benzylamino)-4,4,4-trifluorobutanoate (8.07 g, 30.9 mmol) in MeOH (100 mL) and the resulting mix. is stirred at RT for 1 h. NaBH₃CN (2.25 g, 34.0 mmol) is then added and stirring is continued. After 1.5 h, formaldehyde (37% in H₂O, 0.46 mL, 6.18 mmol) and NaBH₃CN (409 mg, 6.18 mmol) are added and the mix. is stirred for another 2 h at RT. The mix. is evaporated to dryness, partitioned between H₂O and DCM, and the layers separated. The aq. layer is re-extracted with DCM and the combined org. extracts are dried (Na₂SO₄), filtered, and evaporated to give rac-methyl (R)-2-(benzyl(methyl)amino)-4,4,4-trifluorobutanoate as a brown oil, which is used as such in the next step. LC-MS F: t_{R}=0.96 min; [M+H]⁺=262.37.

Step 3: A solution of rac-methyl (R)-2-(benzyl(methyl)amino)-4,4,4-trifluorobutanoate (6.48 g, 23.5 mmol) in EtOH (200 mL) is evacuated/purged with Ar (3×) before Pd/C (1.25 g, 5 mol %) is added. The RM is evacuated/purged with H₂ (3×) and stirred under a H₂ atm for 2.5 h. The mix. is filtered and the solid is rinsed with MeOH. 4M HCl (5.89 mL, 23.5 mmol) is added and the mix. is evaporated to dryness to give rac-methyl (R)-4,4,4-trifluoro-2-(methylamino) butanoate hydrochloride as an off-white solid which is used as such in the next step. LC-MS F: t_{R}=0.6 min; [M+H]⁺=186.37.

Step 4: HATU (11.26 g, 29.6 mmol) is added portion wise to a RT soln. of Boc-N-methyl-L-leucine (6.24 g, 24.7 mmol), rac-methyl (R)-4,4,4-trifluoro-2-(methylamino) butanoate hydrochloride (5.47 g, 24.7 mmol), and DIPEA (16.9 mL, 98.7 mmol) in DMF (80 mL) and the resulting mix. is stirred for 1 h. Water is added and the mix. is extracted with EtOAc (3×). The combined org. extracts are successively washed with sat. aq. NaHCO₃, H₂O, and brine, dried (Na₂SO₄), filtered, and concentrated. Purification by FC (eluting with 15% EtOAc in hept) gives methyl-2-((S)-2-((tert-butoxycarbonyl)(methyl)amino)-N,4-dimethylpentanamido)-4,4,4-trifluorobutanoate as a yellowish oil. LC-MS B: t_{R}=1.06 min; [M+H]⁺=413.29.

Step 5: 2 M aq. NaOH (6.9 mL, 13.8 mmol) is added to a RT soln. of methyl-2-((S)-2-((tert-butoxycarbonyl)(methyl)amino)-N,4-dimethylpentanamido)-4,4,4-trifluorobutanoate (2.84 g, 6.88 mmol) in MeOH (10 mL) and the mix. is stirred at RT for 1.5 h. The volatiles are removed in vacuo and the aq. residue is neutralised with 2 M aq. HCl before being extracted with DCM (3×). The combined org. layers are dried (Na₂SO₄), filtered, and evaporated to give the title compound as a white solid. LC-MS B: t_{R}=0.86 min; [M+H]⁺=359.49. LC-MS B: t_{R}=0.96 min; [M+H]⁺=399.27.

### (R)-2-((S)-2-(((Allyloxy)carbonyl)(methyl)amino)-N,4-dimethylpentanamido)-2-cyclopentylacetic acid (C3)

Step 1:4 M HCl in dioxane (11 mL, 44 mmol) is added to a RT soln. of Boc-N-Me-L-leucine (2.78 g, 11 mmol) in DCM (20 mL). The mix. is stirred at RT for 2 h. The RM is concentrated to yield(S)-4-methyl-2-methylamino-pentanoic acid (2.14 g) which is used as such in the next step.

Step 2: Allyl chloroformate (1.27 mL, 11.6 mmol) is added to(S)-4-methyl-2-methylamino-pentanoic acid (1.59 g, 11 mmol) and Na₂CO₃ (4.08 g, 38.5 mmol) in dioxane/H₂O 3/5 (48 mL). The mix. is stirred at RT overnight, then the RM is diluted with EtOAc and acidified to pH 2 using 2 M aq. HCl. The layers are separated and the aq. layer is extracted with EtOAc (2×). The combined org. layers are dried (Na₂SO₄), filtered, and concentrated to yield N-(allyloxy)carbonyl)-N-methyl-L-leucine (2.5 g, 99%) which is used as such in the next step. LC-MS B: t_{R}=0.80 min; [M+H]⁺=230.43.

Step 3: NaH (60% dispersion in mineral oil, 265 mg, 6.92 mmol) is added to a 0° C. soln. of methyl (R)-2-((tert-butoxycarbonyl)amino)-2-cyclopentylacetate (420 mg, 1.73 mmol) in DMF (8 mL), then Mel (0.87 mL, 13.8 mmol) is added and the RM is warmed to RT overnight. The solvent is evaporated and the crude product is purified by FC to yield methyl (R)-2-((tert-butoxycarbonyl)(methyl)amino)-2-cyclopentylacetate. LC-MS B: t_{R}=1.03 min; [M+H]⁺=272.28.

Step 4: 4 M HCl in dioxane (0.32 mL, 1.29 mmol) is added to a 0° C. soln. of methyl (R)-2-((tert-butoxycarbonyl)(methyl)amino)-2-cyclopentylacetate (350 mg, 1.29 mmol) in DCM (3 mL) and the mix. is stirred at RT for 2 h. N-((Allyloxy)carbonyl)-N-methyl-L-leucine (from step 2, 296 mg, 1.29 mmol), DIPEA (0.68 mL, 3.87 mmol), and PyCloP (666 mg, 1.55 mmol) are added to the RM at RT, then the mix. is heated to 40° C. for 2 h. Water is added to the RM and the product is extracted with DCM (2×). The combined org. layers are dried (MgSO₄), filtered, and concentrated. by yields Purification FC methyl (R)-2-((S)-2-(((allyloxy)carbonyl)(methyl)amino)-N,4-dimethylpentanamido)-2-cyclopentylacetate. LC-MS B: t_{R}=1.08 min; [M+H]⁺=383.24.

Step 5: LiOH·H₂O (37.5 mg, 0.89 mmol) is added to a RT soln. of methyl (R)-2-((S)-2-(((allyloxy)carbonyl)(methyl)amino)-N,4-dimethylpentanamido)-2-cyclopentylacetate (171 mg, 0.45 mmol) in a solvent mix. of MeOH/H₂O 3/1 (2.5 mL) and the RM is heated to 100° C. for 15 h. MeOH is evaporated and the residue is acidified with 2 M HCl to pH 1, then extracted with EtOAc (3×). The combined org. layers are dried (MgSO₄), filtered, and evaporated to yield the title compound, which is used as such in the next step. LC-MS B: t_{R}=0.96 min; [M+H]⁺=369.13.

### (R)-2-((S)-2-((tert-Butoxycarbonyl)(methyl)amino)-N,4-dimethylpentanamido)-4,4-difluorobutanoic acid (C4)

Step 1: 1M aq. NaOH (13.5 mL, 13.5 mmol) is added to a RT suspension of (R)-2-amino-4,4-difluorobutanoic acid (1.0 g, 7.19 mmol) in dioxane (20 mL). Boc₂O (1.64 g, 7.42 mmol) is added to the RM and stirred at RT for 24 h. The mixture is concentrated and the residue is acidified with 4 M aq. potassium hydrogensulfate and extracted with DCM. The combined org. extracts are dried (MgSO₄), filtered, and concentrated. The crude residue is re-dissolved in MeCN and washed with hept then coevaporated with Et₂O to give (R)-2-((tert-butoxycarbonyl)amino)-4,4-difluorobutanoic acid as a colourless oil, which is used as such in the next step. LC-MS B: t_{R}=0.70 min; [M+H]⁺=240.26

Step 2: NaH 60% dispersion in mineral oil (0.46 g, 12.1 mmol) is added to a 0° C. suspension of (R)-2-(tert-butoxycarbonyl)amino)-4,4-difluorobutanoic acid (1.41 g, 5.89 mmol) in DMF (23 mL). The RM is stirred at 0° C. for 10 min, then at RT for another 10 min. The RM is cooled back to 0° C., then Mel (0.76 mL, 12.1 mmol) is added dropwise and the RM is left returning to RT overnight. Water and EtOAc are added, then the two layers are separated and the aq. layer is extracted further with EtOAc (2×). The combined org. layers are dried (Na₂SO₄), filtered, and concentrated to yield methyl (R)-2-((tert-butoxycarbonyl)(methyl)amino)-4,4-difluorobutanoate as a yellow oil, which is used as such in the next step. LC-MS B: t_{R}=0.88 min; [M+H]⁺=268.17.

Step 3: TFA (4.51 mL, 58.9 mmol) is added to a RT soln. of methyl (R)-2-((tert-butoxycarbonyl)(methyl)amino)-4,4-difluorobutanoate (1.57 g, 5.89 mmol) in DCM (40 mL) and the RM is stirred at RT for 2 h. The volatiles are removed in vacuo, and the residue co-evaporated with DCM (3×) to give methyl (R)-4,4-difluoro-2-(methylamino) butanoate 2,2,2-trifluoroacetate which is used as such in the next step. LC-MS B: t_{R}=0.24 min; [M+H]⁺=168.05.

Steps 4 and 5: The title compound is prepared from methyl (R)-4,4-difluoro-2-(methylamino) butanoate 2,2,2-trifluoroacetate and Boc-N-methyl-L-leucine, following the sequence of reactions described for C2, steps 4&5. LC-MS B: t_{R}=0.93 min; [M+H]⁺=381.26.

Listed in Table C below are building blocks C that are prepared from Boc-N-methyl-L-leucine and the corresponding SM in analogy to the 4-step sequence described above for C4 (step 2-5). Alternatively, in step 3, Boc deprotection can be performed in the presence of 4 M HCl in dioxane instead of TFA.

TABLE C

| | | | | | MS- |
|---|---|---|---|---|---|
| | | | | t_{R} [min] | data |
| | | | | LC-MS | m/z |
| No. | SM Step 1 | SM Step 3 | Product | Method | [M + H]⁺ |
| C5 | Boc-D-Val-OH | Boc-N-methyl- | N-(N-(tert-Butoxycarbonyl)-N- | 0.96 | 359.33 |
| | | L-leucine | methyl-L-leucyl)-N-methyl-D-valine | B | |
| C6 | Boc-D-Leu-OH | Boc-N-methyl- | N-(N-(tert-Butoxycarbonyl)-N- | 1.00 | 373.33 |
| | | L-leucine | methyl-L-leucyl)-N-methyl-D-leucine | B | |
| C7 | (R)-N-Boc-2- | Boc-N-Methyl- | (R)-2-((S)-2-((tert- | 0.92 | 345.31 |
| | aminobutyric acid⁺ | L-leucine | Butoxycarbonyl)(methyl)amino)-N,4- | B | |
| | | | dimethylpentanamido)butanoic acid | | |

### Synthesis of Compounds of Formula (I)

### (3S,7S,10R,13R)-13-benzyl-7-isobutyl-N-(2-(3-methoxy-1,2,4-oxadiazol-5-yl)ethyl)-6,9,20-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]isoquinoline-3-carboxamide (Example 1)

Step 1: HATU (829 mg, 2.12 mmol) is added to a RT soln. of B7 (1.50 g, 2.01 mmol), C2 (803 mg, 2.01 mmol) and DIPEA (1.38 mL, 8.06 mmol) in DMF (25 mL) and the RM is stirred for 2.5 h. The RM is partitioned between H₂O and EtOAc. The layers are separated and the aq. layer is re-extracted with EtOAc (2×). The combined org. layers are washed with sat. aq. NaHCO₃, 1M aq. citric acid, H₂O, and brine, dried (Na₂SO₄), filtered, and evaporated. The crude is purified by prep. HPLC (basic) to give benzyl 6-(((6S,9R,12R)-12-benzyl-6-isobutyl-2,2,5,8-tetramethyl-4,7,10-trioxo-9-(2,2,2-trifluoroethyl)-3-oxa-5,8,11-triazatridecan-13-yl)oxy)-3-methylisoquinoline-5-carboxylate (779 mg) with LC-MS F: t_{R}=1.40 min; [M+H]⁺=808.04 and benzyl 6-(((6S,9S,12R)-12-benzyl-6-isobutyl-2,2,5,8-tetramethyl-4,7,10-trioxo-9-(2,2,2-trifluoroethyl)-3-oxa-5,8,11-triazatridecan-13-yl)oxy)-3-methylisoquinoline-5-carboxylate (296 mg) with LC-MS F: t_{R}=1.37 min; [M+H]⁺=808.03.

Step 2: A soln. of benzyl 6-(((6S,9R,12R)-12-benzyl-6-isobutyl-2,2,5,8-tetramethyl-4,7,10-trioxo-9-(2,2,2-trifluoroethyl)-3-oxa-5,8,11-triazatridecan-13-yl)oxy)-3-methylisoquinoline-5-carboxylate (779 mg, 0.965 mmol) in EtOH (10 mL) is evacuated/purged with N₂ (3×) before 10% Pd/C (51.4 mg, 5 mol %) is added. The RM is evacuated/purged with H₂ (3×) and stirred under a H₂ atm for 2 h. The RM is filtered through a pad of celite and the filtrate concentrated to give 6-(((6S,9R,12R)-12-benzyl-6-isobutyl-2,2,5,8-tetramethyl-4,7,10-trioxo-9-(2,2,2-trifluoroethyl)-3-oxa-5,8,11-triazatridecan-13-yl)oxy)-3-methylisoquinoline-5-carboxylic acid as a white solid. LC-MS F: t_{R}=0.69 min; [M+H]⁺=717.90.

Step 3: HATU (27.9 mg, 0.07 mmol) is added to a RT soln. of 6-(((6S,9R,12R)-12-benzyl-6-isobutyl-2,2,5,8-tetramethyl-4,7,10-trioxo-9-(2,2,2-trifluoroethyl)-3-oxa-5,8,11-triazatridecan-13-yl)oxy)-3-methylisoquinoline-5-carboxylic acid (50 mg, 0.07 mmol), A7 (27.4 mg, 0.07 mmol) and DIPEA (0.037 uL, 0.21 mmol) in DMF (1.5 mL) and the RM is stirred for 10 min. The RM is directly purified by prep. HPLC (basic) to give tert-butyl(S)-3-(6-(((6S,9R,12R)-12-benzyl-6-isobutyl-2,2,5,8-tetramethyl-4,7,10-trioxo-9-(2,2,2-trifluoroethyl)-3-oxa-5,8,11-triazatridecan-13-yl)oxy)-3-methylisoquinoline-5-carboxamido)-4-((2-(3-methoxy-1,2,4-oxadiazol-5-yl)ethyl)amino)-4-oxobutanoate as a white solid. LC-MS F: t_{R}=1.28 min; [M+H]⁺=1014.33.

Step 4: TFA (0.40 mL, 5.0 mmol) is added to a RT soln. of tert-butyl(S)-3-(6-(((6S,9R,12R)-12-benzyl-6-isobutyl-2,2,5,8-tetramethyl-4,7,10-trioxo-9-(2,2,2-trifluoroethyl)-3-oxa-5,8,11-triazatridecan-13-yl)oxy)-3-methylisoquinoline-5-carboxamido)-4-((2-(3-methoxy-1,2,4-oxadiazol-5-yl)ethyl)amino)-4-oxobutanoate (32 mg, 0.03 mmol) in DCM (1.5 mL) and the RM is stirred for 1.5 h. The RM is concentrated and the residue is re-dissolved in DCM and again concentrated (2×). The residue is dissolved in DMF (1.5 mL) before DIPEA (0.04 mL, 0.25 mmol) and HATU (14.4 mg, 0.04 mmol) is added and the RM is stirred for 5 min. The RM is directly purified by prep. HPLC (basic) to give the title compound as a white solid. LC-MS E: t_{R}=0.84 min; [M+H]⁺=839.4.

Note: In cases where the product of step 1 above is a methyl or ethyl ester instead of the described benzyl ester (e.g. Example 41 below), a basic hydrolysis using 10 eq. 2M aq. NaOH soln. in MeOH at RT or heated up to 80° C. is performed. The subsequent reaction sequence then remains the same as described for Example 1. In some cases, chiral chromatography is used to obtain the desired product as a pure stereoisomer. In some cases, the product of step 1 is kept as a mixture of stereoisomers, and the synthesis sequence is performed on a mixture, resulting in a final example, being a mixture of epimers. In some cases, the mixtures of stereoisomers were separated by chiral chromatography.

Listed in Table MC-1 below are compounds of general formula (I) prepared from the corresponding building blocks A, B, and C in analogy to the synthesis described for Example 1.

TABLE MC-1

| | | | | t_{R} [min] | MS-data m/z |
|---|---|---|---|---|---|
| Ex. | A | B | C | LC-MS Method E | [M + H]⁺ |
| 2* | A7 | B7 | C2 | 0.82 | 839.4 |
| 3 | A7 | B4 | C2 | 1.13 | 843.3 |
| 4* | A7 | B4 | C2 | 1.11 | 843.3 |
| 5 | A1 | B1 | C5 | 1.19 | 768.6 |
| 6 | A1 | B1 | C6 | 1.25 | 782.6 |
| 7 | A1 | B1 | C1 | 1.09 | 740.7 |
| 8 | A5 | B1 | C1 | 1.15 | 766.7 |
| 9 | A1 | B1 | C4 | 1.15 | 790.7 |
| 10 | A6 | B4 | C1 | 1.11 | 774.4 |
| 11 | A9 | B4 | C1 | 1.15 | 784.5 |
| 12 | A6 | B3 | C1 | 0.80 | 745.5 |
| 13 | A5 | B3 | C1 | 0.83 | 755.5 |
| 14 | A5 | B4 | C1 | 1.16 | 784.5 |
| 15 | A2 | B4 | C1 | 1.17 | 784.5 |
| 16 | A3 | B4 | C1 | 1.13 | 785.5 |
| 17 | A4 | B4 | C1 | 1.15 | 785.4 |
| 18 | A3 | B4 | C4 | 1.20 | 835.5 |
| 19* | A3 | B4 | C4 | 1.17 | 835.5 |
| 20 | A3 | B4 | C7 | 1.18 | 799.4 |
| 21 | A6 | B4 | C4 | 1.17 | 824.4 |
| 22 | A1 | B7 | C1 | 0.80 | 754.5 |
| 23 | A6 | B7 | C1 | 0.81 | 770.5 |
| 24 | A4 | B7 | C1 | 0.83 | 781.5 |
| 25 | A8 | B4 | C4 | 1.12 | 835.4 |
| 26 | A6 | B7 | C4 | 0.82 | 820.4 |
| 27 | A7 | B7 | C4 | 0.80 | 821.4 |
| 28 | A8 | B7 | C4 | 0.84 | 831.4 |
| 29 | A1 | B4 | C4 | 1.10 | 808.4 |
| 30 | A7 | B4 | C4 | 1.08 | 825.4 |
| 31 | A3 | B7 | C2 | 0.87 | 849.4 |
| 32 | A4 | B7 | C2 | 0.88 | 849.4 |
| 33 | A5 | B7 | C2 | 0.89 | 848.4 |
| 34 | A6 | B7 | C2 | 0.86 | 838.4 |
| 35 | A2 | B7 | C2 | 0.90 | 848.4 |
| 36 | A8 | B7 | C2 | 0.87 | 849.4 |
| 37 | A10 | B7 | C2 | 0.90 | 856.4 |
| 38 | A10 | B4 | C4 | 1.15 | 842.3 |
| 39 | A10 | B7 | C4 | 0.86 | 838.4 |
| 40 | A7 | B5 | C2 | 1.08 | 839.4 |
| 41 | A6 | B2 | C2 | 1.09 | 828.4 |
| 42 | A6 | B2 | C2 | 1.16 | 828.8 |
| 43 | A1 | B6 | C1 | 1.12 | 743.7 |

*denotes an example compound isolated during the synthesis, most often separated by prep. HPLC purification of the final synthetic step as a minor epimer due to epimerisation of a chiral centre. In certain cases, an enantiomerically or diastereomerically pure building block(s) undergoes epimersation during the synthesis and the example compound is isolated as a mixture of epimers.

### (3S,7S,10R,13R)-13-benzyl-10-cyclopentyl-7-isobutyl-6,9-dimethyl-N-(2-(3-methylisoxazol-5-yl)ethyl)-1,5,8,11-tetraoxo-1,2,3,4,5,6,7,8,9,10,11,12,13,14-tetradecahydro-[1]oxa[4,7,10,14]tetraazacycloheptadecino[17,16-c]quinoline-3-carboxamide (Ex 44)

Step 1: T3P (50% in DMF, 0.50 mL, 0.82 mmol) is added to a RT soln. of C3 (152 mg, 0.41 mmol, B1 (200 mg, 0.41 mmol) and DIPEA (0.22 mL, 1.24 mmol) in DCM (1 mL) and the RM is stirred at elevated temperature for 2 h. H₂O is added and the mix. is extracted with DCM. The org. layer is concentrated and the crude residue purified by FC to give benzyl 3-(((2R,5R,8R)-2-benzyl-5-cyclopentyl-8-isobutyl-6,9-dimethyl-4,7,10-trioxo-11-oxa-3,6,9-triazatetradec-13-en-1-yl)oxy) quinoline-4-carboxylate.

Step 2: LiOH·H₂O (19.3 mg, 0.456 mmol) is added to a RT soln. of benzyl 3-(((2R,5R,8R)-2-benzyl-5-cyclopentyl-8-isobutyl-6,9-dimethyl-4,7,10-trioxo-11-oxa-3,6,9-triazatetradec-13-en-1-yl)oxy) quinoline-4-carboxylate (174 mg, 0.23 mmol) in a solvent mix of THF/H₂O 2/1 (1.0 mL) and the RM is heated to 50° C. for 3 d. THF is evaporated and the residue is acidified with 2 M HCl to pH 3, then extracted with DCM (3×). The combined org. layers are dried (MgSO₄), filtered, and evaporated to give 3-(((2R,5R,8R)-2-benzyl-5-cyclopentyl-8-isobutyl-6,9-dimethyl-4,7,10-trioxo-11-oxa-3,6,9-triazatetradec-13-en-1-yl)oxy) quinoline-4-carboxylic acid, which is used as such in the next step. LC-MS B: t_{R}=1.09 min; [M+H]⁺=673.09.

Step 3: HATU (95.1 mg, 0.25 mmol) is added to a RT soln. of 3-(((2R,5R,8R)-2-benzyl-5-cyclopentyl-8-isobutyl-6,9-dimethyl-4,7,10-trioxo-11-oxa-3,6,9-triazatetradec-13-en-1-yl)oxy) quinoline-4-carboxylic acid (150 mg, 0.22 mmol), A11 (70.8 mg, 0.0.22 mmol), and DIPEA (0.153 mL, 0.989 mmol) in DCM (2 mL) and the RM is stirred at RT for 3 h. Purification by FC gives allyl (S)-3-(3-(((2R,5R,8S)-2-benzyl-5-cyclopentyl-8-isobutyl-6,9-dimethyl-4,7,10-trioxo-11-oxa-3,6,9-triazatetradec-13-en-1-yl)oxy) quinoline-4-carboxamido)-4-((2-(3-methylisoxazol-5-yl)ethyl)amino)-4-oxobutanoate.

Step 4: Pd(Ph₃)₄ (20.2 mg, 0.017 mmol) is added to a RT soln. of allyl (S)-3-(3-(((2R,5R,8S)-2-benzyl-5-cyclopentyl-8-isobutyl-6,9-dimethyl-4,7,10-trioxo-11-oxa-3,6,9-triazatetradec-13-en-1-yl)oxy) quinoline-4-carboxamido)-4-((2-(3-methylisoxazol-5-yl)ethyl)amino)-4-oxobutanoate (160 mg, 0.171 mmol) and 1,3-dimethylbarbituric acid (53.9 mg, 0.342 mmol) in DCM (2 mL) and the RM is stirred at RT for 1 h. The RM is concentrated to give(S)-3-(3-((R)-2-((R)-2-cyclopentyl-2-((S)—N,4-dimethyl-2-(methylamino) pentanamido) acetamido)-3-phenylpropoxy) quinoline-4-carboxamido)-4-((2-(3-methylisoxazol-5-yl)ethyl)amino)-4-oxobutanoic acid which is used as such in the next step.

Step 5: HATU (66.3 mg, 0.171 mmol) is added to a RT soln. of(S)-3-(3-((R)-2-((R)-2-cyclopentyl-2-((S)—N,4-dimethyl-2-(methylamino) pentanamido) acetamido)-3-phenylpropoxy) quinoline-4-carboxamido)-4-((2-(3-methylisoxazol-5-yl)ethyl)amino)-4-oxobutanoic acid (max 0.171 mmol) and DIPEA (90 UL, 0.51 mmol) in DCM (2 mL) and the RM is stirred at RT for 30 min. Purification by prep. HPLC (basic) gives the title compound. LC-MS E: t_{R}=1.27 min; [M+H]⁺=794.6.

Listed in the Table MC-2 below is a compound of general formula (I) prepared from the corresponding building blocks A, B, and C in analogy to the synthesis described for Example 44.

TABLE MC-2

| | | | | t_{R} [min] | MS-data m/z |
|---|---|---|---|---|---|
| Ex. | A | B | C | LC-MS Method E | [M + H]⁺ |
| 45 | A11 | B8 | C3 | 1.31 | 812.6 |

Listed in the Table of Examples below are example compounds of formula (I) prepared according to the above described methods. The configuration at stereocenters that are not mentioned in the compound name are unknown however only one epimer is present.

Table of Examples

| Ex. | Compound of Formula (I) |
|---|---|
| 1 | (3S,7S,10R,13R)-13-benzyl-7-isobutyl-N-(2-(3-methoxy-1,2,4-oxadiazol-5-yl)ethyl)-6,9,20- |
| | trimethyl-1,5,8,11-tetraoxo-10-(2,2,2-trifluoroethyl)-1,2,3,4,5,6,7,8,9,10,11,12,13,14- |
| | tetradecahydro-[1]oxa[4,7,10,14]tetraazacycloheptadecino[16,17-f]isoquinoline-3-carboxamide |
| 2 | (3S,7S,10S,13R)-13-benzyl-7-isobutyl-N-(2-(3-methoxy-1,2,4-oxadiazol-5-yl)ethyl)-6,9,20- |
| | trimethyl-1,5,8,11-tetraoxo-10-(2,2,2-trifluoroethyl)-1,2,3,4,5,6,7,8,9,10,11,12,13,14- |
| | tetradecahydro-[1]oxa[4,7,10,14]tetraazacycloheptadecino[16,17-f]isoquinoline-3-carboxamide |
| 3 | (3S,7S,10R,13R)-13-benzyl-20-fluoro-7-isobutyl-N-(2-(3-methoxy-1,2,4-oxadiazol-5-yl)ethyl)-6,9- |
| | dimethyl-1,5,8,11-tetraoxo-10-(2,2,2-trifluoroethyl)-1,2,3,4,5,6,7,8,9,10,11,12,13,14- |
| | tetradecahydro-[1]oxa[4,7,10,14]tetraazacycloheptadecino[16,17-f]quinoline-3-carboxamide |
| 4 | (3S,7S,10S,13R)-13-benzyl-20-fluoro-7-isobutyl-N-(2-(3-methoxy-1,2,4-oxadiazol-5-yl)ethyl)-6,9- |
| | dimethyl-1,5,8,11-tetraoxo-10-(2,2,2-trifluoroethyl)-1,2,3,4,5,6,7,8,9,10,11,12,13,14- |
| | tetradecahydro-[1]oxa[4,7,10,14]tetraazacycloheptadecino[16,17-f]quinoline-3-carboxamide |
| 5 | (3S,7S,10R,13R)-13-benzyl-7-isobutyl-10-isopropyl-6,9-dimethyl-N-(2-(3-methylisoxazol-5- |
| | yl)ethyl)-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 6 | (3S,7S,10R,13R)-13-benzyl-7,10-diisobutyl-6,9-dimethyl-N-(2-(3-methylisoxazol-5-yl)ethyl)- |
| | 1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 7 | (3S,7S,10R,13R)-13-benzyl-7-isobutyl-6,9,10-trimethyl-N-(2-(3-methylisoxazol-5-yl)ethyl)-1,5,8,11- |
| | tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 8 | (3S,7S,10R,13R)-13-benzyl-N-(2-(3-cyclopropylisoxazol-5-yl)ethyl)-7-isobutyl-6,9,10-trimethyl- |
| | 1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 9 | (3S,7S,10R,13R)-13-benzyl-10-(2,2-difluoroethyl)-7-isobutyl-6,9-dimethyl-N-(2-(3-methylisoxazol- |
| | 5-yl)ethyl)-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 10 | (3S,7S,10R,13R)-13-benzyl-20-fluoro-7-isobutyl-N-(2-(3-methoxyisoxazol-5-yl)ethyl)-6,9,10- |
| | trimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 11 | (3S,7S,10R,13R)-13-benzyl-N-(2-(4-cyclopropyl-2H-1,2,3-triazol-2-yl)ethyl)-20-fluoro-7-isobutyl- |
| | 6,9,10-trimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 12 | (3S,7S,10R,13R)-13-benzyl-7-isobutyl-N-(2-(3-methoxyisoxazol-5-yl)ethyl)-6,9,10-trimethyl- |
| | 1,5,8,11-tetraoxo-1,2,3,4,5,6,7,8,9,10,11,12,13,14-tetradecahydroimidazo[1′,2′:1,6]pyrido[2,3- |
| | p][1]oxa[4,7,10,14]tetraazacycloheptadecine-3-carboxamide |
| 13 | (3S,7S,10R,13R)-13-benzyl-N-(2-(3-cyclopropylisoxazol-5-yl)ethyl)-7-isobutyl-6,9,10-trimethyl- |
| | 1,5,8,11-tetraoxo-1,2,3,4,5,6,7,8,9,10,11,12,13,14-tetradecahydroimidazo[1′,2′:1,6]pyrido[2,3- |
| | p][1]oxa[4,7,10,14]tetraazacycloheptadecine-3-carboxamide |
| 14 | (3S,7S,10R,13R)-13-benzyl-N-(2-(3-cyclopropylisoxazol-5-yl)ethyl)-20-fluoro-7-isobutyl-6,9,10- |
| | trimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 15 | (3S,7S,10R,13R)-13-benzyl-N-(2-(5-cyclopropylisoxazol-3-yl)ethyl)-20-fluoro-7-isobutyl-6,9,10- |
| | trimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 16 | (3S,7S,10R,13R)-13-benzyl-N-(2-(5-cyclopropyl-1,2,4-oxadiazol-3-yl)ethyl)-20-fluoro-7-isobutyl- |
| | 6,9,10-trimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 17 | (3S,7S,10R,13R)-13-benzyl-N-(2-(3-cyclopropyl-1,2,4-oxadiazol-5-yl)ethyl)-20-fluoro-7-isobutyl- |
| | 6,9,10-trimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 18 | (3S,7S,10R,13R)-13-benzyl-N-(2-(5-cyclopropyl-1,2,4-oxadiazol-3-yl)ethyl)-10-(2,2-difluoroethyl)- |
| | 20-fluoro-7-isobutyl-6,9-dimethyl-1,5,8,11-tetraoxo-1,2,3,4,5,6,7,8,9,10,11,12,13,14- |
| | tetradecahydro-[1]oxa[4,7,10,14]tetraazacycloheptadecino[16,17-f]quinoline-3-carboxamide |
| 19 | (3S,7S,10S,13R)-13-benzyl-N-(2-(5-cyclopropyl-1,2,4-oxadiazol-3-yl)ethyl)-10-(2,2-difluoroethyl)- |
| | 20-fluoro-7-isobutyl-6,9-dimethyl-1,5,8,11-tetraoxo-1,2,3,4,5,6,7,8,9,10,11,12,13,14- |
| | tetradecahydro-[1]oxa[4,7,10,14]tetraazacycloheptadecino[16,17-f]quinoline-3-carboxamide |
| 20 | (3S,7S,10R,13R)-13-benzyl-N-(2-(5-cyclopropyl-1,2,4-oxadiazol-3-yl)ethyl)-10-ethyl-20-fluoro-7- |
| | isobutyl-6,9-dimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 21 | (3S,7S,10R,13R)-13-benzyl-10-(2,2-difluoroethyl)-20-fluoro-7-isobutyl-N-(2-(3-methoxyisoxazol-5- |
| | yl)ethyl)-6,9-dimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 22 | (3S,7S,10R,13R)-13-benzyl-7-isobutyl-6,9,10,20-tetramethyl-N-(2-(3-methylisoxazol-5-yl)ethyl)- |
| | 1,5,8,11-tetraoxo-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 23 | (3S,7S,10R,13R)-13-benzyl-7-isobutyl-N-(2-(3-methoxyisoxazol-5-yl)ethyl)-6,9,10,20-tetramethyl- |
| | 1,5,8,11-tetraoxo-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 24 | (3S,7S,10R,13R)-13-benzyl-N-(2-(3-cyclopropyl-1,2,4-oxadiazol-5-yl)ethyl)-7-isobutyl-6,9,10,20- |
| | tetramethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 25 | (3S,7S,10R,13R)-13-benzyl-N-(2-(5-cyclopropyl-2H-tetrazol-2-yl)ethyl)-10-(2,2-difluoroethyl)-20- |
| | fluoro-7-isobutyl-6,9-dimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 26 | (3S,7S,10R,13R)-13-benzyl-10-(2,2-difluoroethyl)-7-isobutyl-N-(2-(3-methoxyisoxazol-5-yl)ethyl)- |
| | 6,9,20-trimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 27 | (3S,7S,10R,13R)-13-benzyl-10-(2,2-difluoroethyl)-7-isobutyl-N-(2-(3-methoxy-1,2,4-oxadiazol-5- |
| | yl)ethyl)-6,9,20-trimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 28 | (3S,7S,10R,13R)-13-benzyl-N-(2-(5-cyclopropyl-2H-tetrazol-2-yl)ethyl)-10-(2,2-difluoroethyl)-7- |
| | isobutyl-6,9,20-trimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 29 | (3S,7S,10R,13R)-13-benzyl-10-(2,2-difluoroethyl)-20-fluoro-7-isobutyl-6,9-dimethyl-N-(2-(3- |
| | methylisoxazol-5-yl)ethyl)-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 30 | (3S,7S,10R,13R)-13-benzyl-10-(2,2-difluoroethyl)-20-fluoro-7-isobutyl-N-(2-(3-methoxy-1,2,4- |
| | oxadiazol-5-yl)ethyl)-6,9-dimethyl-1,5,8,11-tetraoxo-1,2,3,4,5,6,7,8,9,10,11,12,13,14- |
| | tetradecahydro-[1]oxa[4,7,10,14]tetraazacycloheptadecino[16,17-f]quinoline-3-carboxamide |
| 31 | (3S,7S,10R,13R)-13-benzyl-N-(2-(5-cyclopropyl-1,2,4-oxadiazol-3-yl)ethyl)-7-isobutyl-6,9,20- |
| | trimethyl-1,5,8,11-tetraoxo-10-(2,2,2-trifluoroethyl)-1,2,3,4,5,6,7,8,9,10,11,12,13,14- |
| | tetradecahydro-[1]oxa[4,7,10,14]tetraazacycloheptadecino[16,17-f]isoquinoline-3-carboxamide |
| 32 | (3S,7S,10R,13R)-13-benzyl-N-(2-(3-cyclopropyl-1,2,4-oxadiazol-5-yl)ethyl)-7-isobutyl-6,9,20- |
| | trimethyl-1,5,8,11-tetraoxo-10-(2,2,2-trifluoroethyl)-1,2,3,4,5,6,7,8,9,10,11,12,13,14- |
| | tetradecahydro-[1]oxa[4,7,10,14]tetraazacycloheptadecino[16,17-f]isoquinoline-3-carboxamide |
| 33 | (3S,7S,10R,13R)-13-benzyl-N-(2-(3-cyclopropylisoxazol-5-yl)ethyl)-7-isobutyl-6,9,20-trimethyl- |
| | 1,5,8,11-tetraoxo-10-(2,2,2-trifluoroethyl)-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 34 | (3S,7S,10R,13R)-13-benzyl-7-isobutyl-N-(2-(3-methoxyisoxazol-5-yl)ethyl)-6,9,20-trimethyl- |
| | 1,5,8,11-tetraoxo-10-(2,2,2-trifluoroethyl)-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 35 | (3S,7S,10R,13R)-13-benzyl-N-(2-(5-cyclopropylisoxazol-3-yl)ethyl)-7-isobutyl-6,9,20-trimethyl- |
| | 1,5,8,11-tetraoxo-10-(2,2,2-trifluoroethyl)-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 36 | (3S,7S,10R,13R)-13-benzyl-N-(2-(5-cyclopropyl-2H-tetrazol-2-yl)ethyl)-7-isobutyl-6,9,20-trimethyl- |
| | 1,5,8,11-tetraoxo-10-(2,2,2-trifluoroethyl)-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 37 | (3S,7S,10R,13R)-13-benzyl-N-(2-(4-fluoro-3-methoxyisoxazol-5-yl)ethyl)-7-isobutyl-6,9,20- |
| | trimethyl-1,5,8,11-tetraoxo-10-(2,2,2-trifluoroethyl)-1,2,3,4,5,6,7,8,9,10,11,12,13,14- |
| | tetradecahydro-[1]oxa[4,7,10,14]tetraazacycloheptadecino[16,17-f]isoquinoline-3-carboxamide |
| 38 | (3S,7S,10R,13R)-13-benzyl-10-(2,2-difluoroethyl)-20-fluoro-N-(2-(4-fluoro-3-methoxyisoxazol-5- |
| | yl)ethyl)-7-isobutyl-6,9-dimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]quinoline-3-carboxamide |
| 39 | (3S,7S,10R,13R)-13-benzyl-10-(2,2-difluoroethyl)-N-(2-(4-fluoro-3-methoxyisoxazol-5-yl)ethyl)-7- |
| | isobutyl-6,9,20-trimethyl-1,5,8,11-tetraoxo-1,2,3,4,5,6,7,8,9,10,11,12,13,14-tetradecahydro- |
| | [1]oxa[4,7,10,14]tetraazacycloheptadecino[16,17-f]isoquinoline-3-carboxamide |
| 40 | (3S,7S,10R,13R)-13-benzyl-7-isobutyl-N-(2-(3-methoxy-1,2,4-oxadiazol-5-yl)ethyl)-6,9,17- |
| | trimethyl-1,5,8,11-tetraoxo-10-(2,2,2-trifluoroethyl)-1,2,3,4,5,6,7,8,9,10,11,12,13,14- |
| | tetradecahydro-[1]oxa[4,7,10,14]tetraazacycloheptadecino[16,17-f]quinoline-3-carboxamide |
| 41 | (8R,11RS,14S,18S)-8-benzyl-14-isobutyl-N-(2-(3-methoxyisoxazol-5-yl)ethyl)-2,12,15-trimethyl- |
| | 10,13,16,20-tetraoxo-11-(2,2,2-trifluoroethyl)-7,8,9,10,11,12,13,14,15,16,17,18,19,20- |
| | tetradecahydrooxazolo[4′,5′:5,6]benzo[1,2-p][1]oxa[4,7,10,14]tetraazacycloheptadecine-18- |
| | carboxamide |
| 42 | (8R,11R,14S,18S)-8-benzyl-14-isobutyl-N-(2-(3-methoxyisoxazol-5-yl)ethyl)-2,12,15-trimethyl- |
| | 10,13,16,20-tetraoxo-11-(2,2,2-trifluoroethyl)-7,8,9,10,11,12,13,14,15,16,17,18,19,20- |
| | tetradecahydrooxazolo[4′,5′:5,6]benzo[1,2-p][1]oxa[4,7,10,14]tetraazacycloheptadecine-18- |
| | carboxamide |
| 43 | (8R,11RS,14S,18S)-8-benzyl-14-isobutyl-2,11,12,15-tetramethyl-N-(2-(3-methylisoxazol-5- |
| | yl)ethyl)-10,13,16,20-tetraoxo-7,8,9,10,11,12,13,14,15,16,17,18,19,20- |
| | tetradecahydrobenzofuro[7,6-p][1]oxa[4,7,10,14]tetraazacycloheptadecine-18-carboxamide |
| 44 | (3S,7S,10R,13R)-13-benzyl-10-cyclopentyl-7-isobutyl-6,9-dimethyl-N-(2-(3-methylisoxazol-5- |
| | yl)ethyl)-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |
| 45 | (3S,7S,10R,13R)-13-benzyl-10-cyclopentyl-20-fluoro-7-isobutyl-6,9-dimethyl-N-(2-(3- |
| | methylisoxazol-5-yl)ethyl)-1,5,8,11-tetraoxo-1,2,3,4,5,6,7,8,9,10,11,12,13,14-tetradecahydro- |
| | [1]oxa[4,7,10,14]tetraazacycloheptadecino[17,16-c]quinoline-3-carboxamide |

TABLE S
Structures of compounds of Example 1 to 45

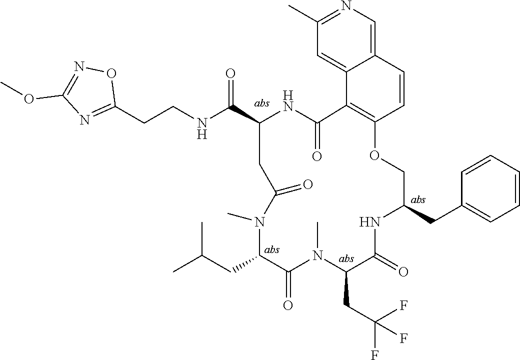 [SMILES: COc1noc(CCNC(=O)[C@@H]2CC(=O)N(C)[C@@H](CC(C)C)C(=O)N(C)[C@H](CC(F)(F)F)C(=O)N[C@H](Cc3ccccc3)COc3ccc4cnc(C)cc4c3C(=O)N2)n1]

1

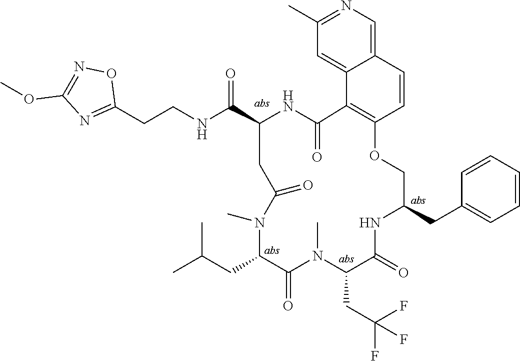 [SMILES: COc1noc(CCNC(=O)[C@@H]2CC(=O)N(C)[C@@H](CC(C)C)C(=O)N(C)[C@@H](CC(F)(F)F)C(=O)N[C@H](Cc3ccccc3)COc3ccc4cnc(C)cc4c3C(=O)N2)n1]

2

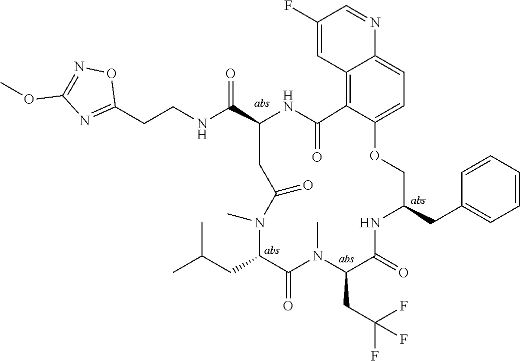 [SMILES: COc1noc(CCNC(=O)[C@@H]2CC(=O)N(C)[C@@H](CC(C)C)C(=O)N(C)[C@H](CC(F)(F)F)C(=O)N[C@H](Cc3ccccc3)COc3ccc4ncc(F)cc4c3C(=O)N2)n1]

3

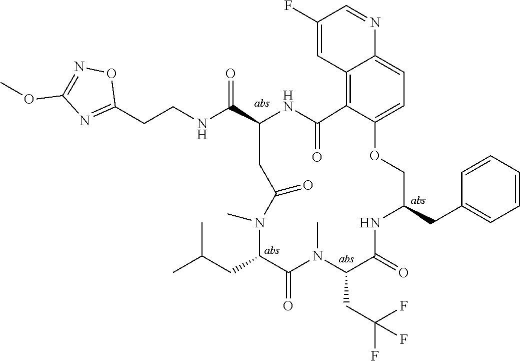 [SMILES: COc1noc(CCNC(=O)[C@@H]2CC(=O)N(C)[C@@H](CC(C)C)C(=O)N(C)[C@@H](CC(F)(F)F)C(=O)N[C@H](Cc3ccccc3)COc3ccc4ncc(F)cc4c3C(=O)N2)n1]

4

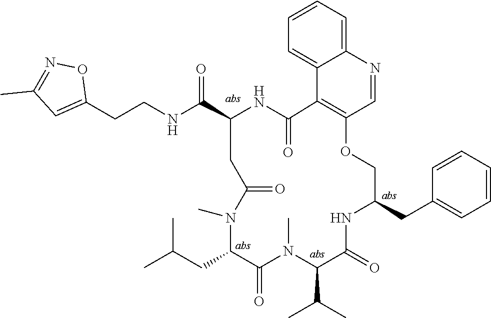 [SMILES: Cc1cc(CCNC(=O)[C@@H]2CC(=O)N(C)[C@@H](CC(C)C)C(=O)N(C)[C@H](C(C)C)C(=O)N[C@H](Cc3ccccc3)COc3cnc4ccccc4c3C(=O)N2)on1]

5

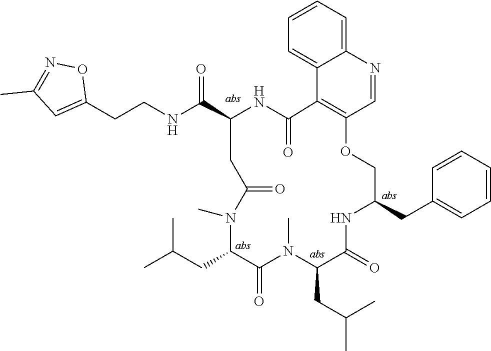 [SMILES: Cc1cc(CCNC(=O)[C@@H]2CC(=O)N(C)[C@@H](CC(C)C)C(=O)N(C)[C@H](CC(C)C)C(=O)N[C@H](Cc3ccccc3)COc3cnc4ccccc4c3C(=O)N2)on1]

6

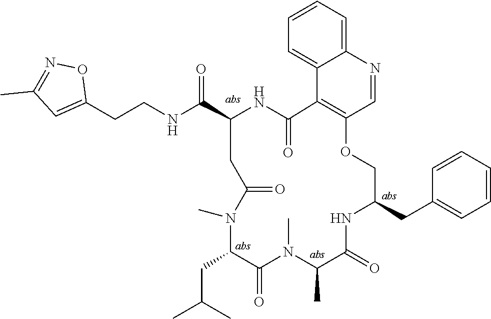 [SMILES: Cc1cc(CCNC(=O)[C@@H]2CC(=O)N(C)[C@@H](CC(C)C)C(=O)N(C)[C@H](C)C(=O)N[C@H](Cc3ccccc3)COc3cnc4ccccc4c3C(=O)N2)on1]

7

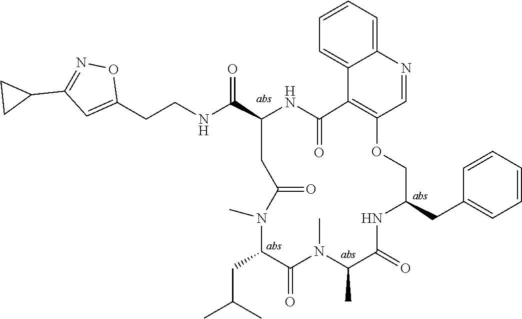 [SMILES: CC(C)C[C@H]1C(=O)N(C)[C@H](C)C(=O)N[C@H](Cc2ccccc2)COc2cnc3ccccc3c2C(=O)N[C@H](C(=O)NCCc2cc(C3CC3)no2)CC(=O)N1C]

8

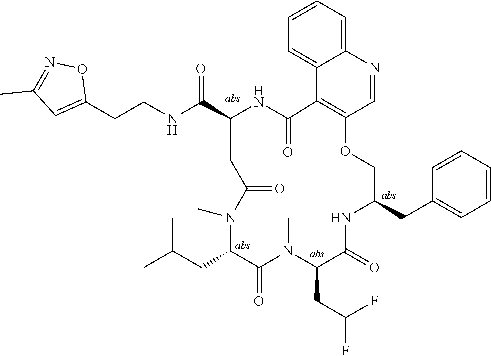 [SMILES: Cc1cc(CCNC(=O)[C@@H]2CC(=O)N(C)[C@@H](CC(C)C)C(=O)N(C)[C@H](CC(F)F)C(=O)N[C@H](Cc3ccccc3)COc3cnc4ccccc4c3C(=O)N2)on1]

9

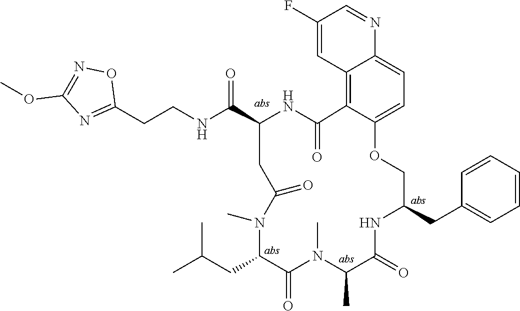 [SMILES: COc1noc(CCNC(=O)[C@@H]2CC(=O)N(C)[C@@H](CC(C)C)C(=O)N(C)[C@H](C)C(=O)N[C@H](Cc3ccccc3)COc3ccc4ncc(F)cc4c3C(=O)N2)n1]

10

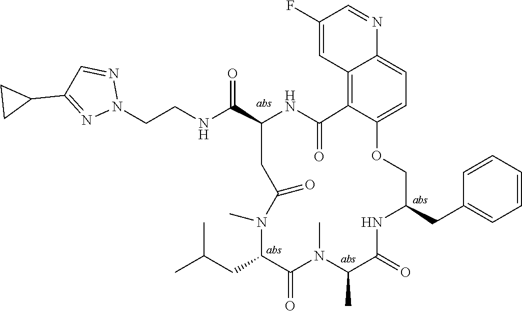 [SMILES: CC(C)C[C@H]1C(=O)N(C)[C@H](C)C(=O)N[C@H](Cc2ccccc2)COc2ccc3ncc(F)cc3c2C(=O)N[C@H](C(=O)NCCn2ncc(C3CC3)n2)CC(=O)N1C]

11

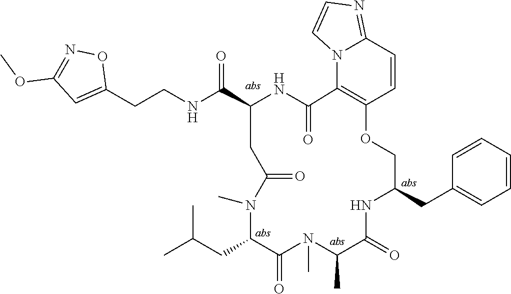 [SMILES: COc1cc(CCNC(=O)[C@@H]2CC(=O)N(C)[C@@H](CC(C)C)C(=O)N(C)[C@H](C)C(=O)N[C@H](Cc3ccccc3)COc3ccc4nccn4c3C(=O)N2)on1]

12

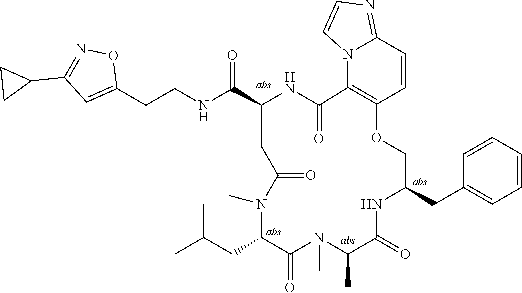 [SMILES: CC(C)C[C@H]1C(=O)N(C)[C@H](C)C(=O)N[C@H](Cc2ccccc2)COc2ccc3nccn3c2C(=O)N[C@H](C(=O)NCCc2cc(C3CC3)no2)CC(=O)N1C]

13

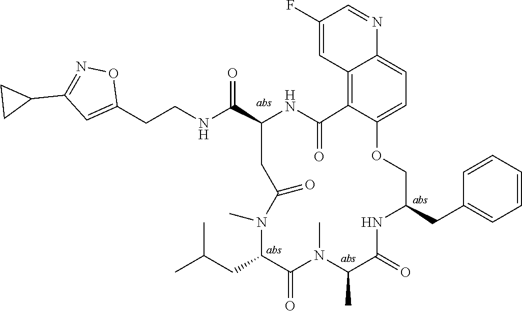 [SMILES: CC(C)C[C@H]1C(=O)N(C)[C@H](C)C(=O)N[C@H](Cc2ccccc2)COc2ccc3ncc(F)cc3c2C(=O)N[C@H](C(=O)NCCc2cc(C3CC3)no2)CC(=O)N1C]

14

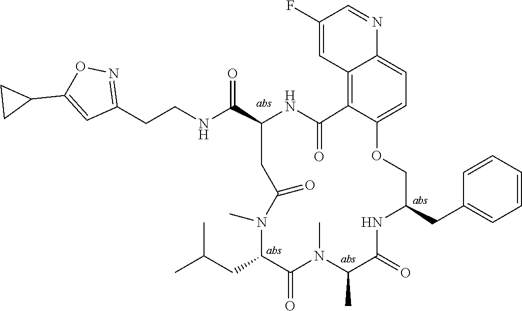 [SMILES: CC(C)C[C@H]1C(=O)N(C)[C@H](C)C(=O)N[C@H](Cc2ccccc2)COc2ccc3ncc(F)cc3c2C(=O)N[C@H](C(=O)NCCc2cc(C3CC3)on2)CC(=O)N1C]

15

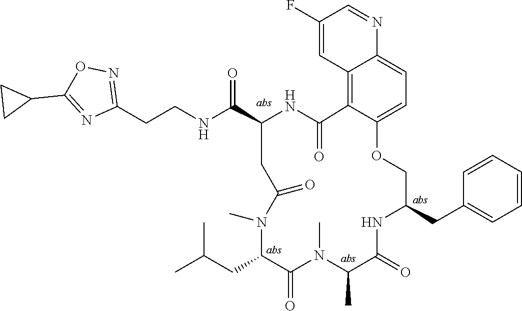 [SMILES: CC(C)C[C@H]1C(=O)N(C)[C@H](C)C(=O)N[C@H](Cc2ccccc2)COc2ccc3ncc(F)cc3c2C(=O)N[C@H](C(=O)NCCc2noc(C3CC3)n2)CC(=O)N1C]

16

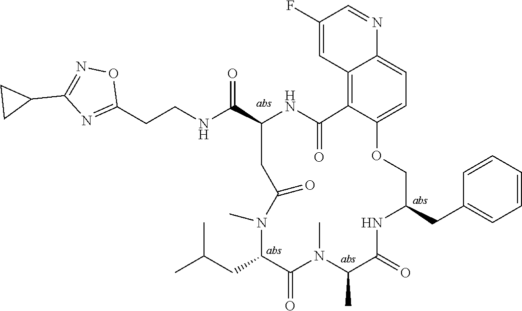 [SMILES: CC(C)C[C@H]1C(=O)N(C)[C@H](C)C(=O)N[C@H](Cc2ccccc2)COc2ccc3ncc(F)cc3c2C(=O)N[C@H](C(=O)NCCc2nc(C3CC3)no2)CC(=O)N1C]

17

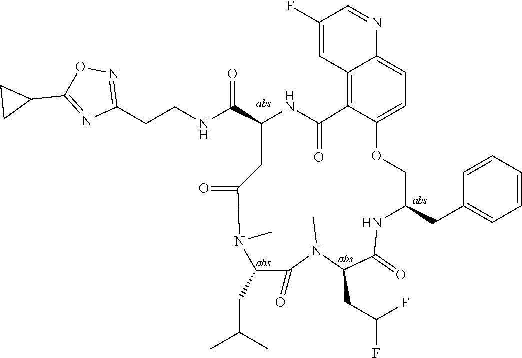 [SMILES: CC(C)C[C@H]1C(=O)N(C)[C@H](CC(F)F)C(=O)N[C@H](Cc2ccccc2)COc2ccc3ncc(F)cc3c2C(=O)N[C@H](C(=O)NCCc2noc(C3CC3)n2)CC(=O)N1C]

18

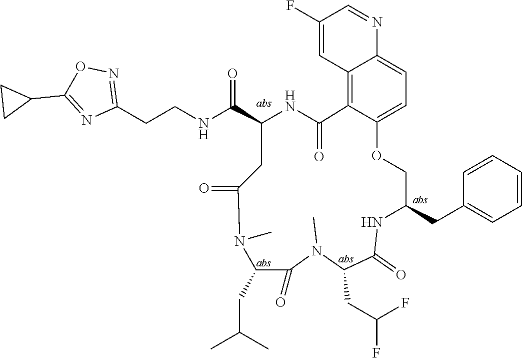 [SMILES: CC(C)C[C@H]1C(=O)N(C)[C@@H](CC(F)F)C(=O)N[C@H](Cc2ccccc2)COc2ccc3ncc(F)cc3c2C(=O)N[C@H](C(=O)NCCc2noc(C3CC3)n2)CC(=O)N1C]

19

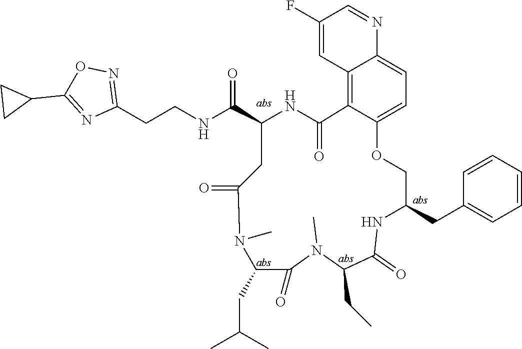 [SMILES: CC[C@@H]1C(=O)N[C@H](Cc2ccccc2)COc2ccc3ncc(F)cc3c2C(=O)N[C@H](C(=O)NCCc2noc(C3CC3)n2)CC(=O)N(C)[C@@H](CC(C)C)C(=O)N1C]

20

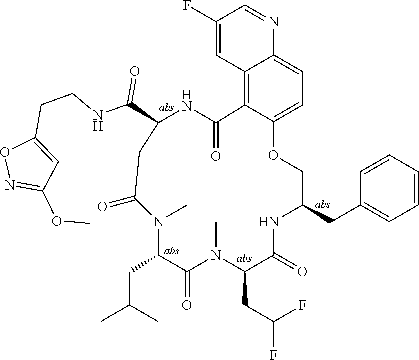 [SMILES: COc1cc(CCNC(=O)[C@@H]2CC(=O)N(C)[C@@H](CC(C)C)C(=O)N(C)[C@H](CC(F)F)C(=O)N[C@H](Cc3ccccc3)COc3ccc4ncc(F)cc4c3C(=O)N2)on1]

21

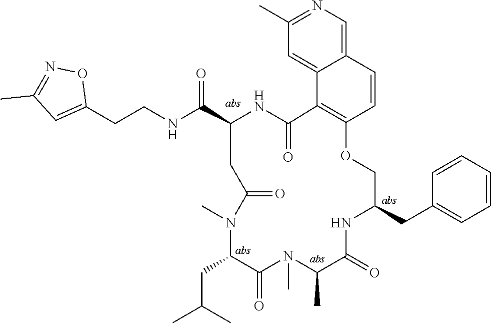 [SMILES: Cc1cc2c3c(ccc2cn1)OC[C@@H](Cc1ccccc1)NC(=O)[C@@H](C)N(C)C(=O)[C@H](CC(C)C)N(C)C(=O)C[C@@H](C(=O)NCCc1cc(C)no1)NC3=O]

22

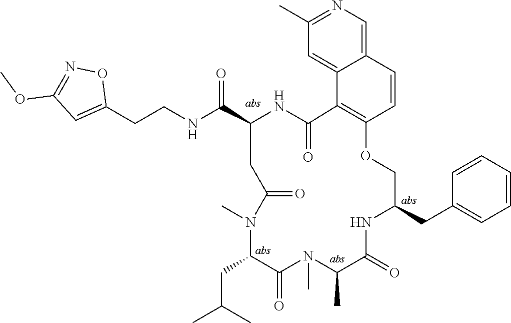 [SMILES: COc1cc(CCNC(=O)[C@@H]2CC(=O)N(C)[C@@H](CC(C)C)C(=O)N(C)[C@H](C)C(=O)N[C@H](Cc3ccccc3)COc3ccc4cnc(C)cc4c3C(=O)N2)on1]

23

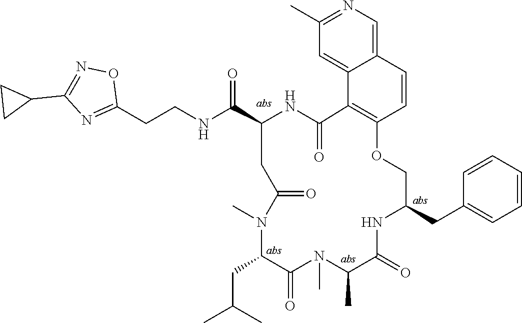 [SMILES: Cc1cc2c3c(ccc2cn1)OC[C@@H](Cc1ccccc1)NC(=O)[C@@H](C)N(C)C(=O)[C@H](CC(C)C)N(C)C(=O)C[C@@H](C(=O)NCCc1nc(C2CC2)no1)NC3=O]

24

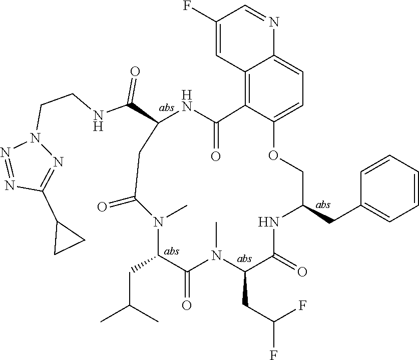 [SMILES: CC(C)C[C@H]1C(=O)N(C)[C@H](CC(F)F)C(=O)N[C@H](Cc2ccccc2)COc2ccc3ncc(F)cc3c2C(=O)N[C@H](C(=O)NCCn2nnc(C3CC3)n2)CC(=O)N1C]

25

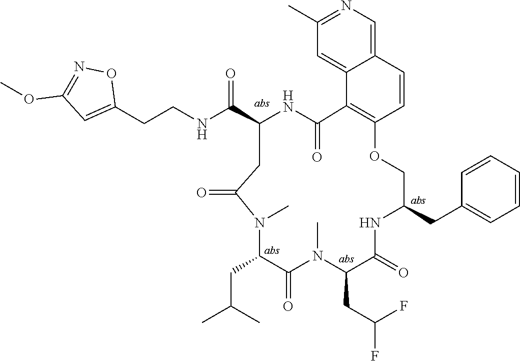 [SMILES: COc1cc(CCNC(=O)[C@@H]2CC(=O)N(C)[C@@H](CC(C)C)C(=O)N(C)[C@H](CC(F)F)C(=O)N[C@H](Cc3ccccc3)COc3ccc4cnc(C)cc4c3C(=O)N2)on1]

26

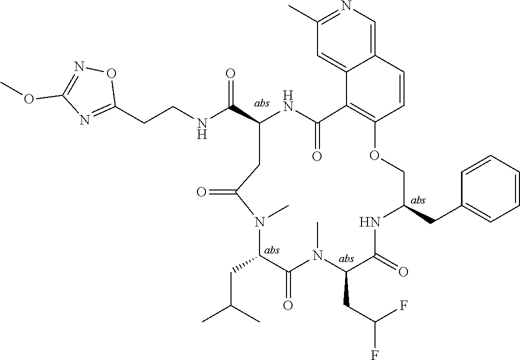 [SMILES: COc1noc(CCNC(=O)[C@@H]2CC(=O)N(C)[C@@H](CC(C)C)C(=O)N(C)[C@H](CC(F)F)C(=O)N[C@H](Cc3ccccc3)COc3ccc4cnc(C)cc4c3C(=O)N2)n1]

27

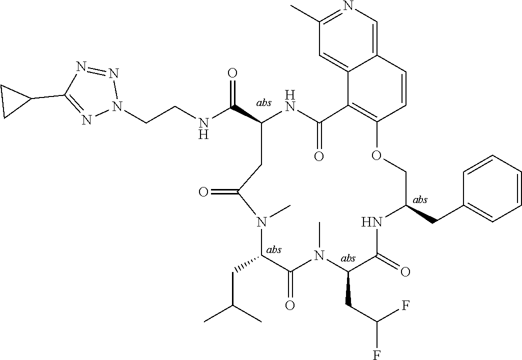 [SMILES: Cc1cc2c3c(ccc2cn1)OC[C@@H](Cc1ccccc1)NC(=O)[C@@H](CC(F)F)N(C)C(=O)[C@H](CC(C)C)N(C)C(=O)C[C@@H](C(=O)NCCn1nnc(C2CC2)n1)NC3=O]

28

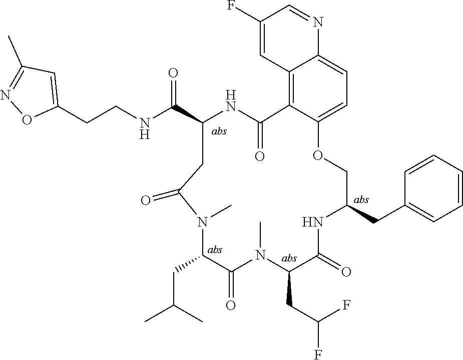 [SMILES: Cc1cc(CCNC(=O)[C@@H]2CC(=O)N(C)[C@@H](CC(C)C)C(=O)N(C)[C@H](CC(F)F)C(=O)N[C@H](Cc3ccccc3)COc3ccc4ncc(F)cc4c3C(=O)N2)on1]

29

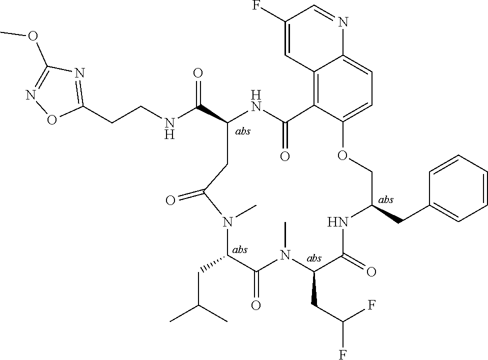 [SMILES: COc1noc(CCNC(=O)[C@@H]2CC(=O)N(C)[C@@H](CC(C)C)C(=O)N(C)[C@H](CC(F)F)C(=O)N[C@H](Cc3ccccc3)COc3ccc4ncc(F)cc4c3C(=O)N2)n1]

30

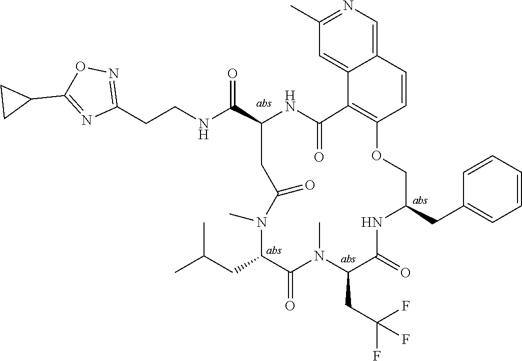 [SMILES: Cc1cc2c3c(ccc2cn1)OC[C@@H](Cc1ccccc1)NC(=O)[C@@H](CC(F)(F)F)N(C)C(=O)[C@H](CC(C)C)N(C)C(=O)C[C@@H](C(=O)NCCc1noc(C2CC2)n1)NC3=O]

31

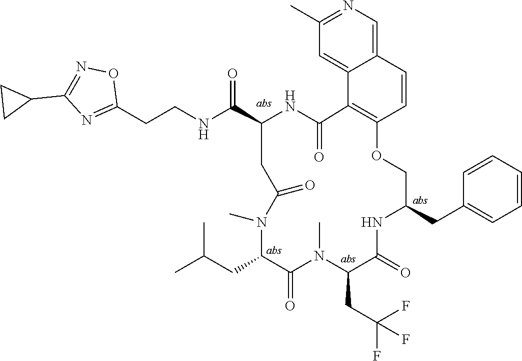 [SMILES: Cc1cc2c3c(ccc2cn1)OC[C@@H](Cc1ccccc1)NC(=O)[C@@H](CC(F)(F)F)N(C)C(=O)[C@H](CC(C)C)N(C)C(=O)C[C@@H](C(=O)NCCc1nc(C2CC2)no1)NC3=O]

32

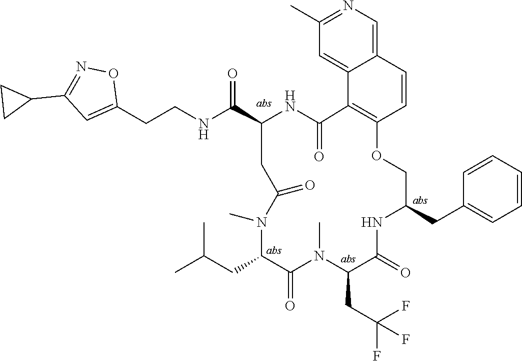 [SMILES: Cc1cc2c3c(ccc2cn1)OC[C@@H](Cc1ccccc1)NC(=O)[C@@H](CC(F)(F)F)N(C)C(=O)[C@H](CC(C)C)N(C)C(=O)C[C@@H](C(=O)NCCc1cc(C2CC2)no1)NC3=O]

33

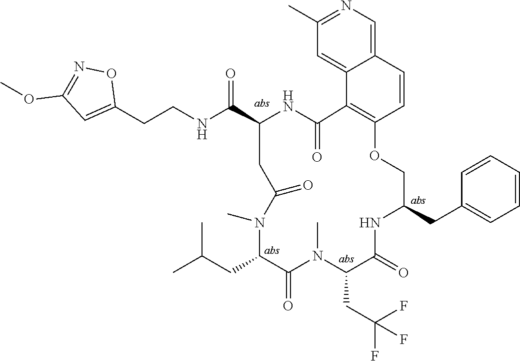 [SMILES: COc1cc(CCNC(=O)[C@@H]2CC(=O)N(C)[C@@H](CC(C)C)C(=O)N(C)[C@@H](CC(F)(F)F)C(=O)N[C@H](Cc3ccccc3)COc3ccc4cnc(C)cc4c3C(=O)N2)on1]

34

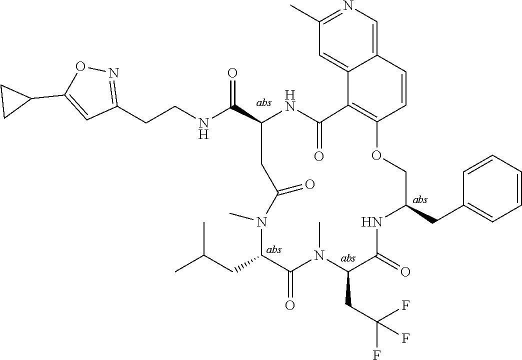 [SMILES: Cc1cc2c3c(ccc2cn1)OC[C@@H](Cc1ccccc1)NC(=O)[C@@H](CC(F)(F)F)N(C)C(=O)[C@H](CC(C)C)N(C)C(=O)C[C@@H](C(=O)NCCc1cc(C2CC2)on1)NC3=O]

35

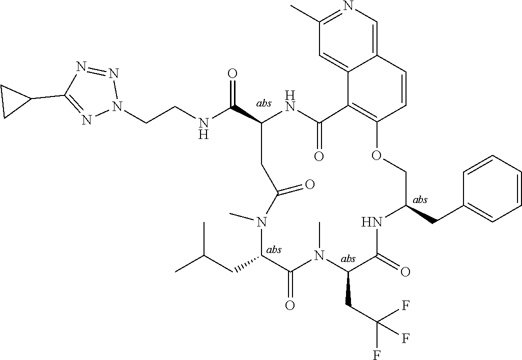 [SMILES: Cc1cc2c3c(ccc2cn1)OC[C@@H](Cc1ccccc1)NC(=O)[C@@H](CC(F)(F)F)N(C)C(=O)[C@H](CC(C)C)N(C)C(=O)C[C@@H](C(=O)NCCn1nnc(C2CC2)n1)NC3=O]

36

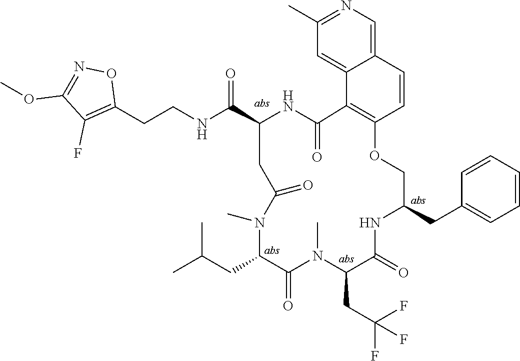 [SMILES: COc1noc(CCNC(=O)[C@@H]2CC(=O)N(C)[C@@H](CC(C)C)C(=O)N(C)[C@H](CC(F)(F)F)C(=O)N[C@H](Cc3ccccc3)COc3ccc4cnc(C)cc4c3C(=O)N2)c1F]

37

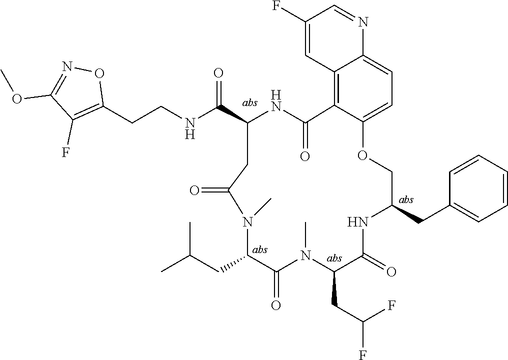 [SMILES: COc1noc(CCNC(=O)[C@@H]2CC(=O)N(C)[C@@H](CC(C)C)C(=O)N(C)[C@H](CC(F)F)C(=O)N[C@H](Cc3ccccc3)COc3ccc4ncc(F)cc4c3C(=O)N2)c1F]

38

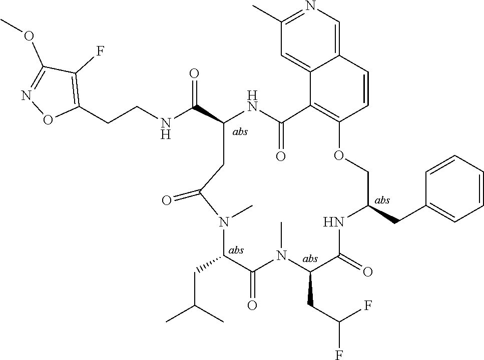 [SMILES: COc1noc(CCNC(=O)[C@@H]2CC(=O)N(C)[C@@H](CC(C)C)C(=O)N(C)[C@H](CC(F)F)C(=O)N[C@H](Cc3ccccc3)COc3ccc4cnc(C)cc4c3C(=O)N2)c1F]

39

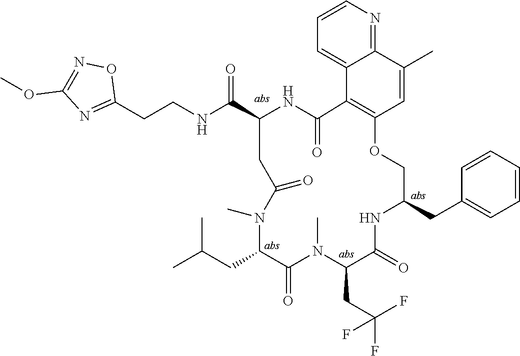 [SMILES: COc1noc(CCNC(=O)[C@@H]2CC(=O)N(C)[C@@H](CC(C)C)C(=O)N(C)[C@H](CC(F)(F)F)C(=O)N[C@H](Cc3ccccc3)COc3cc(C)c4ncccc4c3C(=O)N2)n1]

40

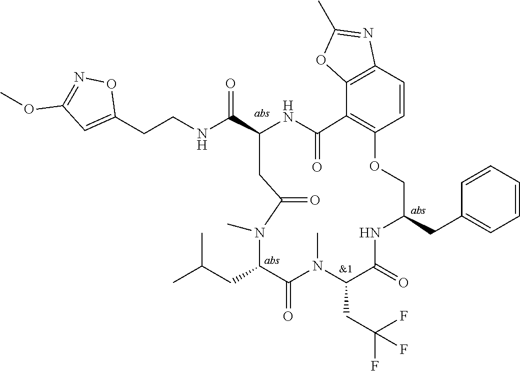 [SMILES: COc1cc(CCNC(=O)[C@@H]2CC(=O)N(C)[C@@H](CC(C)C)C(=O)N(C)[C@@H](CC(F)(F)F)C(=O)N[C@H](Cc3ccccc3)COc3ccc4nc(C)oc4c3C(=O)N2)on1]

41

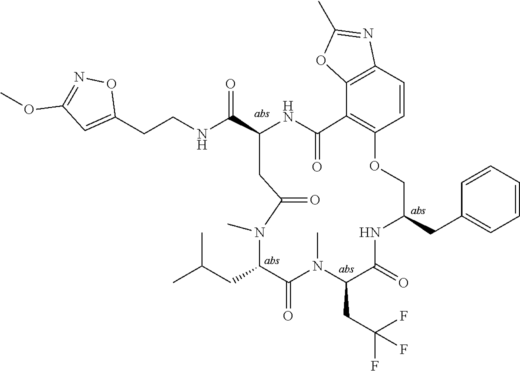 [SMILES: COc1cc(CCNC(=O)[C@@H]2CC(=O)N(C)[C@@H](CC(C)C)C(=O)N(C)[C@H](CC(F)(F)F)C(=O)N[C@H](Cc3ccccc3)COc3ccc4nc(C)oc4c3C(=O)N2)on1]

42

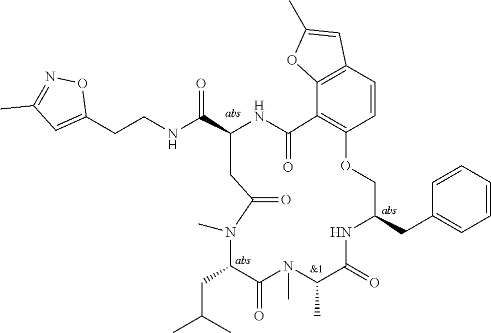 [SMILES: Cc1cc(CCNC(=O)[C@@H]2CC(=O)N(C)[C@@H](CC(C)C)C(=O)N(C)[C@@H](C)C(=O)N[C@H](Cc3ccccc3)COc3ccc4cc(C)oc4c3C(=O)N2)on1]

43

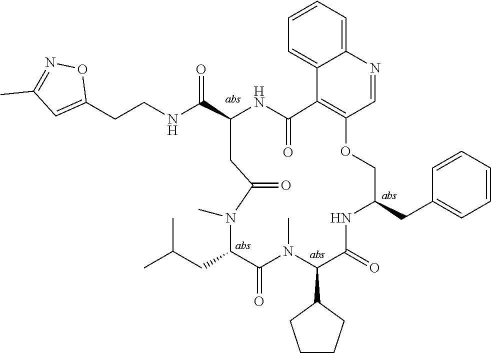 [SMILES: Cc1cc(CCNC(=O)[C@@H]2CC(=O)N(C)[C@@H](CC(C)C)C(=O)N(C)[C@H](C3CCCC3)C(=O)N[C@H](Cc3ccccc3)COc3cnc4ccccc4c3C(=O)N2)on1]

44

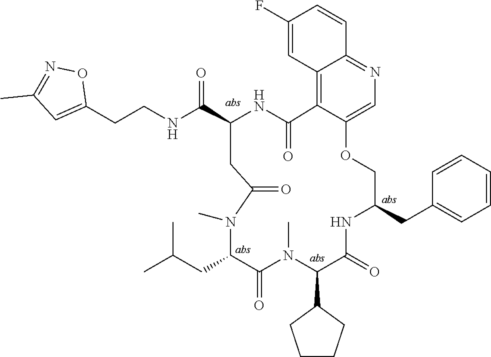 [SMILES: Cc1cc(CCNC(=O)[C@@H]2CC(=O)N(C)[C@@H](CC(C)C)C(=O)N(C)[C@H](C3CCCC3)C(=O)N[C@H](Cc3ccccc3)COc3cnc4ccc(F)cc4c3C(=O)N2)on1]

45

In Table S above, a stereogenic or asymmetric center indicated in the structures as “abs” represents said stereogenic or asymmetric center in the respective enantiomerically enriched absolute (R)- or (S)-configuration as depicted. A stereogenic or asymmetric center indicated in the structures as “&1” represents said stereogenic or asymmetric center in the respective (RS)-configuration, i.e. comprising the respective enantiomerically enriched (R)-configuration, or enantiomerically enriched(S)-configuration, or any mixture of epimers at such center.

### II. Biological Assays

Compounds of the present invention may be further characterized with regard to their general pharmacokinetic and pharmacological properties using conventional assays well known in the art for example relating to their bioavailablility in different species (such as rat or dog); or for their properties with regard to drug safety and/or toxicological properties using conventional assays well known in the art, for example relating to cytochrome P450 enzyme inhibition and time dependent inhibition, pregnane X receptor (PXR) activation, glutathione binding, or phototoxic behavior.

### Biological In Vitro Assays

Evaluation of Compound EC₅₀ and E_{Max} Values

The corrector activities of the compounds of formula (I) on CFTR are determined in accordance with the following experimental method. The method measures the effect of over-night compound incubation on F508del-CFTR cell surface expression in a recombinant U2OS cell line (DiscoveRx, #93-0987C3). This cell line is engineered to co-express (i) human F508del-CFTR tagged with a Prolink (PK=short β-galactosidase fragment) and (ii) the remainder of the β-galactosidase enzyme (Enzyme Acceptor; EA) localized to the plasma membrane. Incubation with compounds that increase PK-tagged F508del-CFTR at the plasma membrane will lead to complementation of the EA fragment to form a functional β-galactosidase enzyme which is quantified by a chemiluminescence reaction.

Briefly, the cells are seeded at 3500 cells/well into 384-well low volume plates (Corning, #3826) in 20 μl of full medium (Mc Coy's 5a (#36600-021, Gibco)+10% FBS Gibco+penicillin/streptomycin). The cells are incubated for 5 h in the incubator before the addition of 5 μl/well of compound dilution series (5× working stocks in full medium). Final DMSO concentration in the assay is 0.25%. The cells are co-incubated with the compounds for 16 h in the incubator at 37° C., 5% CO₂. The next day, the cell plates are incubated for 2 h at RT in the dark. Then, 10 μl/well of Flash detection reagent (DiscoverX, #93-0247) is added, the plate is incubated for another 30 min at RT in the dark and chemiluminescence is measured. Concentration-response curves are generated using compound-intrinsic maximal efficacy as upper plateau, and from these CRCs compound-intrinsic EC₅₀ values are determined. Compound-specific Eₘₐₓ values are calculated in relation to the Eₘₐₓ of the corrector lumacaftor (Eₘₐₓ lumacaftor=100%).

The calculated EC₅₀ values may fluctuate depending on the daily assay performance. Fluctuations of this kind are known to those skilled in the art. EC₅₀ values from several measurements are given as geomean values. The calculated Eₘₐₓ values may fluctuate depending on the daily assay performance. Fluctuations of this kind are known to those skilled in the art. Eₘₐₓ values from several measurements are given as arithmetic mean values.

Table of Biological Data:

| | EC₅₀ | Emax |
|---|---|---|
| Ex. | [nmol/l] | [%] |
| 1 | 263 | 490 |
| 2 | 737 | 288 |
| 3 | 146 | 460 |
| 4 | 673 | 298 |
| 5 | 281 | 330 |
| 6 | 106 | 326 |
| 7 | 423 | 306 |
| 8 | 308 | 319 |
| 9 | 299 | 338 |
| 10 | 156 | 337 |
| 11 | 190 | 323 |
| 12 | 675 | 358 |
| 13 | 650 | 350 |
| 14 | 399 | 308 |
| 15 | 197 | 324 |
| 16 | 258 | 335 |
| 17 | 266 | 352 |
| 18 | 149 | 330 |
| 19 | 723 | 200 |
| 20 | 248 | 355 |
| 21 | 276 | 431 |
| 22 | 674 | 354 |
| 23 | 353 | 388 |
| 24 | 283 | 340 |
| 25 | 216 | 394 |
| 26 | 275 | 459 |
| 27 | 373 | 491 |
| 28 | 399 | 485 |
| 29 | 447 | 404 |
| 30 | 273 | 452 |
| 31 | 201 | 464 |
| 32 | 165 | 405 |
| 33 | 508 | 412 |
| 34 | 185 | 465 |
| 35 | 338 | 439 |
| 36 | 222 | 413 |
| 37 | 85 | 507 |
| 38 | 67 | 431 |
| 39 | 109 | 463 |
| 40 | 119 | 497 |
| 41 | 703 | 440 |
| 42 | 601 | 482 |
| 43 | 523 | 268 |
| 44 | 149 | 280 |
| 45 | 142 | 328 |

Example of Restoration of Cell Surface F508Del-CFTR Expression by Compounds of Formula (I) in Combination with CFTR Correctors Having Different Mechanisms

The restoration of F508del-CFTR cell surface expression by Example COMPOUND 3 in combination with CFTR correctors having different mechanisms can be determined in vitro using the DiscoveRx enzyme fragment complementation assay.

### Combination Efficacy Experiment: Restauration of F508Del-CFTR Cell Surface Expression in a Recombinant U20S Cell Line

This assay uses above-mentioned U2OS cells which express the human F508del-CFTR chloride channel (DiscoveRx, #93-0987C3). Cells are seeded at 3500 cells/well into 384-well low volume plates (Corning, #3826) in 20 μl of full medium (Mc Coy's 5a (#36600-021, Gibco)+10% FBS Gibco+penicillin/streptomycin). The cells are incubated for 5 h in the incubator before the addition of 5 μl/well of compound dilutions (5× working stocks in full medium) to reach the indicated concentrations. Final DMSO concentration in the assay is 0.34%. The cells are co-incubated with the compounds for 16 h at 37° C., 5% CO₂. The next day, the cell plates are incubated for 2 h at RT in the dark. Then, 10 μl/well of Flash detection reagent (DiscoverX, #93-0247) are added, the plate is incubated for another 30 min at RT in the dark and chemiluminescence is measured. Measured values are expressed as fold-change versus the baseline value (absence of CFTR correctors=vehicle treatment). The results from the combination efficacy experiments are shown in FIG. 1.

FIG. 1 shows the analysis of F508del-CFTR cell surface expression as a consequence of treatment with 200 nM of Example COMPOUND 3 when added to a basal treatment of CFTR correctors of different mechanisms, such as 2 uM tezacaftor or 0.2 uM galicaftor (both type-I correctors), or 5 uM Corrector 4a (type-II corrector) or 2 uM elexacaftor (type-III corrector) or the combination of [2 uM tezacaftor+2 uM elexacaftor] (type-I corrector+type-III corrector). Example COMPOUND 3 enhances F508del-CFTR surface expression on top of all basal treatments. Error bars represent the standard error of the mean.

FIG. 6 shows the analysis of F508del-CFTR cell surface expression as a consequence of treatment with 200 nM of Example COMPOUND 3 when added to a basal treatment of CFTR correctors of type-III corrector mechanism, such as 2 uM elexacaftor or 5 uM of the compound of example 2 of WO 2019/071078 (PTI-801). Example COMPOUND 3 enhances F508del-CFTR surface expression on top of these basal treatments. Adding one type-III corrector (elexacaftor; 2 uM) to another type-III corrector (PTI-801; 5 uM) does not lead to additive effects. Error bars represent the standard error of the mean.

Example of Restoration of F508Del-CFTR Function by Compounds of Formula (I) in Combination with CFTR Correctors Having Different Mechanisms and CFTR Potentiators

The restoration of F508del-CFTR function by Example COMPOUND 3 in combination with CFTR correctors having different mechanisms and CFTR potentiators can be determined in vitro using the YFP quenching assay.

### Combination Efficacy Experiment: Restauration of F508Del-CFTR Function in a Recombinant U2OS Cell Line

This assay uses U2OS cells which express the human F508del-CFTR chloride channel (DiscoveRx, #93-0987C3) and in addition a halide-sensitive mutant form of yellow fluorescent protein (YFP Topaz F46L/H₁₄₈Q/1152L; US 2006/0257934 A1). This mutant YFP reports the entry of exogenously added iodide (surrogate ion) through functional surface CFTR based on the YFP's property to bind iodide which leads to quenching of its fluorescence.

Cells are seeded at 20000 cells/well into 384-well in 384w black clear bottom plate in 40 μL/well growth medium (Mc Coy's 5a (#36600-021, Gibco)+10% FBS Gibco+penicillin/streptomycin), containing the various CFTR correctors at the indicated concentrations. Final DMSO concentration in the assay is 0.1%. The cells are co-incubated with the compounds for 24 h at 37° C., 5% CO₂. The next day, plates are washed twice with 55 uL/well of PBS+ (PBS containing 0.9 mM Ca²⁺, and 0.5 mM Mg²⁺). PBS+ is fully removed and cells are supplemented with 15 uL PBS+. Then, 5 μL of 4× concentrated stocks of potentiators or vehicle in dilution buffer (PBS+, 0.4 uM forskolin, 0.2% bovine serum albumin (fatty-acid free), pH 7.4) are added and incubated for 30 min in the dark. Final DMSO concentration in the assay is 0.5%. Then, plates are transferred to the FLIPR Tetra (fluorescence imaging plate reader, Molecular Devices: excitation 470-495 nm; emission: 526-585 nm), baseline fluorescence reading of the YFP signal is performed for 6 sec (10×0.6 second intervals) after which 25 uL of iodide buffer (137 mM NaI; 2.7 mM KCl; 1.5 mM KH₂PO₄; 8.1 mM Na₂HPO₄, 1 mM CaCl₂); 0.5 mM MgCl₂. pH 7.4) are added and fluorescence reading is continued for 70 seconds (50×0.6 second intervals; 20×2 second intervals) to assess YFP quenching through CFTR-mediated iodide influx. For analysis, fluorescence traces are aligned and normalized at the last time point before iodide addition (normalized fluorescence=1). Normalized fluorescence values obtained 14 seconds after the iodide addition are used to assess the degree of YFP quenching, i.e. CFTR function.

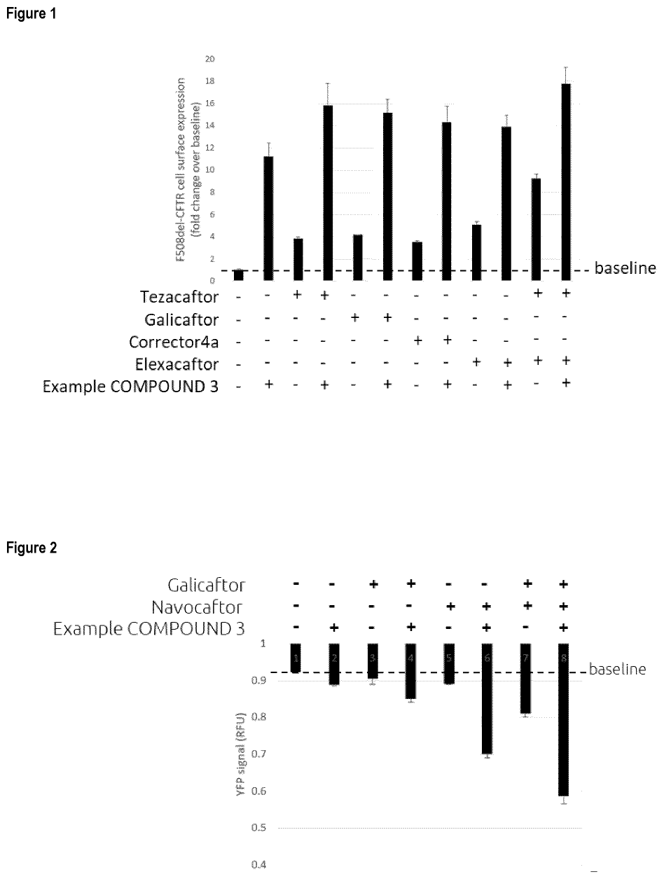

The results from the combination efficacy experiments are shown in FIGS. 2-5.

FIG. 2 shows the analysis of F508del-CFTR function (YFP quenching assay) as a consequence of treatment with 400 nM of Example COMPOUND 3 when added to a basal treatment of type-I CFTR corrector galicaftor (1 uM) and/or CFTR potentiator navocaftor (50 nM). Example COMPOUND 3 enhances F508del-CFTR function on top of all basal treatments. Error bars represent the standard error of the mean.

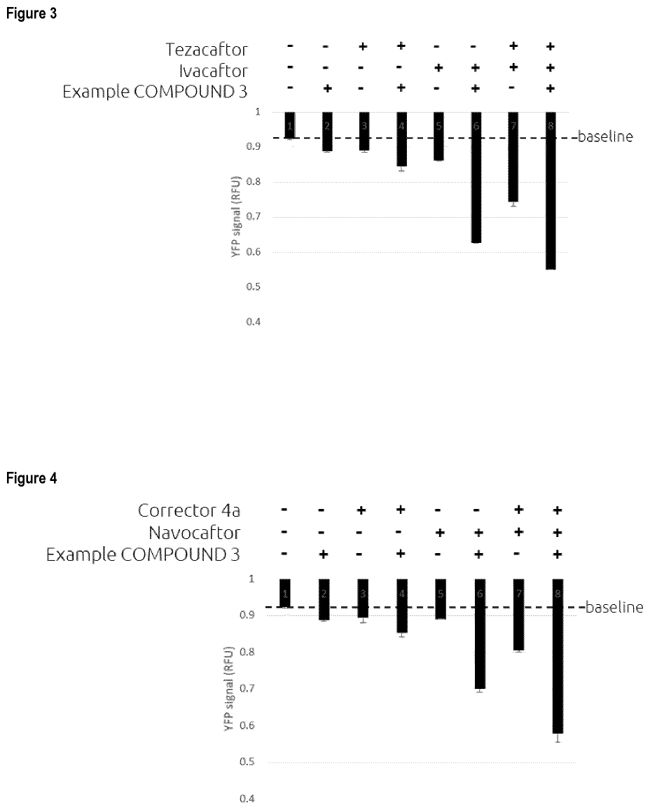

FIG. 3 shows the analysis of F508del-CFTR function (YFP quenching assay) as a consequence of treatment with 400 nM of Example COMPOUND 3 when added to a basal treatment of type-I CFTR corrector tezacaftor (2 uM) and/or CFTR potentiator ivacaftor (2 nM). Example COMPOUND 3 enhances F508del-CFTR function on top of all basal treatments. Error bars represent the standard error of the mean.

FIG. 4 shows the analysis of F508del-CFTR function (YFP quenching assay) as a consequence of treatment with 400 nM of Example COMPOUND 3 when added to a basal treatment of type-II CFTR corrector Corrector 4a (5 uM) and/or CFTR potentiator navocaftor (50 nM). Example COMPOUND 3 enhances F508del-CFTR function on top of all basal treatments. Error bars represent the standard error of the mean.

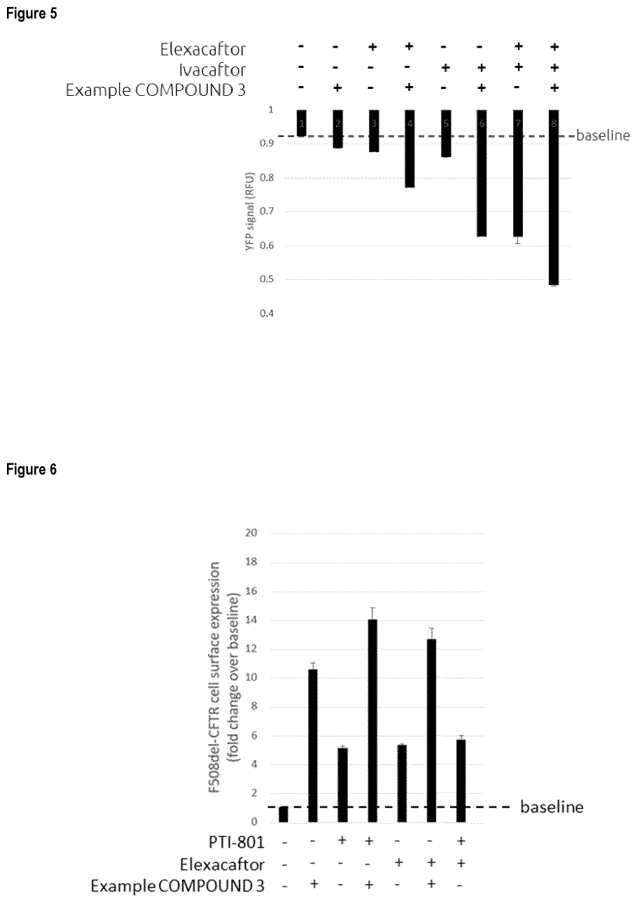

FIG. 5 shows the analysis of F508del-CFTR function (YFP quenching assay) as a consequence of treatment with 400 nM of Example COMPOUND 3 when added to a basal treatment of type-III CFTR corrector elexacaftor (2 uM) and/or CFTR potentiator ivacaftor (2 nM). Example COMPOUND 3 enhances F508del-CFTR function on top of all basal treatments. Error bars represent the standard error of the mean.

### REFERENCES

Boyle M P et al (2014). Lancet Respir Med 2(7):527-538.
Dalemans W et al (1991). Nature 354:526-528.
Davies J C et al (2018). N Engl J Med 379(17):1599-1611
de Boeck K (2020). Acta Paediatr 109(5):893-895
Elborn J S (2016). Lancet 388:2519-2531.
Fiedorczuk K et al (2022). Cell 185(1):158-168.
Flores A M et al (2016). FASEB J 30(5):1789-1797.
Galietta L V et al (2001). Am J Physiol Cell Physiol 281(5):C1734-1742.
Hutt D M et al (2011). ACS Med Chem Lett 2(9):703-707.
Kazani S et al (2021). J Cyst Fibros 20(2):250-256.
Keating D et al (2018). N Engl J Med 379(17):1612-1620.
Le Grand D et al (2021). J Med Chem 64(11):7241-7260.
Marchesin V et al (2023). //doi.org/10.1101/2023.08.15.552928
Okiyoneda T et al (2013). Nat Chem Biol 9(7):444-454.
Patel S D et al (2020). Eur Respir Rev 29(156):190068.
Rowe S M et al (2020). Int J Chron Obstruct Pulmon Dis 15:2399-2409.
Stevers L M et al (2022). Nat Commun 13:3586.
Van Goor F et al (2011). Proc Natl Acad Sci USA 108(46):18843-18848.
Veit G et al (2018). Nat Med 24(11):1732-1742.
Veit G et al (2020). JCI Insight 5(18):e139983.
Wang X et al (2018). J Med Chem 61(4):1436-1449.

1. A pharmaceutical composition comprising, as active principles, a compound of Formula (I): 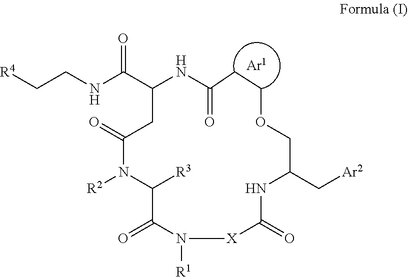
wherein
X represents —CR^{X1}R^{X2}, wherein R^{X1} represents hydrogen, and R^{X2} represents
C₁₋₆-alkyl;
C₁₋₄-fluoroalkyl; or
C₃₋₆-cycloalkyl;
R¹ represents C₁₋₄-alkyl;
R² represents C₁₋₄-alkyl;
R³ represents C₁₋₆-alkyl;
R⁴ represents 5-membered heteroaryl, wherein said 5-membered heteroaryl independently is unsubstituted, mono-, or di-substituted, wherein the substitutents are independently selected from C₁₋₄-alkyl, C₁₋₄-alkoxy, C₁₋₃-fluoroalkyl, C₁₋₃-fluoroalkoxy, C₃₋₆-cycloalkyl, or halogen;
Ar¹ represents 8- to 10-membered bicyclic heteroarylene, wherein said bicyclic heteroarylene independently is unsubstituted or mono-substituted with C₁₋₄-alkyl, or halogen;
Ar² represents phenyl, wherein said phenyl is unsubstituted, mono- or di-substituted wherein the substituents are independently selected from C₁₋₄-alkyl, C₁₋₃-fluoroalkyl, halogen, C₁₋₆-alkoxy, and C₁₋₃-fluoroalkoxy;
or a pharmaceutically acceptable salt thereof;
in combination with one or more therapeutically active ingredients acting as CFTR modulator(s); wherein said CFTR modulator(s) is/are one or more CFTR corrector(s), and/or a CFTR potentiator; or a pharmaceutically acceptable salt thereof; as well as at least one pharmaceutically acceptable excipient.

2. A pharmaceutical composition according to claim 1, wherein the compound of Formula (I) is a compound of Formula (I_{E}): 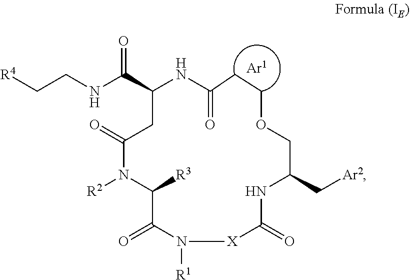
or a pharmaceutically acceptable salt thereof.

3. A pharmaceutical composition according to claim 1, wherein X represents —CR^{X1}R^{X2}, wherein R^{X1} is hydrogen, and R^{X2} is 2,2,2-trifluoroethyl.

4. A pharmaceutical composition according to claim 1, wherein R⁴ represents oxadiazolyl, wherein said 5-membered heteroaryl independently is mono-substituted with C₁₋₄-alkoxy.

5. A pharmaceutical composition according to claim 1, wherein Ar¹ represents 10-membered bicyclic heteroarylene, wherein said bicyclic heteroarylene is mono-substituted with halogen.

6. A pharmaceutical composition according to claim 1, wherein Ar² represents unsubstituted phenyl.

7. A pharmaceutical composition according to claim 1, wherein the compound of Formula (I) is:
(3S,7S,10R,13R)-13-benzyl-7-isobutyl-N-(2-(3-methoxy-1,2,4-oxadiazol-5-yl)ethyl)-6,9,20-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10S,13R)-13-benzyl-7-isobutyl-N-(2-(3-methoxy-1,2,4-oxadiazol-5-yl)ethyl)-6,9,20-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10S,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-7-isobutyl-10-isopropyl-6,9-dimethyl-N-(2-(3-methylisoxazol-5-yl)ethyl)-1,5,8,11-tetraoxo-1,2,3,4,5,6,7,8,9,10,11,12,13,14-tetradecahydro-[1]oxa[4,7,10,14]tetraazacycloheptadecino[17,16-c]quinoline-3-carboxamide;
(3S,7S,10R,13R)-13-benzyl-7,10-diisobutyl-6,9-dimethyl-N-(2-(3-methylisoxazol-5-yl)ethyl)-1,5,8,11-tetraoxo-1,2,3,4,5,6,7,8,9,10,11,12,13,14-tetradecahydro-[1]oxa[4,7,10,14]tetraazacycloheptadecino[17,16-c]quinoline-3-carboxamide;
(3S,7S,10R,13R)-13-benzyl-7-isobutyl-6,9,10-trimethyl-N-(2-(3-methylisoxazol-5-yl)ethyl)-1,5,8,11-tetraoxo-1,2,3,4,5,6,7,8,9,10,11,12,13,14-tetradecahydro-[1]oxa[4,7,10,14]tetraazacycloheptadecino[17,16-c]quinoline-3-carboxamide;
(3S,7S,10R,13R)-13-benzyl-N-(2-(3-cyclopropylisoxazol-5-yl)ethyl)-7-isobutyl-6,9,10-trimethyl-1,5,8,11-tetraoxo-1,2,3,4,5,6,7,8,9,10,11,12,13,14-tetradecahydro-[1]oxa[4,7,10,14]tetraazacycloheptadecino[17,16-c]quinoline-3-carboxamide;
(3S,7S,10R,13R)-13-benzyl-10-(2,2-difluoroethyl)-7-isobutyl-6,9-dimethyl-N-(2-(3-methylisoxazol-5-yl)ethyl)-1,5,8,11-tetraoxo-1,2,3,4,5,6,7,8,9,10,11,12,13,14-tetradecahydro-[1]oxa[4,7,10,14]tetraazacycloheptadecino[17,16-c]quinoline-3-carboxamide;
(3S,7S,10R,13R)-13-benzyl-20-fluoro-7-isobutyl-N-(2-(3-methoxyisoxazol-5-yl)ethyl)-6,9,10-trimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-N-(2-(4-cyclopropyl-2H-1,2,3-triazol-2-yl)ethyl)-20-fluoro-7-isobutyl-6,9,10-trimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-7-isobutyl-N-(2-(3-methoxyisoxazol-5-yl)ethyl)-6,9,10-trimethyl-1,5,8,11-tetraoxo-1,2,3,4,5,6,7,8,9,10,11,12,13,14-tetradecahydroimidazo[1′,2′:1,6]pyrido[2,3-p][1]oxa[4,7,10,14]tetraazacycloheptadecine-3-carboxamide;
(3S,7S,10R,13R)-13-benzyl-N-(2-(3-cyclopropylisoxazol-5-yl)ethyl)-7-isobutyl-6,9,10-trimethyl-1,5,8,11-tetraoxo-1,2,3,4,5,6,7,8,9,10,11,12,13,14-tetradecahydroimidazo[1′,2′:1,6]pyrido[2,3-p][1]oxa[4,7,10,14]tetraazacycloheptadecine-3-carboxamide;
(3S,7S,10R,13R)-13-benzyl-N-(2-(3-cyclopropylisoxazol-5-yl)ethyl)-20-fluoro-7-isobutyl-6,9,10-trimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-N-(2-(5-cyclopropylisoxazol-3-yl)ethyl)-20-fluoro-7-isobutyl-6,9,10-trimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-N-(2-(5-cyclopropyl-1,2,4-oxadiazol-3-yl)ethyl)-20-fluoro-7-isobutyl-6,9,10-trimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-N-(2-(3-cyclopropyl-1,2,4-oxadiazol-5-yl)ethyl)-20-fluoro-7-isobutyl-6,9,10-trimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-N-(2-(5-cyclopropyl-1,2,4-oxadiazol-3-yl)ethyl)-10-(2,2-difluoroethyl)-20-fluoro-7-isobutyl-6,9-dimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10S,13R)-13-benzyl-N-(2-(5-cyclopropyl-1,2,4-oxadiazol-3-yl)ethyl)-10-(2,2-difluoroethyl)-20-fluoro-7-isobutyl-6,9-dimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-N-(2-(5-cyclopropyl-1,2,4-oxadiazol-3-yl)ethyl)-10-ethyl-20-fluoro-7-isobutyl-6,9-dimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-10-(2,2-difluoroethyl)-20-fluoro-7-isobutyl-N-(2-(3-methoxyisoxazol-5-yl)ethyl)-6,9-dimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-7-isobutyl-6,9,10,20-tetramethyl-N-(2-(3-methylisoxazol-5-yl)ethyl)-1,5,8,11-tetraoxo-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-7-isobutyl-N-(2-(3-methoxyisoxazol-5-yl)ethyl)-6,9,10,20-tetramethyl-1,5,8,11-tetraoxo-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-N-(2-(3-cyclopropyl-1,2,4-oxadiazol-5-yl)ethyl)-7-isobutyl-6,9,10,20-tetramethyl-1,5,8,11-tetraoxo-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-N-(2-(5-cyclopropyl-2H-tetrazol-2-yl)ethyl)-10-(2,2-difluoroethyl)-20-fluoro-7-isobutyl-6,9-dimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-10-(2,2-difluoroethyl)-7-isobutyl-N-(2-(3-methoxyisoxazol-5-yl)ethyl)-6,9,20-trimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-10-(2,2-difluoroethyl)-7-isobutyl-N-(2-(3-methoxy-1,2,4-oxadiazol-5-yl)ethyl)-6,9,20-trimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-N-(2-(5-cyclopropyl-2H-tetrazol-2-yl)ethyl)-10-(2,2-difluoroethyl)-7-isobutyl-6,9,20-trimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-10-(2,2-difluoroethyl)-20-fluoro-7-isobutyl-6,9-dimethyl-N-(2-(3-methylisoxazol-5-yl)ethyl)-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-10-(2,2-difluoroethyl)-20-fluoro-7-isobutyl-N-(2-(3-methoxy-1,2,4-oxadiazol-5-yl)ethyl)-6,9-dimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-N-(2-(5-cyclopropyl-1,2,4-oxadiazol-3-yl)ethyl)-7-isobutyl-6,9,20-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-N-(2-(3-cyclopropyl-1,2,4-oxadiazol-5-yl)ethyl)-7-isobutyl-6,9,20-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-N-(2-(3-cyclopropylisoxazol-5-yl)ethyl)-7-isobutyl-6,9,20-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-7-isobutyl-N-(2-(3-methoxyisoxazol-5-yl)ethyl)-6,9,20-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-N-(2-(5-cyclopropylisoxazol-3-yl)ethyl)-7-isobutyl-6,9,20-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-N-(2-(5-cyclopropyl-2H-tetrazol-2-yl)ethyl)-7-isobutyl-6,9,20-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-N-(2-(4-fluoro-3-methoxyisoxazol-5-yl)ethyl)-7-isobutyl-6,9,20-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-10-(2,2-difluoroethyl)-20-fluoro-N-(2-(4-fluoro-3-methoxyisoxazol-5-yl)ethyl)-7-isobutyl-6,9-dimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]quinoline-3-carboxamide;
(3S,7S,10R,13R)-13-benzyl-10-(2,2-difluoroethyl)-N-(2-(4-fluoro-3-methoxyisoxazol-5-yl)ethyl)-7-isobutyl-6,9,20-trimethyl-1,5,8,11-tetraoxo-1,2,3,4,5,6,7,8,9,10,11,12,13,14-tetradecahydro-[1]oxa[4,7,10,14]tetraazacycloheptadecino[16,17-f]isoquinoline-3-carboxamide;
(3S,7S,10R,13R)-13-benzyl-7-isobutyl-N-(2-(3-methoxy-1,2,4-oxadiazol-5-yl)ethyl)-6,9,17-trimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide;
(8R,11RS,14S,18S)-8-benzyl-14-isobutyl-N-(2-(3-methoxyisoxazol-5-yl)ethyl)-2,12,15-trimethyl-10,13,16,20-tetraoxo-11-(2,2,2-trifluoroethyl)-7,8,9,10,11,12,13,14,15,16,17,18,19,20-tetradecahydrooxazolo[4′,5′:5,6]benzo[1,2-p][1]oxa[4,7,10,14]tetraazacycloheptadecine-18-carboxamide;
(8R,11R,14S,18S)-8-benzyl-14-isobutyl-N-(2-(3-methoxyisoxazol-5-yl)ethyl)-2,12,15-trimethyl-10,13,16,20-tetraoxo-11-(2,2,2-trifluoroethyl)-7,8,9,10,11,12,13,14,15,16,17,18,19,20-tetradecahydrooxazolo[4′,5′:5,6]benzo[1,2-p][1]oxa[4,7,10,14]tetraazacycloheptadecine-18-carboxamide;
(8R,11RS,14S,18S)-8-benzyl-14-isobutyl-2,11,12,15-tetramethyl-N-(2-(3-methylisoxazol-5-yl)ethyl)-10,13,16,20-tetraoxo-7,8,9,10,11,12,13,14,15,16,17,18,19,20-tetradecahydrobenzofuro[7,6-p][1]oxa[4,7,10,14]tetraazacycloheptadecine-18-carboxamide;
(3S,7S,10R,13R)-13-benzyl-10-cyclopentyl-7-isobutyl-6,9-dimethyl-N-(2-(3-methylisoxazol-5-yl)ethyl)-1,5,8,11-tetraoxo-1,2,3,4,5,6,7,8,9,10,11,12,13,14-tetradecahydro-[1]oxa[4,7,10,14]tetraazacycloheptadecino[17,16-c]quinoline-3-carboxamide; or
(3S,7S,10R,13R)-13-benzyl-10-cyclopentyl-20-fluoro-7-isobutyl-6,9-dimethyl-N-(2-(3-methylisoxazol-5-yl)ethyl)-1,5,8,11-tetraoxo-1,2,3,4,5,6,7,8,9,10,11,12,13,14-tetradecahydro-[1]oxa[4,7,10,14]tetraazacycloheptadecino[17,16-c]quinoline-3-carboxamide;
or a pharmaceutically acceptable salt thereof.

8. A pharmaceutical composition comprising, as active principles, (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide;
or a pharmaceutically acceptable salt thereof;
in combination with one or more therapeutically active ingredients acting as CFTR modulator(s); wherein said CFTR modulator(s) is/are one or more CFTR corrector(s), and/or a CFTR potentiator; or a pharmaceutically acceptable salt thereof; as well as at least one pharmaceutically acceptable excipient.

9. A pharmaceutical composition according to claim 1, wherein the one or more therapeutically active ingredients acting as CFTR modulator(s) is/are a type-I corrector selected from lumacaftor, tezacaftor, and galicaftor; and/or a type-II corrector which is Corrector4a; and/or a type-III corrector selected from elexacaftor and vanzacaftor; and/or a CFTR potentiator selected from ivacaftor, navocaftor, icenticaftor, and deutivacaftor; or a pharmaceutically acceptable salt thereof.

10. A pharmaceutical composition according to claim 1, wherein said composition comprises the compound of formula (I), or a pharmaceutically acceptable salt thereof, and one therapeutically active ingredient acting as CFTR modulator, wherein said CFTR modulator is a CFTR potentiator; or a pharmaceutically acceptable salt thereof.

11. A pharmaceutical composition according to claim 1, wherein the composition comprises the compound of formula (I), or a pharmaceutically acceptable salt thereof, and two therapeutically active ingredients acting as CFTR modulators, wherein one of said CFTR modulators is a CFTR potentiator or a pharmaceutically acceptable salt thereof, and, the second CFTR modulator is a CFTR corrector or a pharmaceutically acceptable salt thereof.

12. A pharmaceutical composition according to claim 1, wherein said composition comprises the compound of formula (I), or a pharmaceutically acceptable salt thereof, and
ivacaftor and tezacaftor, or pharmaceutically acceptable salts thereof; or
ivacaftor and lumacaftor, or pharmaceutically acceptable salts thereof; or
navocaftor and galicaftor, or pharmaceutically acceptable salts thereof.

13-15. (canceled)

16. A pharmaceutical composition according to claim 8, wherein the one or more therapeutically active ingredients acting as CFTR modulator(s) is/are a type-I corrector selected from lumacaftor, tezacaftor, and galicaftor; and/or a type-II corrector which is Corrector4a; and/or a type-III corrector selected from elexacaftor and vanzacaftor; and/or a CFTR potentiator selected from ivacaftor, navocaftor, icenticaftor, and deutivacaftor; or a pharmaceutically acceptable salt thereof.

17. A pharmaceutical composition according to claim 8, wherein said composition comprises one therapeutically active ingredient acting as CFTR modulator, wherein said CFTR modulator is a CFTR potentiator; or a pharmaceutically acceptable salt thereof.

18. A pharmaceutical composition according to claim 8, wherein the composition comprises two therapeutically active ingredients acting as CFTR modulators, wherein one of said CFTR modulators is a CFTR potentiator or a pharmaceutically acceptable salt thereof, and, the second CFTR modulator is a CFTR corrector or a pharmaceutically acceptable salt thereof.

19. A pharmaceutical composition according to claim 8, wherein said composition comprises (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide;
or a pharmaceutically acceptable salt thereof;
and
ivacaftor and tezacaftor, or pharmaceutically acceptable salts thereof; or
ivacaftor and lumacaftor, or pharmaceutically acceptable salts thereof; or
navocaftor and galicaftor, or pharmaceutically acceptable salts thereof.

20. A method of treatment of cystic fibrosis in a subject in need thereof, said method comprising the administration of an effective amount of a pharmaceutical composition as defined in claim 1 to the subject.

21. A method of treatment of cystic fibrosis in a subject in need thereof, said method comprising the administration of an effective amount of a pharmaceutical composition as defined in claim 8 to the subject.

22. A method of treatment of cystic fibrosis in a subject in need thereof, said method comprising the administration of an effective amount of a compound of Formula (I): 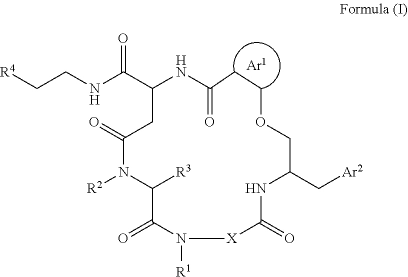
wherein
X represents —CR^{X1}R^{X2}, wherein R^{X1} represents hydrogen, and R^{X2} represents
C₁₋₆-alkyl;
C₁₋₄-fluoroalkyl; or
C₃₋₆-cycloalkyl;
R¹ represents C₁₋₄-alkyl;
R² represents C₁₋₄-alkyl;
R³ represents C₁₋₆-alkyl;
R⁴ represents 5-membered heteroaryl, wherein said 5-membered heteroaryl independently is unsubstituted, mono-, or di-substituted, wherein the substitutents are independently selected from C₁₋₄-alkyl, C₁₋₄-alkoxy, C₁₋₃-fluoroalkyl, C₁₋₃-fluoroalkoxy, C₃₋₆-cycloalkyl, or halogen;
Ar¹ represents 8- to 10-membered bicyclic heteroarylene, wherein said bicyclic heteroarylene independently is unsubstituted or mono-substituted with C₁₋₄-alkyl, or halogen;
Ar² represents phenyl, wherein said phenyl is unsubstituted, mono- or di-substituted wherein the substituents are independently selected from C₁₋₄-alkyl, C₁₋₃-fluoroalkyl, halogen, C₁₋₆-alkoxy, and C₁₋₃-fluoroalkoxy;
or a pharmaceutically acceptable salt thereof;
and an effective amount of one or more therapeutically active ingredients acting as CFTR modulator(s);
wherein said CFTR modulator(s) is/are one or more CFTR corrector(s), and/or a CFTR potentiator; or a pharmaceutically acceptable salt thereof.

23. A method of treatment of cystic fibrosis in a subject in need thereof, said method comprising the administration of an effective amount of (3S,7S,10R,13R)-13-benzyl-20-fluoro-7-isobutyl-N-(2-(3-methoxy-1,2,4-oxadiazol-5-yl)ethyl)-6,9-dimethyl-1,5,8,11-tetraoxo-10-(2,2,2-trifluoroethyl)-1,2,3,4,5,6,7,8,9,10,11,12,13,14-tetradecahydro-[1]oxa[4,7,10,14]tetraazacycloheptadecino[16,17-f]quinoline-3-carboxamide;
or a pharmaceutically acceptable salt thereof;
and an effective amount of one or more therapeutically active ingredients acting as CFTR modulator(s);
wherein said CFTR modulator(s) is/are one or more CFTR corrector(s), and/or a CFTR potentiator; or a pharmaceutically acceptable salt thereof.

24. A method according to claim 22, wherein said CFTR modulators are
ivacaftor and tezacaftor, or pharmaceutically acceptable salts thereof; or
ivacaftor and lumacaftor, or pharmaceutically acceptable salts thereof; or
navocaftor and galicaftor, or pharmaceutically acceptable salts thereof.

25. A method according to claim 23, wherein said CFTR modulators are
ivacaftor and tezacaftor, or pharmaceutically acceptable salts thereof; or
ivacaftor and lumacaftor, or pharmaceutically acceptable salts thereof; or
navocaftor and galicaftor, or pharmaceutically acceptable salts thereof.
